(19) United States
(12) Patent Application Publication
(10) Pub. No.: US 2023/0416816 A1
(43) Pub. Date: Dec. 28, 2023

(54) METHODS AND DEVICES FOR MULITPLEXED PROTEOMIC AND GENETIC ANALYSIS AND ON-DEVICE PREPARATION OF CDNA

(71) Applicant: IsoPlexis Corporation, Branford, CT (US)

(72) Inventors: Rui ZHENG, Madison, CT (US); Dana ASHWORTH, Branford, CT (US); Sean MACKAY, New Haven, CT (US)

(21) Appl. No.: 18/035,037

(22) PCT Filed: Nov. 3, 2021
(86) PCT No.: PCT/US2021/057902

§ 371 Date: May 2, 2023

Related U.S. Application Data
(60) Provisional application No. 63/241,791, filed on Sep. 8, 2021.
(60) Provisional application No. 63/147,017, filed on Feb. 8, 2021.
(60) Provisional application No. 63/112,432, filed on Nov. 11, 2020.
(60) Provisional application No. 63/109,218, filed on Nov. 3, 2020.

(51) Int. Cl. C12Q 1/6874; B01L 3/00; C12Q 1/6886; G01N 33/68; G01N 33/543

(52) U.S. Cl. CPC C12Q 1/6874; B01L 3/502761; C12Q 1/6886; G01N 33/6842; G01N 33/54313; C12Q 2600/16; C12Q 2600/158; G01N 2570/00; B01L 2200/0668

(57) ABSTRACT

Disclosed are devices and methods capable of multiplexed analysis of multiple cellular activities and pathways in single cells including genomic, transcriptomic, and proteomic analysis.

### CROSS-REFERENCE TO RELATED APPLICATIONS

This application claims the benefit of U.S. Application No. 63/109,218, filed on Nov. 3, 2020, U.S. Application No. 63/112,432, filed on Nov. 11, 2020, U.S. Application No. 63/147,017, filed on Feb. 8, 2021, and U.S. Application No. 63/241,791, filed on Sep. 8, 2021, the contents of which are herein incorporated by reference in their entirety.

### FIELD OF THE DISCLOSURE

The disclosure is directed to molecular biology, and more specifically to multiplexed methods of identifying and analyzing proteomic, transcriptomic, and genomic information in single cells.

### BACKGROUND

There have been long felt but unmet needs in the art for devices and methods capable of multiplexed analysis of single cells including the ability to simultaneously analyze proteomic, transcriptomic, and genomic information from a single cell. This disclosure provides devices and methods capable of analyzing multiple cellular activities and pathways in single cells in a high-throughput format wherein hundreds of intracellular components, including DNA, RNA, and protein levels, of each single cell of a plurality of thousands of cells are analyzed in parallel. The disclosure provides devices and methods to solve these long-felt but unmet needs.

### BRIEF SUMMARY

In some embodiments of this disclosure, a single consolidated automation system for multi-omics (e.g., proteomics, metabolomics, genomics, and transcriptomics) is provided. Such embodiments can be configured for (a) user choice from a variety of chip families to measure one or more “omes” (e.g., proteome, metabolome, genome, and transcriptome) from single cells and (b) end-to-end automation, including a plurality of, and in some embodiments, all of: cell loading, incubation, assay handling, and imaging, within a single system.

The present disclosure provides a multiplex assay chip device configured for multiplexed analysis of biological material, the device comprising: a plurality of capture beads (CB) each bead including a capture moiety; and a substrate having a plurality of chambers each including an open end arranged on a first side of the substrate, and a length, a width, and a depth (“dimensions”), a pocket P1 having a pocket diameter greater than the width of the chamber, and at least one CB arranged within the pocket of each chamber, wherein each CB includes a CB diameter smaller than the pocket diameter and larger than the width of the chamber; and a surface configured to couple with the first side of the substrate to cover each chamber.

The present disclosure provides a multiplex assay chip device configured for multiplexed analysis of biological material, the device comprising: a plurality of capture beads (CB) each bead including a capture moiety; a substrate having a plurality of chambers each including an open end arranged on a first side of the substrate, and a length, a width, and a depth (“dimensions”), a pocket P1 having a pocket diameter greater than the width of the chamber, and at least one CB arranged within the pocket of each chamber, wherein each CB includes a CB diameter smaller than the pocket diameter and larger than the width of the chamber; and a surface configured to couple with the first side of the substrate to cover each chamber, wherein the surface comprises a plurality of substantially parallel lines of capture antibodies, each line of capture antibodies comprises a specific and different antibody, relative to antibodies of adjacent lines of capture antibodies, configured to bind to a different target molecule, and at least one portion of each of the plurality of lines are arranged as to be exposed to each chamber.

The present disclosure provides a multiplex assay chip device configured for multiplexed analysis of biological material, the device comprising: a plurality of capture beads (CB) each bead including a capture moiety; a substrate having a plurality of chambers each including an open end arranged on a first side of the substrate, and a length, a width, and a depth (“dimensions”), a plurality of pockets comprising at least a first pocket P1 and a second pocket P2, each pocket of the plurality of pockets having a pocket diameter greater than the width of the chamber, and at least one CB arranged within each pocket of each chamber, wherein each CB includes a CB diameter smaller than the pocket diameter and larger than the width of the chamber; and a surface configured to couple with the first side of the substrate to cover each chamber.

The present disclosure provides a multiplex assay chip device configured for multiplexed analysis of biological material, the device comprising: a plurality of capture beads (CB) each bead including a capture moiety; a substrate having a plurality of chambers each including an open end arranged on a first side of the substrate, and a length, a width, and a depth (“dimensions”), a plurality of pockets comprising at least a first pocket P1 and a second pocket P2, each pocket of the plurality of pockets having a pocket diameter greater than the width of the chamber, and at least one CB arranged within each pocket of each chamber, wherein each CB includes a CB diameter smaller than the pocket diameter and larger than the width of the chamber; and a surface configured to couple with the first side of the substrate to cover each chamber, wherein the surface comprises a plurality of substantially parallel lines of capture antibodies, each line of capture antibodies comprises a different antibody, relative to antibodies of adjacent lines of capture antibodies, configured to bind to a different target molecule, and at least one portion of each of the plurality of lines are arranged as to be exposed to each chamber.

In some aspects, P1 and P2 have the same pocket diameter.

In some aspects, P1 and P2 have different pocket diameters.

In some aspects, each chamber has a width of between 1 μm and 100 μm. In some aspects, the chamber has a length of between 1 μm and 2000 μm.

In some aspects, the chamber has a depth of between 1 μm and 100 μm.

In some aspects, P1 has a pocket diameter of between 5 μm and 50 μm larger than the chamber width. In some aspects, P1 has a pocket diameter of between 10 μm and 100 μm. In some aspects, P2 has a pocket diameter of between 5 μm and 50 μm larger than the P1 pocket diameter.

In some aspects, P2 has a pocket diameter of between 15 μm and 150 μm. In some aspects, the diameter of the CB of P1, CB1, is between 0 μm and 50 μm smaller than the diameter of P1. In some aspects, the diameter of the CB of P2, CB2, is between 0 μm and 50 μm smaller than the diameter of P2.

In some aspects, the diameter of the CB in P2, CB2 is larger than the diameter of P1. In some aspects, the CB of P2, CB2, does not fit within P1. In some aspects, P1 and P2 are center-aligned with respect to the width of the chamber. In some aspects, P1 and P2 are not center-aligned with respect to the width of the chamber. In some aspects, P1 and P2 are non-overlapping. In some aspects, P1 is positioned at any point along the length of the chamber. In some aspects, P1 and P2 are positioned at any point along the length of the chamber.

In some aspects, P1 and P2 are any shape. In some aspects, P1 and P2 are circular, ovoid, rectangular, square, triangular, pentagonal, hexagonal, or octagonal. In some aspects, the plurality of pockets includes a third pocket P3 and a third CB, CB3.

In some aspects, P3 comprises a pocket diameter of between 5 and 50 μm larger than the P2 pocket diameter. In some aspects, P3 comprises a pocket diameter of between 20 and 200 μm. In some aspects, CB3 comprises a diameter of between 0 and 50 μm smaller than the P3 pocket diameter.

In some aspects, the capture moiety is configured to capture nucleic acid sequences, peptides, proteins, metabolites, or organic molecules. In some aspects, the capture moiety is configured to capture nucleic acid sequences.

In some aspects, the captured nucleic acid sequence is DNA, RNA, or a combination thereof. In some aspects, the DNA is autosomal DNA, chromosomal DNA, cDNA, exosome DNA, single stranded DNA, or double stranded DNA. In some aspects, the RNA is mRNA, rRNA, tRNA, snRNA, regulatory RNA, microRNA, exosome RNA, or double stranded RNA. In some aspects, the RNA is an mRNA. In some aspects, the RNA is a guide RNA from a CRISPR-Cas system.

In some aspects, the nucleic acid capturing CB is an oligonucleotide capture bead comprising a nucleic acid capture sequence tethered to a bead.

In some aspects, the nucleic acid capturing CB comprises from one to 10,000,000 capture nucleic acid sequences.

In some aspects, the nucleic acid capturing CB capture nucleic acid sequence comprises an individually unique cell barcode sequence, a PCR handle, a unique molecular identifier (UMI), a barcode handle sequence, and a capture sequence.

In some aspects, the capture sequence comprises a polyT sequence for mRNA polyA capture. In some aspects, the capture sequence comprises an rGrGrG capture sequence for mRNA capture.

In some aspects, the capture sequence comprises a gene-specific or sequence-specific capture sequence.

In some aspects, each nucleic acid sequence of the nucleic acid capturing CB comprises a unique UMI.

In some aspects, the cell barcode sequence of the CB is unique to each CB of the plurality of CBs. In some aspects, the cell barcode sequence of the CB is unique to each chamber of the plurality of chambers.

In some aspects, the CB is a protein capturing CB configured to capture proteins. In some aspects, the protein capturing CB comprises a capture antibody tethered to a bead.

In some aspects, the CB bead comprises plastic, polymer, metal, or silica. In some aspects, the CB bead comprises polystyrene. In some aspects, the bead is porous. In some aspects, the porous bead is configured to release one or more agents. In some aspects, the one or more agents can comprise an enzyme, catalyst, stimulatory agent, or therapeutic agent.

In some aspects, the surface comprises glass. In some aspects, the plurality of substantially parallel lines of capture antibodies comprises between 2 and 200 substantially parallel lines of capture antibodies. In some aspects, the plurality of substantially parallel lines of capture antibodies comprises between 2 and 50 substantially parallel lines of capture antibodies. In some aspects, the plurality of substantially parallel lines of capture antibodies comprises between 2 and 25 substantially parallel lines of capture antibodies.

In some aspects, the plurality of chambers comprises between 2 and 100,000 chambers. In some aspects, the plurality of chambers comprises between 1,000 and 100,000 chambers In some aspects, the plurality of chambers comprises between 1,000 and 12,000 chambers. In some aspects, the plurality of chambers comprises between 1,000 and 10,000 chambers. In some aspects, the plurality of chambers comprises between 2 and 5,000 chambers. In some aspects, the plurality of chambers comprises between 1,000 and 20,000 chambers

In some aspects, the substrate comprises a polymer. In some aspects, the polymer comprises polydimethylsiloxane (PDMS).

In some aspects, the biological material is a biological sample, a metabolite, a protein, a polypeptide, or a cell. In some aspects, the cell is a single cell. In some aspects, the single cell is a healthy cell, tumor cell, malignant cell, neural cell, glial cell, immune cell, T-cell, B-cell, bacterium, Chinese hamster ovary (CHO) cell, yeast cell, algal cell. In some aspects, the single cell is genetically-modified.

In some aspects, the biological sample is obtained from a subject. In some aspects, the biological sample comprises blood, cerebral spinal fluid (CSF), lymph fluid, plural effusion, urine or saliva. In some aspects, the biological sample comprises a cell culture media. In some aspects, the biological sample comprises a tissue sample or a tissue biopsy.

In some aspects, the subject is healthy, has cancer, has an autoimmune disorder, has an inflammatory disorder, has a neurological disorder, has a metabolic disorder, has a degenerative disorder, has a genetic mutation or epigenetic modification associated with a disease or disorder.

The present disclosure provides a multiplexed method for the simultaneous identification of: (a) at least one expressed gene or at least one T-cell receptor (TCR) sequence and (b) at least one cytokine from a single subject T-cell within a heterogeneous T-cell population comprising: (I) providing a device, wherein the device comprises: a substrate having a plurality of chambers each comprising at least one capture bead (CB); and a surface configured to couple with a first side of the substrate to cover each chamber, wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber; (II) detecting the at least one cytokine in a subject T-cell comprising the steps of: (a) contacting the subject T-cell and a stimulatory agent under conditions to permit stimulation of the subject T-cell; (b) introducing the subject T-cell to at least one chamber of the plurality of chambers, wherein the chamber is in fluid communication with the surface; (c) maintaining the subject T-cell in the chamber under conditions sufficient to permit: (1) the subject T-cell to secrete at least one cytokine and (2) at least one antibody of the antibody panel specific for the at least one protein to bind the at least one cytokine forming at least one antibody:cytokine complex; and (d) imaging the surface comprising the at least one antibody:cytokine complex, thereby identifying the one or more cytokine of the subject T-cell; (III) calculating a polyfunctional strength index (PSI) for the subject T-cell; (IV) selecting at least one subject T-cell with a PSI above a cutoff value; (V) identifying the at least one expressed gene or at least one TCR sequence of a subject T-cell comprising the steps of: (a) lysing the subject T-cell such that the subject T-cell releases at least one target RNA encoding for the at least one expressed gene or at least one TCR sequence under conditions sufficient for the target RNA to contact the CB to form an CB-target RNA complex, (b) determining the sequence of the complexed target RNA to determine the sequence of at least one expressed gene or at least one TCR sequence.

In some aspects, the method further comprises the step of disrupting contact between the subject T-cell and the target cell or the stimulatory agent.

In some aspects, the subject T-cell and the stimulatory agent are in fluid communication. In some aspects, the method further comprises comprising the step of removing the stimulatory agent.

In some aspects, the T-cell is a naïve T-cell, an activated T-cell, an effector T-cell, a helper T-cell, a cytotoxic T-cell, a gamma-delta T-cell, a regulatory T-cell, a memory T-cell, a memory stem T-cell, an engineered T-cell, a gene modified T-cell, a CAR-T-cell, or a TCR-T-cell.

In some aspects, the stimulatory agent comprises a stimulatory antibody. In some aspects, the stimulatory antibody is a monoclonal antibody. In some aspects, the monoclonal antibody is a fully human antibody. In some aspects, the monoclonal antibody is a humanized antibody, a chimeric antibody, a recombinant antibody or a modified antibody.

In some aspects, the modified antibody comprises one or more sequence variations when compared to a fully human version of an antibody having the same epitope specificity, one or more modified or synthetic amino acids, or a chemical moiety to enhance a stimulatory function.

In some aspects, the stimulatory antibody specifically binds an epitope of a T-cell regulator protein.

In some aspects, the T-cell regulator protein comprises programmed cell death protein 1 (PD-1). In some aspects, the stimulatory antibody comprises Nivolumab or a biosimilar thereof. In some aspects, the stimulatory agent comprises a stimulatory ligand. In some aspects, the stimulatory ligand comprises programmed death ligand 1 (PD-L1).

In some aspects, the conditions are sufficient to permit (1) the subject T-cell to secrete at least one cytokine and (2) at least one antibody of the antibody panel specific for the at least one protein to bind the at least one cytokine, forming at least one antibody:cytokine complex, and wherein the conditions comprise 5% CO₂ and 37° C. for a period of at least 2 hours. In some aspects, the period is at least 4 hours, at least 8 hours, at least 12 hours, at least 16 hours, or at least 24 hours.

In some aspects, the at least one chamber comprises a cell media that maintains the viability of the subject T-cell from step II(b) through II(d).

In some aspects, the PSI is the sum of all cytokine levels of a cell. In some aspects, a subject T-cell that secretes at least two cytokines is a polyfunctional subject T-cell. In some aspects, the at least two cytokines produced by the polyfunctional subject T-cell comprise the same cytokines. In some aspects, an increased PSI indicates an increase in the potency or functionality of the polyfunctional subject T-cells.

In some aspects, the one or more cytokines are selected from the group consisting of effector, stimulatory, regulatory, inflammatory, or chemoattractive cytokines. In some aspects, at least one cytokine is an effector cytokine selected from the group consisting of Granzyme B, IFN-γ, M1P-1α, Performin, TNF-α, and TNF-β. In some aspects, at least one cytokine is a stimulatory cytokine are selected from the group consisting of GM-CSF, IL-12, IL-15, IL-2, IL-21, IL-5, IL-7, IL-8 and IL-9. In some aspects, at least one cytokine is a chemoattractive cytokine selected from the group consisting of CCL-11, IP-10, MIP-1β and RANTES. In some aspects, at least one cytokine is a regulatory cytokine selected from the group consisting of IL-10, IL-13, IL-22, IL-4, TGF-β1, sCD137 and sCD40L. In some aspects, at least one cytokine is an inflammatory cytokine selected from the group consisting of IL-17A, IL-17F, IL-1β, IL-6, MCP-1 and MCP-4.

The disclosure provides method of creating a T-cell therapeutic comprising the at least one expressed gene or TCR sequence of methods of the disclosure, comprising: (I) transducing the at least one expressed gene or TCR sequence into a T-cell; (II) expanding the T-cell.

The present disclosure provides a multiplexed method for the simultaneous identification of at least one expressed gene or at least one B-cell receptor (BCR) sequence and at least one cytokines from a single subject B-cell within a heterogeneous B-cell population comprising: (I) providing a device comprising: a substrate having a plurality of chambers each comprising at least one capture bead (CB); and a surface configured to couple with a first side of the substrate to cover each chamber, wherein: the surface comprising a plurality of substantially parallel lines of capture antibodies, each line of capture antibodies comprising a specific and different antibody, configured to bind to a different target molecule, and at least one portion of each of the plurality of substantially parallel lines are arranged as to be exposed to each chamber; (II) detecting the at least one cytokine in a subject B-cell comprising the steps of: (a) contacting the subject B-cell and a stimulatory agent under conditions to permit stimulation of the subject B-cell; (b) introducing the subject B-cell to at least one chamber of the plurality of chambers, wherein the chamber is in fluid communication with the surface; (c) maintaining the subject B-cell in the chamber under conditions sufficient to permit: (1) the subject B-cell to secrete at least one cytokine and (2) at least one antibody of the antibody panel specific for the at least one protein to bind the at least one cytokine forming at least one antibody:cytokine complex; and (e) imaging the surface comprising the at least one antibody:cytokine complex, thereby identifying the one or more cytokine of the subject B-cell; (III) calculating a polyfunctional strength index (PSI) for the subject B-cell; (IV) selecting at least one subject B-cell with a PSI above a pre-determined cutoff value; (V) identifying the at least one BCR sequence of a subject B-cell comprising the steps of: (a) lysing the subject B-cell such that the subject B-cell releases at least one target RNA encoding for the at least one BCR sequence under conditions sufficient for the target RNA to contact the CB to form an CB-target RNA complex, (b) determining the sequence of the complexed target RNA to determine the sequence of the component of the at least one BCR sequence.

In some aspects, the method further comprises the step of disrupting contact between the subject B-cell and the target cell or the stimulatory agent. In some aspects, the subject B-cell and the target cell are comprised by a composition and wherein the subject B-cell and the target cell or the stimulatory agent are in fluid communication.

In some aspects, the method further comprises the step of depleting the target cell or the stimulatory agent from the composition.

In some aspects, the B-cell is a hybridoma, a genetically modified B-cell, a transitional B-cell, a naïve B-cell, a plasma B-cell, a B-1 cell, a B-2 cell, a regulatory B-cell, or a memory B-cell. In some aspects, the stimulatory agent comprises a stimulatory antibody.

In some aspects, the stimulatory antibody specifically binds an epitope of a T cell regulator protein. In some aspects, the T cell regulator protein comprises programmed cell death protein 1 (PD-1). In some aspects, the stimulatory antibody comprises Nivolumab or a biosimilar thereof. In some aspects, the stimulatory agent comprises a stimulatory ligand. In some aspects, the stimulatory ligand comprises programmed death ligand 1 (PD-L1).

In some aspects, the conditions sufficient to permit (1) the subject B-cell to secrete at least one cytokine and (2) at least one antibody of the antibody panel specific for the at least one protein to bind the at least one peptide, polypeptide, or protein, forming at least one antibody:cytokine complex, and wherein the conditions comprise 5% CO₂ and 37° C. for a period of at least 2 hours. In some aspects, the period is at least 4 hours, at least 8 hours, at least 12 hours, at least 16 hours, or at least 24 hours.

In some aspects, the at least one chamber comprises a cell media that maintains the viability of the subject B-cell from step II(b) through II(d).

In some aspects, the PSI is the sum of all cytokine levels of a cell. In some aspects, a subject B-cell that secretes at least two cytokines is a polyfunctional subject B-cell. In some aspects, the at least two cytokines produced by the polyfunctional subject B-cell comprise the same cytokines. In some aspects, an increased PSI indicates an increase in the potency of the polyfunctional subject B-cells. In some aspects, the one or more cytokines are selected from the group consisting of effector, stimulatory, regulatory, inflammatory, or chemoattractive cytokines. In some aspects, at least one cytokine is an effector cytokine selected from the group consisting of Granzyme B, IFN-γ, M1P-la, Performin, TNF-α, and TNF-β.

In some aspects, at least one cytokine is a stimulatory cytokines are selected from the group consisting of GM-CSF, IL-12, IL-15, IL-2, IL-21, IL-5, IL-7, IL-8 and IL-9. In some aspects, at least one cytokine is a chemoattractive cytokine selected from the group consisting of CCL-11, IP-10, MIP-1β and RANTES. In some aspects, at least one cytokine is a regulatory cytokine selected from the group consisting of IL-10, IL-13, IL-22, IL-4, TGF-β1, sCD137 and sCD40L. In some aspects, at least one cytokine is an inflammatory cytokine selected from the group consisting of IL-17A, IL-17F, IL-1β, IL-6, MCP-1 and MCP-4.

The present disclosure provides a method of creating a B-cell therapeutic comprising the at least one expressed gene or BCR sequence identified via methods of the disclosure comprising: (I) transducing the at least one expressed gene or BCR sequence into a B-cell; (II) expanding the B-cell.

The present disclosure provides a multiplexed method for determining a resistance pathway in a cancerous cell that confers resistance to a therapeutic agent comprising the identification of: (a) at least one nucleic acid sequence and (b) at least one protein from a single cancer cell resistant to at least one therapeutic agent, comprising: (I) providing a device comprising: a substrate having a plurality of chambers each comprising at least one capture bead (CB); and a surface configured to couple with a first side of the substrate to cover each chamber wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber; (II) introducing the single cancer cell to a chamber of the plurality of chambers, wherein the chamber is in fluid communication with the surface; (III) maintaining the cancer cell in the chamber under conditions sufficient to cause cell lysis to produce a cell lysate; (IV) detecting the at least one protein in the cancer cell comprising the steps of: (a) incubating the cell lysate in the chamber under conditions sufficient to allow at least one antibody and at least one protein to form an antibody:protein complex; and (b) imaging the surface comprising the at least one antibody:protein complex, thereby identifying the one or more protein of the cancer cell; (V) identifying the at least one nucleic acid sequence of the cancer cell comprising the steps of: (a) incubating the cell lysate in the chamber under conditions sufficient for the at least one nucleic acid sequence to contact the CB to form an CB-nucleic acid sequence complex, (b) determining the sequence of the complexed nucleic acid sequence; (VI) performing a computational correlation of the at least one protein and at least one nucleic acid sequence such that a pathway that confers resistance to the therapeutic agent in the cancer cell is identified.

In some aspects, the cancer cell is a cultured cancer cell, a primary tumor sample, a circulating tumor cell, a blood cancer. In some aspects, the cancer cell is a carcinoma, sarcoma, leukemia, lymphoma, myeloma, or melanoma. In some aspects, resistance to a therapeutic agent in the cancerous cell is measured by a reduction in efficacy of the therapeutic agent.

In some aspects, resistance to a therapeutic agent in the cancerous cell is measured by a reduction in potency of the therapeutic agent.

In some aspects, resistance to a therapeutic agent in the cancerous cell results in a cancerous cell that is non-responsive to treatment with the therapeutic agent. In some aspects, resistance to a therapeutic agent in the cancerous cell results in reduced cell death of a cancerous cell treated with the therapeutic agent. In some aspects, resistance to a therapeutic agent in the cancerous cell results in a patient that is non-responsive to treatment with the therapeutic agent.

In some aspects, the resistance to the at least one therapeutic agent results from treatment of a mammalian patient with the therapeutic agent.

In some aspects, the resistance to the at least one therapeutic agent is induced in vitro. In some aspects, the in vitro resistance is induced by contacting the cancer cell with the at least one therapeutic agent under conditions sufficient to cause resistance. In some aspects, the in vitro resistance is induced via gene editing.

In some aspects, the gene editing is a sequence-specific gene editing method. In some aspects, the gene editing comprises contacting the cell with a composition comprising a CRISPR-Cas composition, zinc-finger nuclease, TALEN, PUF, PUMBY, or meganuclease.

In some aspects, the in vitro resistance is induced via gene silencing. In some aspects, the gene silencing comprises contacting the cell with CRISPR-Cas composition, RNAi composition, siRNA composition, ribozyme composition, or miRNA.

In some aspects, the capture antibody surface comprises antibodies specific for proteins comprising a cell signaling pathway. In some aspects, the cell signaling pathway is targeted by the at least one therapeutic agent.

In some aspects, the identifying at least one nucleic acid sequence comprises from about one to about 1,000,000 nucleic acid sequences.

In some aspects, the at least one nucleic acid sequence is an mRNA sequence.

In some aspects, the computational correlation comprises a dimensionality reduction technique. In some aspects, the dimensionality reduction technique comprises at least one of a principal component analysis (PCA), t-distributed stochastic neighbor embedding (TSME), or uniform manifold approximation and projection (UMAP).

In some aspects, computational correlation of the at least one protein and at least one nucleic acid sequence identifies compensatory pathways induced by resistance to the at least one therapeutic agent.

In some aspects, the CB comprises a capture nucleic acid sequence comprising an individually unique cell barcode sequence comprising a predetermined number of base pairs, a PCR handle, a unique molecular identifier (UMI), a barcode handle sequence, and a polyT sequence or capture sequence. In some aspects, the CB comprises from one to 10,000,000 capture nucleic acid sequences. In some aspects, the cell barcode sequence of the CB is unique to each CB of the plurality of CBs.

In some aspects, the cell barcode sequence of the CB is unique to each chamber of the plurality of chambers. In some aspects, each nucleic acid sequence of the CB comprises a unique UMI. In some aspects, the individually unique cell barcode of the CB is sequenced.

In some aspects, sequencing the individually unique cell barcode sequence comprises synthesizing a cDNA barcode sequence. In some aspects, synthesizing the cDNA barcode sequence comprises contacting the sequence encoding the barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some aspects, the sequence encoding the cell barcode comprises between 2 and 20 nucleotides. In some aspects, the sequence encoding the cell barcode comprises 12 nucleotides.

In some aspects, the conditions sufficient for hybridization and cDNA synthesis comprise a plurality of deoxynucleotides (dNTPs). In some aspects, at least one dNTP of the plurality of deoxynucleotides (dNTPs) comprises a modification. In some aspects, each dNTP of the plurality of deoxynucleotides (dNTPs) comprises a modification.

In some aspects, the modification comprises a label. In some aspects, the label comprises a fluorophore or a chromaphore. In some aspects, the label comprises a fluorophore. In some aspects, each adenine comprises a first label, wherein each cytosine comprises a second label, each guanine comprises a third label, and each thymine comprises a fourth label. In some aspects, the first label, the second label, the third label, and the fourth label are distinct labels. In some aspects, the first label, the second label, the third label, and the fourth label are spectrally-distinguishable fluorescent labels. In some aspects, the first label, the second label, the third label are spectrally-distinguishable fluorescent labels and the fourth nucleotide has no label.

In some aspects, the capture sequence comprises a polyT sequence for mRNA polyA capture. In some aspects, the capture sequence comprises an rGrGrG capture sequence for mRNA capture. In some aspects, the capture sequence comprises a gene-specific capture sequence.

In some aspects, the nucleic acid encoding the barcode further comprises a sequence encoding a template switch oligonucleotide (TSO) hybridization site. In some aspects, the sequence encoding a TSO hybridization site comprises a poly-riboguanine (poly-rG) sequence.

In some aspects, the method further comprises contacting the nucleic acid sequence encoding the barcode of the CB and a TSO under conditions sufficient for hybridization of the TSO to a portion of the nucleic acid encoding the barcode to produce a nucleic acid/TSO duplex. In some aspects, the TSO comprises a sequence complementary to the sequence encoding the UMI, a sequence complementary to the sequence encoding the TSO handle, a sequence complementary to the sequence encoding the sequence encoding a TSO hybridization site, and a sequence complementary to a nucleic acid sequence of the cancer cell.

In some aspects, sequencing comprises synthesizing cDNA sequences comprising the complexed nucleic acid sequences, or a complement thereof, for each of the complexed nucleic acid sequences. In some aspects, sequencing further comprises removing the cDNA sequences from the chamber. In some aspects, sequencing further comprises amplifying the cDNA sequences by PCR.

In some aspects, the sequencing method is next generation sequencing (NGS). In some aspects, the method further comprises analyzing the cDNA sequences. In some aspects, the cDNA sequences are clustered by cell barcode sequence. In some aspects, the cDNA sequences are quantified by a bioanalyzer.

The disclosure provides a method of treating drug resistance in a cancer cell, comprising contacting the cell with at least one therapeutic agent targeting at least one pathway or protein belonging to a resistance pathway identified in methods of the disclosure.

The disclosure provides a multiplexed method for the simultaneous identification of: (a) at least one nucleic acid sequence, and (b) at least one protein from a single subject cell comprising: (I) providing a device comprising: a substrate having a plurality of chambers each comprising at least one capture bead (CB), wherein a first CB, CB1, is configured to capture a target nucleic acid; and a surface configured to couple with a first side of the substrate to cover each chamber wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber; (II) introducing the subject cell to a chamber of the plurality of chambers; (III) maintaining the subject cell in the chamber under conditions sufficient to cause cell lysis to produce a cell lysate; (IV) identifying at least one first nucleic acid sequence, comprising: (a) providing conditions sufficient for the target nucleic acid to contact the CB1 to form a CB1-target nucleic acid complex, (b) determining the sequence of the complexed target nucleic acid sequence to determine the sequence of the at least one expressed target nucleic acid sequence (V) detecting the at least one protein in the subject cell comprising the steps of: (a) incubating the cell lysate in the chamber under conditions sufficient to allow at least one antibody and at least one protein to form an antibody:protein complex; and (b) imaging the surface comprising the at least one antibody:protein complex, thereby identifying the one or more protein of the subject cell.

The disclosure provides a multiplexed method for the simultaneous identification of: (a) at least one nucleic acid sequence, and (b) at least one protein from a single subject cell comprising: (I) providing a device comprising: a substrate having a plurality of chambers each comprising at least two capture beads (CB), wherein a first CB, CB1, and a second CB, CB2, each are configured to capture different cellular components; and a surface configured to couple with a first side of the substrate to cover each chamber; (II) introducing the subject cell to a chamber of the plurality of chambers; (III) maintaining the subject cell in the chamber under conditions sufficient to cause cell lysis to produce a cell lysate; (IV) identifying at least one first nucleic acid sequence, comprising: (a) providing conditions sufficient for the target nucleic acid to contact the CB1 to form a CB1-target nucleic acid complex, (b) determining the sequence of the complexed target nucleic acid sequence to determine the sequence of the at least one expressed target nucleic acid sequence (V) detecting at least one second target protein or nucleic acid sequence comprising: (a) providing conditions sufficient for the protein or nucleic acid sequence to contact the CB2 to form a CB2-target protein or nucleic acid complex, (b) detecting the complexed target protein or nucleic acid sequence.

The disclosure provides a multiplexed method for the simultaneous identification of: (a) at least one nucleic acid sequence, and (b) at least one protein from a single subject cell comprising: (I) providing a device comprising: a substrate having a plurality of chambers each comprising at least two capture beads (CB), wherein a first CB, CB1, and a second CB, CB2, each are configured to capture different cellular components; and a surface configured to couple with a first side of the substrate to cover each chamber, wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber; (II) introducing the subject cell to a chamber of the plurality of chambers; (III) maintaining the subject cell in the chamber under conditions sufficient to cause cell lysis to produce a cell lysate; (IV) detecting the at least one protein in the subject cell comprising the steps of: (a) incubating the cell lysate in the chamber under conditions sufficient to allow at least one antibody and at least one protein to form an antibody:protein complex; and (b) imaging the surface comprising the at least one antibody:protein complex, thereby identifying the one or more protein of the subject cell; (V) identifying at least one first target nucleic acid sequence, comprising: (a) providing conditions sufficient for the target nucleic acid sequence to contact the CB1 to form a CB1-target nucleic acid complex, and (b) determining the sequence of the complexed target nucleic acid sequence to determine the sequence of the at least one expressed target nucleic acid sequence; (VI) detecting at least one second target protein or nucleic acid sequence comprising: (a) providing conditions sufficient for the protein or nucleic acid sequence to contact the CB2 to form a CB2-target protein or nucleic acid complex, and (b) detecting the complexed protein or nucleic acid sequence.

In some aspects, the CB2 is configured to capture a protein. In some aspects, the CB2 is configured to capture a nucleic acid.

In some aspects, detecting the nucleic acid further comprises sequencing the nucleic acid sequence.

In some aspects, the detecting the protein comprises contacting the CB2-target protein complex with a labeled secondary antibody and imaging the labeled secondary antibody. In some aspects, the first target nucleic acid sequence is DNA or RNA. In some aspects, the second target nucleic acid sequence is DNA or RNA.

In some aspects, the DNA is accessible genomic DNA, autosomal DNA, chromosomal DNA, cDNA, exosome DNA, single stranded DNA, or double stranded DNA. In some aspects, the RNA is mRNA, rRNA, tRNA, snRNA, regulatory RNA, microRNA, exosome RNA, or double stranded RNA. In some aspects, the RNA is an mRNA. In some aspects, the RNA is a guide RNA from a CRISPR-Cas system.

In some aspects, the first and second target nucleic acid sequences are not identical.

The disclosure provides a multiplexed method for the simultaneous identification of: (a) at least one nucleic acid sequence, and (b) at least one protein from a single subject B-cell comprising: (I) providing a device comprising: a substrate having a plurality of chambers each comprising at least two capture beads (CB), wherein a first CB is configured to capture proteins and a second CB is configured to capture nucleic acids, and a surface configured to couple with a first side of the substrate to cover each chamber; (II) introducing the subject B-cell to a chamber of the plurality of chambers; (III) maintaining the subject B-cell in the chamber under conditions sufficient to cause cell lysis to produce a cell lysate; (IV) identifying the at least one nucleic acid sequence comprising: (a) providing conditions sufficient for the target RNA to contact the first CB to form a first CB-target RNA complex, and (b) determining the sequence of the complexed target RNA sequence to determine the sequence of the at least one expressed target RNA sequence; and (V) detecting the at least one protein in the subject B-cell comprising the steps of: (a) incubating the cell lysate in the chamber under conditions sufficient to allow at least one protein to contact the first CB to form a first CB:protein complex, and (b) imaging the substrate comprising the at least one first CB:protein complex, thereby identifying the at least protein of the subject B-cell.

In some aspects, the second CB is configured to capture a protein. In some aspects, the second CB is configured to capture an antibody secreted by the subject B-cell. In some aspects, the antibody capture bead comprises a capture moiety comprising a polypeptide sequence encoding an antigen or epitope. In some aspects, the antibody secreted by the subject B-cell binds the antigen or epitope capture moiety. In some aspects, the antibody capture bead comprises a capture moiety comprises an antibody that binds a portion of an antibody constant region.

In some aspects, the antibody specific for an antibody constant region binds a portion of a heavy chain constant region or a light chain constant region. In some aspects, the second CB is configured to capture a nucleic acid. In some aspects, the method further comprises detecting at least one additional protein, wherein the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber; and detecting the at least one additional protein in the subject cell comprising the steps of: (a) incubating the cell lysate in the chamber under conditions sufficient to allow at least one antibody and at the least one additional protein to form an antibody:protein complex, (and (b) imaging the surface comprising the at least one antibody:protein complex, thereby identifying the at least one additional protein.

In some aspects, an individually unique cell barcode sequence of the CB is sequenced. In some aspects, sequencing the individually unique cell barcode sequence comprises synthesizing a cDNA barcode sequence.

In some aspects, synthesizing the cDNA barcode sequence comprises contacting the sequence encoding a barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some aspects, the sequence encoding the cell barcode comprises between 2 and 20 nucleotides. In some aspects, the sequence encoding the cell barcode comprises 12 nucleotides.

In some aspects, the conditions sufficient for hybridization and cDNA synthesis comprise a plurality of deoxynucleotides (dNTPs). In some aspects, at least one dNTP of the plurality of deoxynucleotides (dNTPs) comprises a modification. In some aspects, each dNTP of the plurality of deoxynucleotides (dNTPs) comprises a modification.

In some aspects, the modification comprises a label. In some aspects, the label comprises a fluorophore or a chromophore. In some aspects, the label is a fluorescent label. In some aspects, each adenine comprises a first label, wherein each cytosine comprises a second label, each guanine comprises a third label, and each thymine comprises a fourth label. In some aspects, at least one dNTP comprises a label, at least two dNTP comprise a label, at least three dNTP comprise a label, or at least 4 dNTP comprise a label. In some aspects, at least one dNTP remains unlabeled while the remaining dNTP comprise labels. In some aspects, the dNTP are selected from adenine, cytosine, guanine, thymine and/or uracil. In some aspects, the first label, the second label, the third label, and the fourth label are distinct labels. In some aspects, the first label, the second label, the third label, and the fourth label are spectrally-distinguishable fluorescent labels.

In some aspects, the nucleic acid encoding the barcode further comprises a sequence encoding a TSO hybridization site. In some aspects, the sequence encoding a TSO hybridization site comprises a poly-riboguanine (poly-rG) sequence. In some aspects, the methods of the disclosure further comprises contacting the nucleic acid sequence encoding the barcode of the CB and a TSO under conditions sufficient for hybridization of the TSO to a portion of the nucleic acid encoding the barcode to produce a nucleic acid/TSO duplex. In some aspects, the TSO comprises a sequence complementary to the sequence encoding the UMI, a sequence complementary to the sequence encoding the TSO handle, a sequence complementary to the sequence encoding the sequence encoding a TSO hybridization site, and a sequence complementary to the target nucleic acid sequences.

In some aspects, sequencing comprises synthesizing a cDNA sequence comprising one of the complexed target nucleic acid sequences, or a complement thereof, for each of the complexed target nucleic acid sequences.

In some aspects, the cDNA sequence comprises the target nucleic acid sequence, UMI, and individually unique cell barcode sequence. In some aspects, the cDNA sequence comprises the target nucleic acid sequence, or a complement thereof, UMI, and individually unique cell barcode sequence. In some aspects, sequencing comprises removing the cDNA sequences from the chamber. In some aspects, sequencing comprises amplifying the cDNA sequences by PCR.

In some aspects, the sequencing method is next generation sequencing (NGS). In some aspects, the method further comprises analyzing the cDNA sequences.

The disclosure provides a multiplex assay chip device configured for multiplexed analysis of biological material, comprising: a plurality of capture beads (CBs), each CB having an individually unique cell barcode sequence comprising a predetermined number of base pairs and a PCR handle, a unique molecular identifier, a barcode handle sequence, and a poly T sequence/capture sequence; a substrate having a plurality of chambers, each chamber comprising an open end arranged on a first side of the substrate, a length, a width, and a depth (“dimensions”), at least one pocket arranged therein having a pocket diameter greater than the width of the chamber, wherein the at least one pocket is alternately arranged and offset from at least one pocket of an adjacent chamber and at least one of the CBs arranged within the pocket of each chamber, wherein a diameter of each CB is smaller than the pocket diameter but larger than the width of the chamber, such that, each pocket and each CB are configured such that the CB is arranged approximately in the center of each chamber; and a surface configured to couple with the first side of the substrate to cover each chamber, the surface comprising a plurality of substantially parallel lines of capture antibodies, each line of capture antibodies comprising a specific and different antibody relative to antibodies of adjacent lines of capture antibodies, wherein the antibodies are configured to bind to a different target molecule and at least one portion of each of the plurality of lines are arranged as to be exposed to each chamber.

The disclosure provides a multiplex assay chip device manufacturing method, comprising: providing a plurality of capture beads (CBs), each CB having an individually unique cell barcode sequence comprising a predetermined number of base pairs and a PCR handle, a unique molecular identifier, a barcode handle sequence, and a poly T sequence/capture sequence; providing a chip substrate having a plurality of chambers, each chamber comprising an open end arranged on a first side of the substrate, a length, a width, and a depth (“dimensions”), at least one pocket arranged therein having a pocket diameter greater than the width of the chamber wherein the at least one pocket is alternately arranged and offset from at least one pocket of an adjacent chamber, at least one of the CBs, and preferably, a single CB, loaded within each chamber and preferably, within the pocket of each chamber, wherein a diameter of each CB is smaller than the pocket diameter but larger than the width of the chamber and each pocket and each CB are configured such that the CB is arranged approximately in the center of each chamber; coupling a chip surface to the first side of the substrate to cover each chamber, wherein the chip surface comprises a plurality of substantially parallel lines of capture antibodies, each line of capture antibodies comprising a specific and different antibody relative to antibodies of adjacent lines of capture antibodies, the capture antibodies being configured to bind to a different target molecule, at least one portion of each of the plurality of lines being arranged as to be exposed to each chamber, and the combination of the chip surface and chip substrate form an assay chip device; imaging the chip such that each unique cell barcode sequence of each OCB is read and to obtain data detailing the location of each CB and each pocket on the chip; and storing the data.

In some aspects, loading the CBs into the pockets comprises immobilizing the CBs therein.

In some aspects, loading the CBs into the pockets comprises providing the CBs within a first buffer solution; placing the chip device onto a rocker device; loading the buffer into a flow cell, such that one CB settles within each pocket via at least one of gravity and pocket diameter and relative CB diameter, wherein the rocker device is configured to encourage an CB to load into each pocket; washing excess beads from the chip device via a second buffer solution; drying the chip device at a first predetermined temperature; heating an agarose material to dissolve in TBE buffer; transferring the agarose material at a predetermined second temperature lower than the first predetermined temperature; spin-coating the chip device via centrifugation at increasing speeds for a first predetermined period of time; and cooling the agarose material at a third predetermined temperature, such that, the agarose material gels so as to immobilize each CB in each respective chamber, and gelling occurs within a predetermined time period.

The disclosure provides a capture-bead barcode sequence determination method for determining the barcode sequences on capture beads (CBs) immobilized on a chip device comprising: providing the device of any embodiment of the disclosure; obtaining the sequence of the cell barcode sequence of the oligonucleotide of each CB; and correlating the sequence of the cell barcode sequence of each CB to a spatial location of each respective the open rectangular chamber to produce a spatial map of the chip device.

The disclosure provides a device for loading capture beads (CBs) into chambers of an assay chip device of any preceding claim, comprising a flow cell, the flow cell comprising: a clamp comprising a first side and a second side; a housing corresponding to the second side to the clamp, and configured to retain the chip surface at a first side of the housing, and retain the chip substrate in a spaced apart arrangement from the chip surface, a void therebetween configured as a flow area; an inlet port arranged on a first end of the housing configured to receive a solution containing a plurality of biological cells, the inlet port being in fluid communication with a first end of the flow area; and an outlet port arranged on a second end of the housing and configured to exhaust the solution from the flow area, the outlet port being in fluid communication with a second end of the flow area, wherein upon the solution flowing through the flow area, whereby individual biological cells are distributed adjacent a plurality of the chambers, the clamp is configured such that the second side is advanced toward the first side, driving individual biological cells into respective chambers on the substrate, and, the chip surface is coupled to the chip substrate.

The disclosure provides a device for performing cell lysis to a plurality of individual cells arranged in a plurality of respective chambers of an assay chip device comprising: a clamp comprising a first side and a second side; a housing corresponding to the second side of the clamp, the clamp being configured to retain the chip surface at a first side of the housing and retain the chip substrate in a spaced apart arrangement from the chip surface, a void therebetween configured as a flow area, the flow area including a central portion corresponding to chip substrate and a perimeter portion surrounding a perimeter of the assay chip device; an inlet port arranged on a first end of the housing and configured to receive a lysis solution, the inlet port being in fluid communication with a first end of the flow area; and an outlet port arranged on a second end of the housing and configured to exhaust the lysis solution from the flow area, the outlet port being in fluid communication with a second end of the flow area, wherein when the clamp is in a first configuration, the chip surface is coupled to the chip substrate and the central portion of the flow area is inaccessible, the lysis solution flowing into the inlet port flows into the perimeter portion of the flow area and toward the outlet port, thereby filling the perimeter portion to a predetermined amount, and when the clamp is in a second configuration, the chip surface is decoupled from the chip substrate, the central portion of the flow area is accessible, and the lysis solution from the perimeter portion of the flow area diffuses into the central portion of the flow area, thereby allowing cells in each chamber to contact the lysis solution.

The disclosure provides a multiplexed proteomic and nucleic acid identification method comprising: providing the chip device of any embodiment of the disclosure; mapping chambers of the chip device via the process of claim 250 so as to form a map of the chip device and the location of each CB; storing the map; loading a single cell within each of the plurality of chambers of the chip device; lysing the single cell in each chamber such that the single cell releases a plurality of cellular components comprising: nucleic acid(s), and at least one of one or more cellular proteins and one or more target molecules of the plurality of cellular components; identifying at least one of the cellular proteins and target molecules by: providing reaction conditions configured to enable the formation of at least one antibody complex from respective interactions between: at least one of the cellular proteins and at least one capture antibody from the plurality of lines of capture antibodies, or at least one target molecule of the plurality of cellular components and the at least one capture antibody from the plurality of lines of capture antibodies, and providing at least one labeled secondary antibody that specifically binds to the at least one capture antibody complex to provide a detectable signal thereof, the detectable signal comprising a signature of the protein or target molecule forming the antibody complex; identifying the nucleic acid by: providing reaction conditions configured to enable nucleic acid to bind with the capture sequence of the CB forming one or more respective captured nucleic acids; and preparing cDNA of the captured cellular nucleic acids comprising the steps of: performing reverse transcription of the one or more respective captured cellular nucleic acids comprising hybridizing a reverse transcription primer to the PCR handle of the CB, wherein each cDNA comprises the cell barcode sequence of the CB; removing cDNA from individual CBs; amplifying cDNA to form a cDNA library by performing PCR; sequencing cDNA library; and spatially locating each cDNA via the map.

The disclosure provides a system, apparatus, device, or method according to any of the disclosed embodiments.

The disclosure provides a method for on-chip cDNA processing comprising: providing a flow-cell chip comprising: a plurality of capture beads (CBs), each configured to capture RNA; a substrate having a plurality of chambers, wherein each chamber includes at least one of the CBs arranged therein; and a surface configured to couple with the first side of the substrate to cover each chamber, wherein the surface optionally comprises a plurality of substantially parallel lines of capture antibodies, wherein: the surface is removably coupled to the substrate, such that the chip includes an open configuration, and a closed configuration; opening the chip corresponds to causing the chip to proceed from the closed configuration to the open configuration; closing the chip corresponds to causing the chip to proceed from the open configuration to the closed configuration, and in the open configuration, materials can flow into and/or out of the chambers; providing a single cell in a plurality of the chambers; while in the closed configuration, lysing the single cell within the plurality of chambers so as to release RNA; opening the chip and directing one or more reagents into the plurality of chambers configured for a reverse transcription reaction; closing the chip such that in the closed configuration, RNA reverse transcription occurs forming cDNA; removing the cDNA from each of the plurality of chambers; and opening the chip and removing the cDNA from the chip.

In some aspects, the CBs configured to capture RNA comprise a poly T sequence.

In some aspects, the one or more reagents for the reverse transcription reaction comprise a deoxyribonucleotide triphosphate mixture comprising adenine, guanine, cytosine, and thymine, a reverse transcriptase, and a reverse transcription primer.

In some aspects, the one or more reagents for the reverse transcription reaction further comprise Ficoll and one or more reverse transcription buffers. In some aspects, RNA reverse transcription comprises hybridizing the reverse transcription primer to a reverse transcription hybridization site on the CB. In some aspects, RNA reverse transcription comprises a thermal cycling step.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration of a device configured for simultaneous genomic, transcriptomic, and proteomic analysis of single cells according to compositions and methods of the disclosure.

In some embodiments, as shown in FIG. 2A, a method to manufacture barcoded micro-beads (in some embodiments, barcoded DNA micro-beads), for loading into assay chip-devices, and in some embodiments, into single-cell chambers of a substrate (see, e.g., steps 1-3). FIG. 2A also illustrates steps according to other embodiments which, in some embodiments, are combined with steps 1-3, and each additional step, or a step on its own, represents a specific embodiment, e.g., see steps 4-8. In some embodiments, barcoded DNA micro-beads are oligonucleotide-capture beads (OCBs). In some embodiments, barcoded DNA micro-beads are capture beads (CBs). In some aspects, beads are loaded into pockets (i.e. ring features) of each chamber.

FIG. 2B is an additional illustration related to the method of manufacture of chip devices (see above), illustrating DNA barcoded micro-beads loaded single-cell chips, and (in some embodiments, 8-cycle barcode reading.

FIG. 2C is an additional illustration related to the method of manufacture of chip devices (see above), illustrating DNA barcoded micro-beads loaded single-cell chips, and (in some embodiments, 4-cycle barcode reading).

FIG. 2D is an illustration depicting a polynucleotide sequence of the DNA barcoded micro-beads. In some embodiments, the polynucleotide sequence comprises any one or more of a split-and-pool nucleotide for cell barcode sequence (J), a unique molecular indicator sequence (N), and a A, C, or G nucleotide (V).

FIG. 2E is an illustration depicting a DNA barcoded micro-bead. In some embodiments, the polynucleotide sequence comprises any one or more of a 5T spacer sequence, a PCR handle sequence, a Unique Molecular Identifier (UMI) sequence, a cell barcode sequence, and a sequencing handle sequence (i.e. barcode handle sequence). In some embodiments, a capture sequence is hybridized to the sequencing handle via priming sequence. In some embodiments, the capture sequences comprise a polyT sequence for mRNA capture. In some embodiments, the capture sequences comprise a sequence-specific or gene-specific capture sequence.

In some embodiments, as shown in FIG. 3A, is a method to perform valve-less lysis of cells on assay chip-devices in single-cell chambers of a substrate each comprising a DNA barcoded micro-bead. In some embodiments, the substrate is unclamped thereby allowing for cell lysis buffer to flow from an inlet port to an outlet port between the substrate and glass slide. In some embodiments, following addition of lysis buffer, the substrate and glass slide are clamped together to isolate each single-cell chambers of the substrate each comprising a DNA barcoded micro-bead with lysis buffer to lyse the single cell.

FIG. 3B is an illustration depicting the assay chip-devices of the disclosure configured for valve-less cell lysis. In some embodiments, the center portion of the device is clamped thereby allowing cell lysis buffer to flow from the inlet port to the outlet port thereby filling the perimeter of the device with cell lysis buffer. In some embodiments, the center of the device is unclamped allowing cell lysis buffer to enter the center portion of the device to lyse cells as described and depicted in FIG. 3A. In some aspects, the unclamping of the center portion creates a gentle and undisruptive flow to allow cells to contact lysis buffer with minimal movement and minimal cell displacement from the chamber to which the cell was originally loaded.

FIG. 4 is an illustration depicting assay chip-devices of the disclosure configured for valve-less cell lysis. In some embodiments, the barcoded micro-beads (in some embodiments, barcoded DNA micro-beads), for loading into assay chip-devices, and in some embodiments, into single-cell chambers of a substrate. In some embodiments, the surface comprises a plurality of substantially parallel lines of capture antibodies for detecting cellular proteins and metabolites. In some embodiments, the surface comprises a fiducial line for aligning the surface and the substrate.

FIG. 5 is an illustration depicting assay chip-devices of the disclosure. In some embodiments, the substrate comprises a plurality of single-cell chambers each configured to retain a single barcoded DNA micro-beads. In some embodiments, the single cell chambers comprise a pocket for retaining the DNA micro-bead wherein the pocket arranged therein in a position offset from a center of the chamber and having a pocket diameter greater than the width of the chamber. In some embodiments, the barcoded DNA micro-beads capture nucleic acids from lysed single cells. In some embodiments, the captured nucleic acids are processed and converted to cDNA.

FIG. 6A is an illustration depicting assay chip-devices of the disclosure configured for valve-less cell lysis. In some embodiments, the barcoded micro-beads (in some embodiments, barcoded DNA micro-beads), for loading into assay chip-devices, and in some embodiments, into single-cell chambers of a substrate. In some embodiments, the surface comprises a plurality of substantially parallel lines of capture antibodies for detecting cellular proteins and metabolites. In some embodiments, the surface comprises a fiducial line for aligning the surface and the substrate.

FIG. 6B is an illustration depicting capture of an mRNA with a barcoded DNA micro-bead by hybridizing the polyA sequence of the mRNA to the polyT sequence of the barcoded DNA micro-bead. In some embodiments, the mRNA is captured from cell lysate. In some embodiments, a captured mRNA is converted to cDNA using reverse transcription. In some embodiments, the cDNA incorporates the barcode sequence, UMI sequence, cell barcode sequence, handle sequence, template switch oligonucleotide (TSO) handle and captured mRNA into a single sequence.

FIG. 6C is an illustration depicting capture of an mRNA with a barcoded DNA micro-bead by hybridizing the polyA sequence of the mRNA to the polyT sequence of the barcoded DNA micro-bead. In some embodiments, the mRNA is captured from cell lysate. I some embodiments, a captured mRNA is converted to cDNA using reverse transcription. In some embodiments, the cDNA incorporates the barcode sequence, UMI sequence, cell barcode sequence, handle sequence, TSO handle and captured mRNA into a single sequence.

FIG. 6D is an illustration depicting nucleic acid library preparation from cDNA. In some embodiments, a PCR handle sequence and TSO handle sequence are used to generate the nucleic acid library. In some embodiments, the gene transcripts and cell barcodes permit spatial determination of where each cell was located on the substrate of the assay chip-device.

FIG. 7A is an illustration depicting assay chip-devices of the disclosure. In some embodiments, the substrate comprises a plurality of single-cell chambers each configured to retain a single barcoded DNA micro-beads. In some embodiments, the single cell single-cell chambers are a pocket for retaining the DNA micro-bead configured to capture sequence-specific nucleic acids wherein the pocket arranged therein in a position offset from a center of the chamber and having a pocket diameter greater than the width of the chamber. In some embodiments, the barcoded DNA micro-beads capture targeted nucleic acids from lysed single cells. In some embodiments, the captured nucleic acids are processed and converted to cDNA.

FIG. 7B is an illustration depicting a DNA barcoded micro-bead. In some embodiments, the polynucleotide sequence comprises any one or more of a 5T spacer sequence, a PCR handle sequence, a UMI sequence, a cell barcode sequence, and a barcode handle sequence. In some embodiments, a capture sequence is hybridized to the barcode handle via priming sequence. In some embodiments, the capture sequences comprise a sequence-specific or gene-specific capture sequence.

FIG. 8A is a schematic depiction of a multiplex assay chip device, according to an embodiment. The multiplex assay chip device can be used for multiplexed analysis of biological material. The multiplex assay chip device includes pocket P(1) P(1) is able to have a capture bead disposed therein.

FIG. 8B is a schematic depiction of a multiplex assay chip device, according to an embodiment. The multiplex assay chip device can be used for multiplexed analysis of biological material. The multiplex assay chip device includes pockets P(1) and P(2). Each of the pockets P(1) and P(2) is able to have a capture bead disposed therein.

FIG. 9 is a schematic depiction of a multiplex as assay chip device, according to an embodiment. The multiplex assay chip device can be used for multiplexed analysis of biological material. The multiplex assay chip device includes pockets P(1), P(2), and P(3).

FIG. 10A is a schematic depiction of a multiplex assay chip device, according to an embodiment. The multiplex assay chip device can be used for multiplexed analysis of biological material. The multiplex assay chip device includes pocket P(1) with capture bead B(1) disposed therein.

FIG. 10B is a schematic depiction of a multiplex assay chip device, according to an embodiment. The multiplex assay chip device can be used for multiplexed analysis of biological material. The multiplex assay chip device includes pockets P(1) and P(2) with capture beads B(1) and B(2) disposed therein.

FIG. 11 is a cross-sectional view of pockets, and various form factors thereof, according to various embodiments.

FIG. 12 is schematic detailing the multiplexed simultaneous detection of a TCR sequence and functional proteomic analysis of cellular proteins such as cytokines in a single T-cell. The device comprises a capture antibody coated surface and a capture bead configured to capture mRNA from the single T-cell.

FIG. 13 is a schematic detailing the functional proteomic analysis of cellular cytokines to derive a polyfunctional strength index (PSI) in T-cells from the process of FIG. 12. For T-cells identified as having a high PSI, the TCR sequence identified in FIG. 12 may be transduced into a T-cell. Following transduction, the T-cell can be expanded and packaged into a final cell product such as an autologous T-cell therapeutic.

FIG. 14 is a schematic detailing the multiplexed simultaneous detection of a BCR sequence and functional proteomic analysis of cellular proteins such as cytokines in a single B-cell. The device comprises a capture antibody coated surface and a capture bead configured to capture mRNA from the single B-cell.

FIG. 15 is a schematic detailing the functional proteomic analysis of cellular cytokines to derive a PSI in B-cells from the process of FIG. 14. For B-cells identified as having a high PSI, the BCR sequence identified in FIG. 14 may be transduced into a B-cell. Following transduction, the B-cell can be expanded and packaged into a final cell product such as an autologous B-cell therapeutic.

FIG. 16 is a schematic detailing multiplexed analysis of determining drug resistance pathways in cancer cells by performing a proteomic and transcriptomic analysis of the cancer cell. Computational analyses are performed to correlate protein expression patterns with transcriptomic data to identify resistance pathways resulting from treatment with the drug. The identified resistance pathways can be selectively targeted with additional drugs to treat the resistant cancer. The device comprises a capture antibody coated surface and a capture bead configured to capture mRNA.

FIG. 17 is a schematic depicting the multiplexed detection of a BCR sequence and cellular protein in a single B-cell using two capture bead devices of the disclosure. The first capture bead is configured to capture mRNA to identify the BCR sequence. The second capture bead is configured to capture cellular proteins.

FIG. 18 is a schematic depicting the multiplexed detection of a BCR sequence and cellular protein in a single B-cell using two capture bead devices of the disclosure. The first capture bead is configured to capture mRNA to identify the BCR sequence. The second capture bead is configured to capture cellular proteins. The device further includes an antibody coated capture surface for performing further proteomic analysis of the B-cell.

FIG. 19 is a schematic depicting the multiplexed detection of an mRNA sequence, an additional RNA or DNA sequence, and a cellular protein in a single cell using a two-capture bead device of the disclosure. The first capture bead is configured to capture mRNA sequences. The second capture bead is configured to capture the additional RNA or DNA sequence. The antibody coated capture surface is configured to perform further proteomic analysis of the cell.

FIG. 20 is a schematic detailing the creation of a nucleic acid capture bead of the disclosure. 1) A bead comprising at least one nucleic acid sequence comprising from 5′ to 3′ a 5T spacer sequence, a PCR handle sequence, a unique molecular identifier (UMI) sequence, cell barcode sequence, and sequencing (seq) handle. 2) The cell barcode sequence can be read by hybridizing a barcode reading primer to the seq handle sequence and extending the primer using the cell barcode sequence as a template. Sequencing the cell barcode can be performed “on chip” or on substrate using fluorescently labeled nucleotides and a fluorescent imaging system. 3) In some aspects, a capture sequence can be added to the capture bead nucleic acid sequence by hybridizing an oligonucleotide sequence comprising a capture sequence to the sequence handle and extending the sequence using PCR to produce 4) bead comprising at least one nucleic acid sequence comprising from 5′ to 3′ a 5T spacer sequence, a PCR handle sequence, a unique molecular identifier (UMI) sequence, cell barcode sequence (individually unique cell barcode), a sequencing (seq) handle, and a capture sequence.

FIG. 21 is an image of a capture bead configured to capture mRNA sequences by hybridizing a polyT capture sequence to the 3′ polyA sequence of an mRNA.

FIG. 22 is a schematic detailing 5′ mRNA capture using capture beads of the disclosure configured to comprise rGrGrG (three riboguanosines) for capture of 5′ mRNA sequences that have been reverse transcribed to include a terminal polyC sequence.

FIG. 23 is an image of a capture bead configured to capture nucleic sequences by hybridizing a sequence specific capture sequence a portion of a target nucleic acid such as an RNA or DNA sequence. In some aspects, this is a gene-specific capture sequence.

FIG. 24 is a heat map of protein expression and hierarchical cluster of 73 single Hela and monocyte cells from the cell sample. Each column of the map represents a single cell. Cells were matched to their location based on the bead cell barcode sequence. High protein is indicated in red while low protein expression is indicated in blue. Each protein is indicated in a separate row.

FIG. 25 is a heat map of protein expression and hierarchical cluster of 45 single Hela and monocyte cells from the cell sample. Each column of the map represents a single cell. Cells were matched to their location based on the bead cell barcode sequence. High protein is indicated in red while low protein expression is indicated in blue. Each protein is indicated in a separate row.

FIG. 26 is a heat map of raw gene expression and hierarchical cluster of 45 single Hela and monocyte cells from the cell sample. Each column of the map represents a single cell. Cells were matched to their location based on the bead cell barcode sequence. High gene expression is indicated in red while low gene expression is indicated in blue. Each gene is indicated in a separate row.

FIG. 27 is a heat map of normalized and logged gene expression and hierarchical cluster of 45 single Hela and monocyte cells from the cell sample. Each column of the map represents a single cell. Cells were matched to their location based on the bead cell barcode sequence. High gene expression is indicated in red while low gene expression is indicated in blue. Each gene is indicated in a separate row.

FIG. 28A is UMAP (Uniform Manifold Approximation and Projection) applied to the gene expression data for the 45 matched cells.

FIG. 28B is a UMAP applied to the gene expression data for the 45 matched cells. UMAP is a dimension reduction technique that allows for visualization of variance between data points. The cells are shaded by the number of genes detected per cell with lighter shading being indicating a larger number of genes/cells and darker shading indicating a lower number of genes/cell.

FIG. 29 is a heat map of the top 50 genes for each cluster, subgroup 0 and subgroup 1. This heat map displays the expression of top 50 marker genes for each of the two clusters calculated by the leiden algorithm. Higher expression is denoted by lighter shading while lower expression is denoted by darker shading. The 100 columns represent the respective top 50 marker genes for each of the two clusters. The rows represent the cells and are color coded by which cluster they were calculated to be from.

FIG. 30 is a heat map and hierarchical cluster of the top 1000 most variable genes. Subgroup 0 (cluster 0) is shaded with diagonal lines and subgroup 1 (cluster 1) is shaded. High gene expression is indicated in red while low gene expression is indicated in blue.

FIG. 31A is a violin plot of RPS6 gene expression for subgroup 0 and subgroup 1. Each dot represents a unique cell from our matched 45 cells.

FIG. 31B is a violin plot of MIP-1-Beta gene expression for subgroup 0 and subgroup 1. Each dot represents a unique cell from the 45 matched cells.

FIG. 31C is a violin plot of beta actin gene expression for subgroup 0 and subgroup 1. Each dot represents a unique cell from the 45 matched cells.

FIG. 32A is a 2D t-Distributed Stochastic Neighbor Embedding (t-SNE) transformation plot of single melanoma cells subject to multiplexed phosphoprotein expression analysis.

FIG. 32B is a series of 2D t-SNE plots for each phosphoprotein. Protein intensity is indicated by color, with low intensity being blue and high intensity indicated in red. Analyzed proteins include: alpha tubulin, cleaved PARP, P-elF4E, P-IkBA, P-MEK1-2, P-Met, P-NF-kB p65, P-p44-42 MAPK, P-p90RSK, P-PRAS40, p-Rb, P-s6 Ribosomal, p-Stat 1, p-Stat 3, and p-Stat 5.

FIG. 33A is a graph depicting the heterogeneity of each subset of cells from FIG. 32A.

FIG. 33B is graph depicting the functional heterogeneity index of each subset of cells from FIG. 32A.

FIG. 34 is a heat map and hierarchical cluster with statistically significant differences in protein expression between the two identified cell subsets. Each column of the map represents a single cell from the samples in FIG. 32A. **p<0.01; ***p<0.001; and ****p<0.0001.

FIG. 35 is a heat map and hierarchical cluster with statistically significant differences in gene expression between the two identified cell subsets. Each column of the map represents a single cell from the samples in FIG. 32A. *All displayed genes had differential expression between the two cell subsets (p value<0.05).

FIG. 36 is a series of 2D t-SNE plots for key genes with statistically differential expression between these two subsets, color-coded by the given gene's expression intensity values. Gene expression intensity is indicated by shade intensity, with low intensity being shaded darker and high intensity shaded lighter. *All displayed genes had differential expression between the two cell subsets (p value<0.01).

FIG. 37A is a reactome pathway analysis demonstrating that some of the significantly up-regulated genes in cell subset 2 (high functional heterogeneity) observed to be significantly involved in pathways directly responsible for cellular metabolism, showing correlation with higher functional heterogeneity from protein expression.

FIG. 37B is a table depicting the pathways identified in the reactome pathway analysis. Highlighted pathways are involved in cellular metabolism and include translation, mitochondrial translation termination, mitochondrial translation elongation, mitochondrial translation initiation, and mitochondrial translation.

FIG. 38 is a combined heat map of the cells comprising subset 1 and subset 2 depicting the proteins from FIG. 34 and the genes from FIG. 35. Each column of the map represents a single cell. High protein expression is indicated by red shading while low protein expression is indicated by blue shading. High gene expression is indicated by red shading while low gene expression is indicated by blue shading. *All displayed genes and proteins had differential expression between the two cell subsets (p value<0.05).

FIG. 39 is a table detailing proteins with statistically significant differences between the two cell populations, subset 1 and subset 2.

### DETAILED DESCRIPTION

### Definitions

Unless otherwise defined, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The following definitions are useful in understanding the present invention:

The term “antibody” (Ab) as used herein includes monoclonal antibodies, polyclonal antibodies, multispecific antibodies (e.g., bispecific antibodies), and antibody fragments, as long as they exhibit the desired biological activity. The term “immunoglobulin” (Ig) is used interchangeably with “antibody” herein. In some aspects, the antibody is an isolated antibody.

The term “about”, “approximately”, or “approximate”, when used in connection with a numerical value, means that a collection or range of values is included. In some embodiments, “about X” includes a range of values that are ±25%, ±20%, ±15%, ±10%, ±5%, ±2%, ±1%, ±0.5%, ±0.2%, or ±0.1% of X, where X is a numerical value. In some embodiments, the term “about” refers to a range of values which are 5% more or less than the specified value. In some embodiments, the term “about” refers to a range of values which are 2% more or less than the specified value. In some embodiments, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. In some embodiments, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Capture antibodies of the disclosure may comprise one or more monoclonal antibodies. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

Monoclonal antibodies contemplated herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Capture antibodies of the disclosure may comprise humanized antibodies. A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed by substituting import hypervariable region sequences for the corresponding sequences of a human antibody. Accordingly, such “humanized” antibodies are chimeric antibodies wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

Capture antibodies of the disclosure may comprise intact antibodies. An “intact” antibody is one that comprises an antigen-binding site as well as a CL and at least heavy chain constant domains, CH 1, CH 2 and CH 3. The constant domains may be native sequence constant domains (e.g., human native sequence constant domains) or amino acid sequence variant thereof. Preferably, the intact antibody has one or more effector functions.

Capture antibodies of the disclosure may comprise an antibody fragment. An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments.

Capture antibodies of the disclosure may comprise a functional fragment or an analog of an antibody. The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, FcεRI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH 1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Capture antibodies of the disclosure may comprise single-chain antibodies (also referred to as scFv). “Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

Capture antibodies of the disclosure may comprise diabodies. The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

Capture antibodies of the disclosure may comprise bispecific antibodies. In certain embodiments, antibodies of the present invention are bispecific or multi-specific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen.

Methods for making bispecific antibodies are known in the art. Traditional production of full length bispecific antibodies is based on the co-expression of two immunoglobulin heavy chain-light chain pairs, where the two chains have different specificities. Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low.

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant KD, and in certain embodiments, an antibody specifically binds to a component of a secretome if it binds with a KD of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Subject and target cells of the disclosure may be isolated, derived, or prepared from any species, including any mammal. A “mammal” for purposes of treating n infection, refers to any mammal, including humans, domestic and farm animals, and zoo, sports, or pet animals, such as dogs, cats, cattle, horses, sheep, pigs, goats, rabbits, etc. Preferably, the mammal is human.

Subject cells of the disclosure may be used in a cellular therapy for the treatment of a disease or disorder. “Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal may be successfully “treated” when, after receiving a cellular therapy with a subject cell of the disclosure, the patient shows observable and/or measurable reduction in or absence of one or more of the following: reduction in one or more of the symptoms associated with disease or disorder; reduced morbidity and mortality, and improvement in quality of life issues. The above parameters for assessing successful treatment and improvement in the disease are readily measurable by routine procedures familiar to a physician. Methods of the disclosure may be used to determine the safety and/or efficacy of a cellular therapy before, during or after initiation of treatment of the subject.

Capture antibodies of the disclosure may be labeled to render them detectable using one or more means. “Label” as used herein refers to a detectable compound or composition that is conjugated directly or indirectly to the capture antibody so as to generate a “labeled” capture antibody. The label may be detectable by itself (e.g., a fluorescent label) or, in the case of an enzymatic label, may catalyze chemical alteration of a substrate compound or composition that is detectable.

Capture antibodies of the disclosure may selectively or specifically identify, capture, and/or quantify one or more small molecules in a secretome. A “small molecule” is defined herein to have a molecular weight below about 500 Daltons.

Capture antibodies of the disclosure may include nucleic acids or labeled nucleic acids. The terms “nucleic acid” and “polynucleotide” are used interchangeably herein to refer to single- or double-stranded RNA, DNA, or mixed polymers. Polynucleotides may include genomic sequences, extra-genomic and plasmid sequences, and smaller engineered gene segments that express, or may be adapted to express polypeptides.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated polypeptide includes the polypeptide in situ within recombinant cells since at least one component of the polypeptide's natural environment will not be present. Ordinarily, however, isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means.

A polynucleotide “variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the invention and evaluating one or more biological activities of the encoded polypeptide as described herein and/or using any of a number of techniques well known in the art.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating one or more biological activities of the polypeptide as described herein and/or using any of a number of techniques well known in the art.

Modifications may be made in the structure of the polynucleotides and polypeptides of the disclosure and still obtain a functional molecule that encodes a variant or derivative polypeptide with desirable characteristics. When it is desired to alter the amino acid sequence of a polypeptide to create an equivalent, or even an improved, variant or portion of a polypeptide of the invention, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences that encode said peptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. The substitution of like amino acids can be made effectively on the basis of hydrophilicity. The greatest local average hydrophilicity of a protein, as governed by the hydrophilicity of its adjacent amino acids, correlates with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, CA; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information.

“Homology” refers to the percentage of residues in the polynucleotide or polypeptide sequence variant that are identical to the non-variant sequence after aligning the sequences and introducing gaps, if necessary, to achieve the maximum percent homology. In particular embodiments, polynucleotide and polypeptide variants have at least 70%, at least 75%, at least 80%, at least 90%, at least 95%, at least 98%, or at least 99% polynucleotide or polypeptide homology with a polynucleotide or polypeptide described herein.

As used in this specification and the appended claims, the singular forms “a,” “an” and “the” include plural references unless the content clearly dictates otherwise.

In some embodiments, the disclosure provides a multiplex assay chip device configured for multiplexed analysis of biological material, the device comprising a plurality of capture beads (CB), each CB having an individually unique cell barcode sequence comprising a predetermined number of base pairs and a PCR handle, a unique molecular identifier, a barcode handle sequence, and a poly T sequence/capture sequence, a substrate having a plurality of chambers, wherein each chamber includes an open end arranged on a first side of the substrate, and a length, a width, and a depth (“dimensions”), a pocket arranged therein having a pocket diameter greater than the width of the chamber, wherein the at least one pocket is alternately arranged and offset from at least one pocket of an adjacent chamber and at least one of the OCBs arranged within the pocket of each chamber, and each CB including an CB diameter smaller than the pocket diameter but larger than the width of the chamber, such that each pocket and each CB are configured such that the CB is arranged approximately in the center of each chamber, and a surface configured to couple with the first side of the substrate to cover each chamber, wherein the surface comprising a plurality of substantially parallel lines of capture antibodies, each line of capture antibodies comprises a specific and different antibody, relative to antibodies of adjacent lines of capture antibodies, configured to bind to a different target molecule, and at least one portion of each of the plurality of lines are arranged as to be exposed to each chamber. In some embodiments, the capture bead can also be referred to as an oligonucleotide capture bead.

In some embodiments, the disclosure provides a multiplex assay chip device manufacturing method comprising providing a plurality of CBs, each CB having an individually unique cell barcode sequence comprising a predetermined number of base pairs and a PCR handle, a unique molecular identifier, a barcode handle sequence, and a poly T sequence/capture sequence, providing a chip substrate having a plurality of chambers, each chamber includes an open end arranged on a first side of the substrate, and a length, a width, and a depth (“dimensions”), a pocket arranged therein having a pocket diameter greater than the width of the chamber, loading at least one of the CBs, and preferably, a single CB, within each chamber and preferably, within the pocket of each chamber, wherein each CB including an CB diameter smaller than the pocket diameter but larger than the width of the chamber, such that, each pocket and each CB are configured such that the CB is arranged approximately in the center of each chamber, coupling a chip surface to the first side of the substrate to cover each chamber, wherein the surface comprising a plurality of substantially parallel lines of capture antibodies, each line of capture antibodies comprises a specific and different antibody, relative to antibodies of adjacent lines of capture antibodies, configured to bind to a different target molecule, at least one portion of each of the plurality of lines are arranged as to be exposed to each chamber, and the combination of the chip surface and chip substrate form an assay chip device, imaging the chip such that each unique cell barcode sequence of each CB is read and to obtain data detailing the location of each CB/pocket on the chip, and storing the data.

In some embodiments, the disclosure provides a capture-bead barcode sequence determination method for determining the barcode sequences on CBs immobilized on a chip device comprising providing the devices of any preceding embodiment, obtaining the sequence of the cell barcode sequence of the oligonucleotide of each OCB and correlating the sequence of the cell barcode sequence of each CB to a spatial location of each respective open rectangular chamber to produce a spatial map of the chip device.

In some embodiments, loading the CBs into the chambers comprises immobilizing the CBs therein. In some embodiments, loading the CBs into the chambers comprises providing the CBs within a first buffer solution, placing the chip device onto a rocker device, loading the buffer into a flow cell, such that one CB settles within each pocket via at least one of gravity and pocket diameter and relative CB diameter, wherein the rocker device is configured to encourage an CB to load into each pocket, washing excess beads from the chip device via a second buffer solution, drying the chip device at a first predetermined temperature (in some embodiments, 60 deg. C.), heating a agarose material (in some embodiments, a 0.15% agarose material) to dissolve in TBE buffer, transferring the agarose material (e.g., via pipetting) at a predetermined second temperature lower than the first predetermined temperature, spin-coating the chip device via centrifugation at increasing speeds for a first predetermined period of time (e.g., in some embodiments, under 2 minutes), and cooling the agarose material at a third predetermined temperature (e.g., room temperature), such that the agarose material gels so as to immobilize each CB in each respective chamber, and gelling occurs within a predetermined time period (e.g., in some embodiments, greater than 1 hour).

In some embodiments, the disclosure provides a device for loading CBs into pockets of an assay chip device of any preceding embodiment, the flow cell comprising a clamp comprising a first side and a second side, a housing corresponding to the second side of the clamp and configured to retain the chip surface (e.g., glass slide) at a first side of the housing (and optionally comprising the first side of the housing), and retain the chip substrate in a spaced apart arrangement from the chip surface, a void therebetween configured as a flow area, an inlet port arranged on a first end of the housing configured to receive a solution containing a plurality of biological cells, the inlet port being in fluid communication with a first end of the flow area, and an outlet port arranged on a second end of the housing and configured to exhaust the solution from the flow area, the outlet port being in fluid communication with a second end of the flow area, wherein upon the solution flowing through the flow area, whereby individual biological cells are distributed adjacent a plurality of the chambers (in some embodiments, all the chambers), and wherein the clamp is configured such that the second side is advanced toward the first side, driving individual biological cells into respective pockets on the substrate, and wherein the chip surface is coupled to the chip substrate.

In some embodiments, including in certain embodiments described above, the disclosure provides a device for performing cell lysis to a plurality of individual cells arranged in a plurality of respective pockets of an assay chip device comprising a clamp comprising a first side and a second side, a housing corresponding to the second side of the clamp and configured to retain the chip surface (e.g., glass slide) at a first side of the housing (and optionally comprising the first side of the housing) and retain the chip substrate in a spaced apart arrangement from the chip surface, a void therebetween configured as a flow area, the flow area including a central portion and a perimeter portion surrounding a perimeter of the chip device, an inlet port arranged on a first end of the housing configured to receive a lysis solution, the inlet port being in fluid communication with a first end of the flow area, and an outlet port arranged on a second end of the housing and configured to exhaust the lysis solution from the flow area, the outlet port being in fluid communication with a second end of the flow area, when the clamp is in a first configuration, the chip surface is coupled to the chip substrate and the central portion of the flow area is inaccessible, the lysis solution flowing into the inlet port flows into the perimeter portion of the flow area and toward the outlet port, thereby filling the perimeter portion to a predetermined amount, and when the clamp is in a second configuration, the chip surface is decoupled from the chip substrate, the central portion of the flow area is accessible, and the lysis solution from the perimeter portion of the flow area diffuses into the central portion of the flow area, thereby allowing cells in each chamber to contact the lysis solution.

In some embodiments of the disclosure, a method of on-chip complementary DNA (cDNA) processing is provided which includes providing a flow-cell chip having a plurality of capture beads (CBs), where each is configured to capture RNA, a substrate having a plurality of chambers, where each chamber includes at least one of the CBs arranged therein, and a surface configured to couple with the first side of the substrate to cover each chamber. The surface optionally comprises a plurality of substantially parallel lines of capture antibodies, where the surface is removably coupled to the substrate, such that the chip includes an open configuration, and a closed configuration. Opening the chip corresponds to causing the chip to proceed from the closed configuration to the open configuration, and closing the chip corresponds to causing the chip to proceed from the open configuration to the closed configuration, In the open configuration, materials can flow into and/or out of the chambers. The method further includes providing a single cell in each of a plurality of the chambers, lysing the single cell within the plurality of the chambers, while the chip is in the closed configuration, so as to release RNA, opening the chip and directing one or more reagents into the plurality of chambers configured for a reverse transcription reaction, closing the chip such that in the closed configuration, RNA reverse transcription occurs forming cDNA, removing the cDNA from each of the plurality of chambers, and opening the chip and removing the cDNA from the chip.

In some embodiments, the CBs configured to capture RNA comprise a poly T sequence.

In some embodiments, the one or more reagents for the reverse transcription reaction comprise a deoxyribonucleotide triphosphate mixture comprising adenine, guanine, cytosine, and thymine, a reverse transcriptase, and a reverse transcription primer.

In some embodiments, the one or more reagents for the reverse transcription reaction further comprise Ficoll, and one or more reverse transcription buffers.

In some embodiments, RNA reverse transcription comprises hybridizing the reverse transcription primer to a reverse transcription hybridization site on the CB.

In some embodiments, wherein RNA reverse transcription comprises a thermal cycling step.

In some embodiments, the disclosure provides a multiplexed proteomic and nucleic acid identification method comprising providing the chip device of any preceding embodiment, mapping chambers of the chip device via the process of any preceding embodiment so as to form a map of the chip device and the location of each CB, storing the map, loading a single cell within each of the plurality of chambers of the chip device, lysing the single cell in each chamber such that the single cell releases a plurality of cellular components comprising nucleic acid(s), and at least one of one or more cellular proteins and one or more target molecules of the plurality of cellular components, identifying at least one of the cellular proteins and target molecules by providing reaction conditions configured to enable the formation of at least one antibody complex from respective interactions between at least one of the cellular proteins and at least one capture antibody from the plurality of lines of capture antibodies, or, at least one target molecule of the plurality of cellular components and the at least one capture antibody from the plurality of lines of capture antibodies, providing at least one labeled secondary antibody that specifically binds to the at least one capture antibody complex to provide a detectable signal thereof, the detectable signal comprising a signature of the protein or target molecule forming the antibody complex, identifying the nucleic acid by providing reaction conditions configured to enable nucleic acid to bind with the capture sequence of the CB forming one or more respective captured nucleic acids, preparing cDNA of the captured cellular nucleic acids comprising the steps of performing reverse transcription of the one or more respective captured cellular nucleic acids comprising hybridizing a reverse transcription primer to the PCR handle of the CB, wherein each cDNA comprises the cell barcode sequence of the CB, removing cDNA from individual CBs, amplifying cDNA to form a cDNA library by performing PCR, sequencing cDNA library, spatially locating each cDNA via the map.

This disclosure provides devices and methods capable of analyzing multiple cellular activities and pathways in single cells in a high-throughput format wherein hundreds of intracellular components, including DNA, RNA, and protein levels of each single cell of a plurality of thousands of cells are analyzed in parallel. In some aspects, methods of the disclosure enable the development of novel therapeutics for treating immune diseases and cancers. In some aspects, devices of the disclosure are configured to simultaneously detect transcriptomic, proteomic, and genomic information from a single cell.

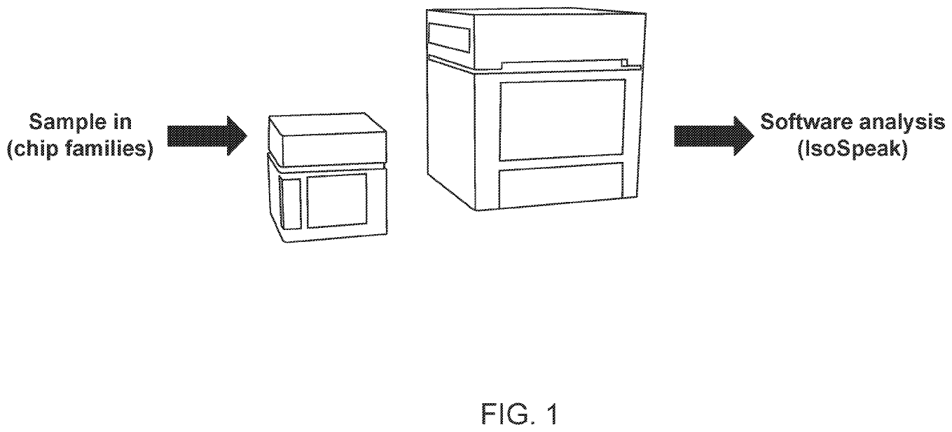

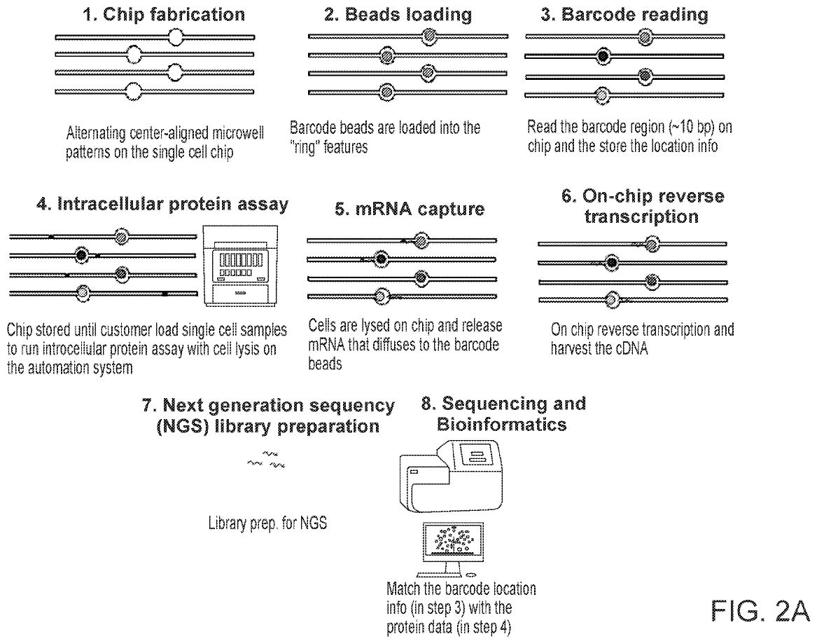

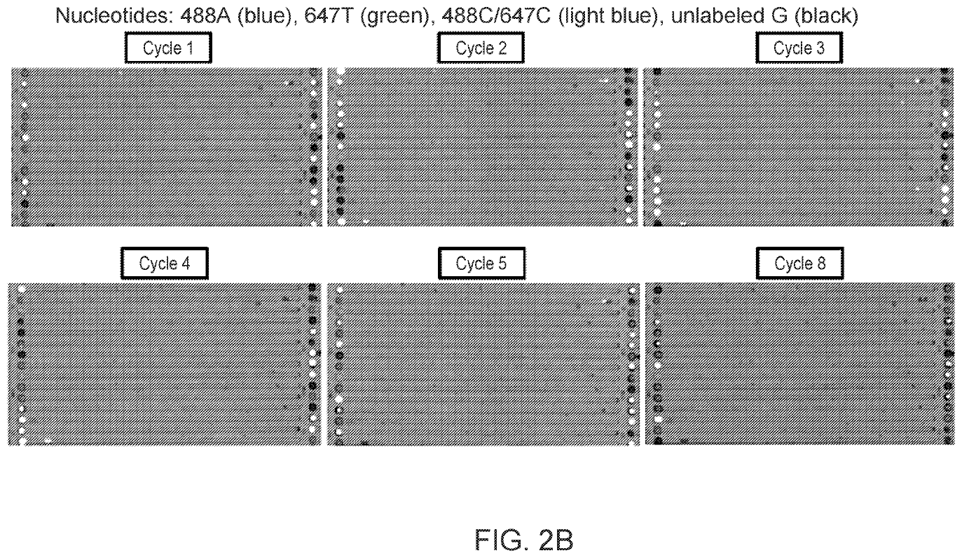

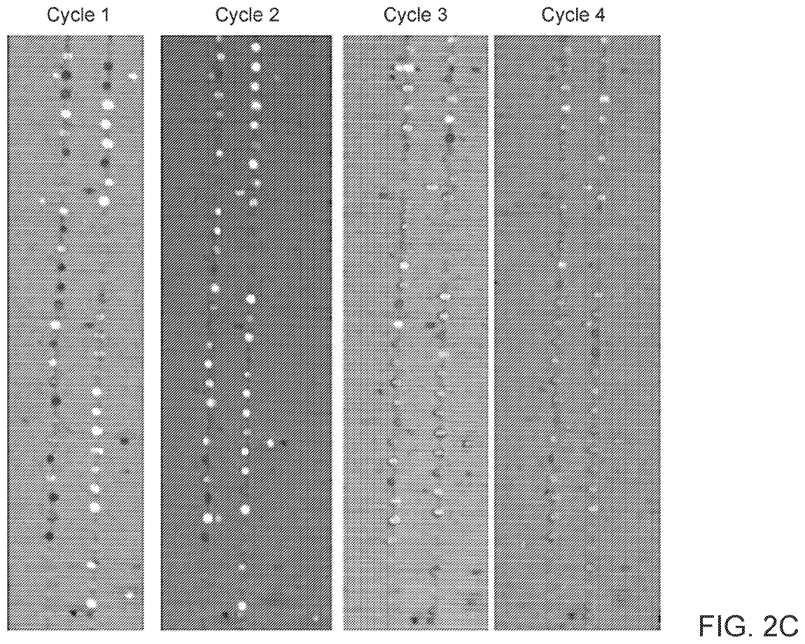

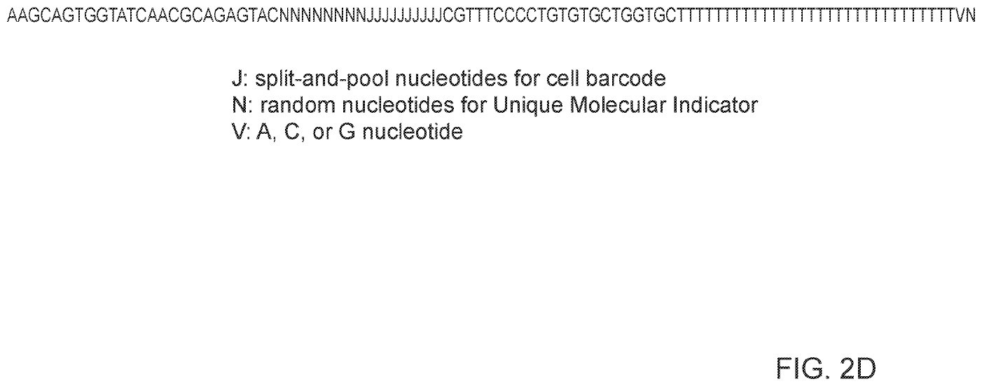

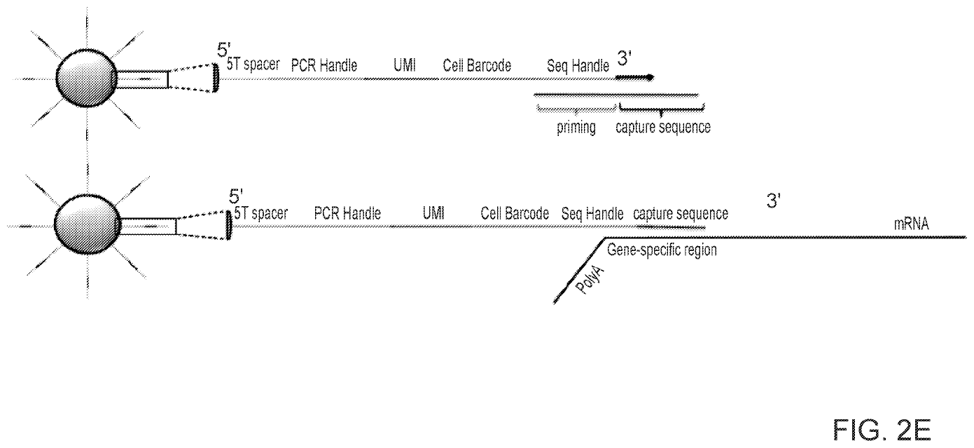

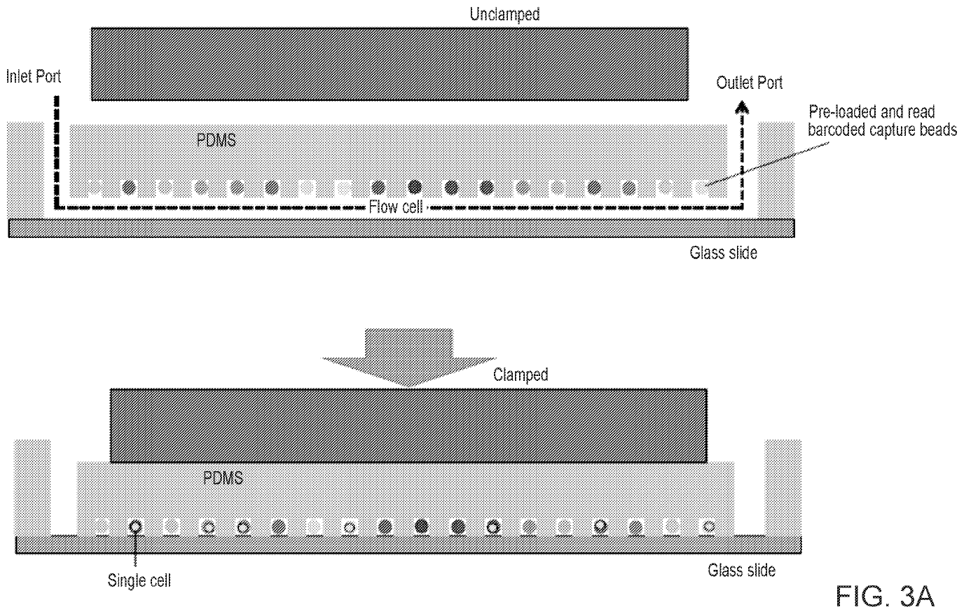

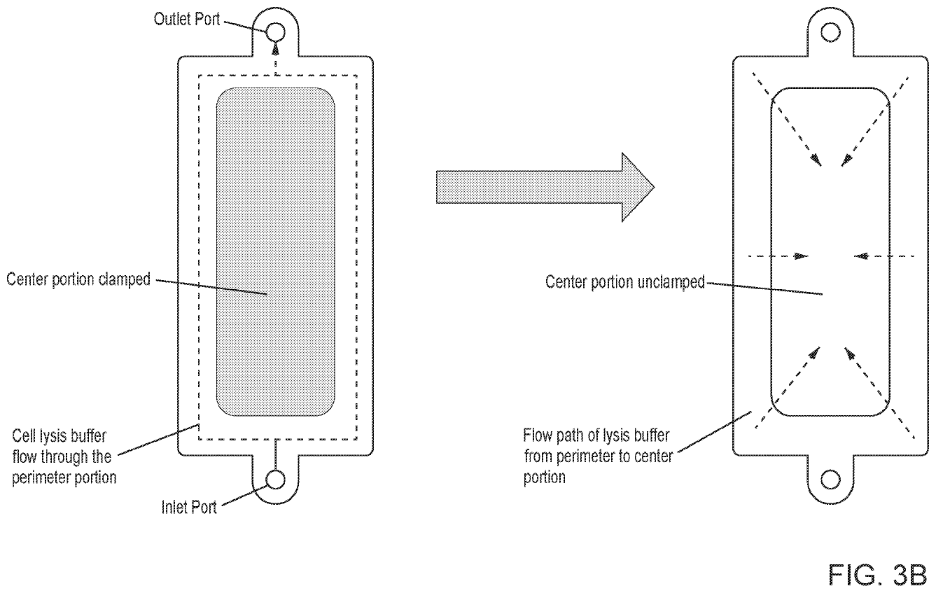

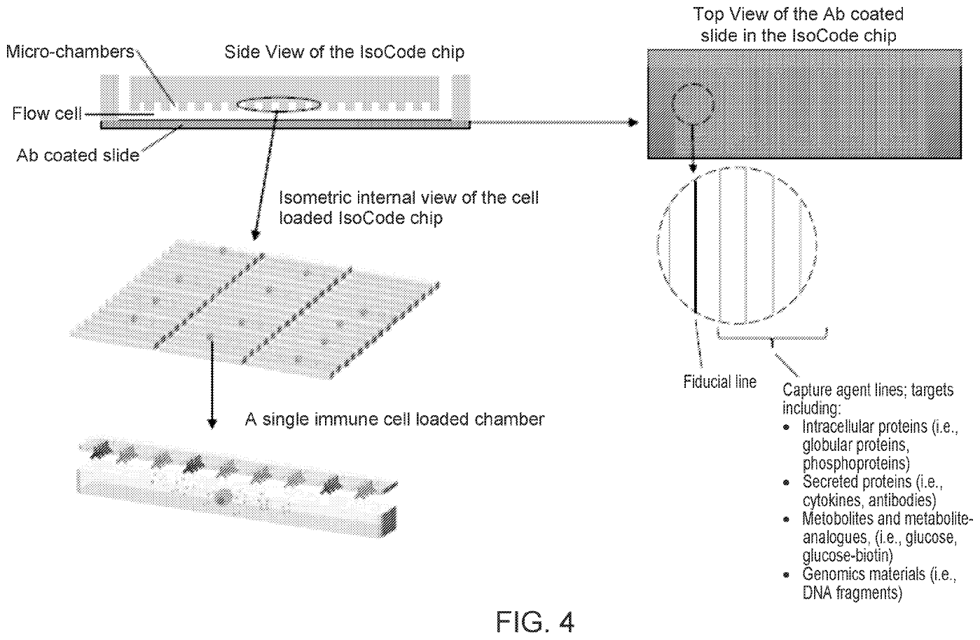

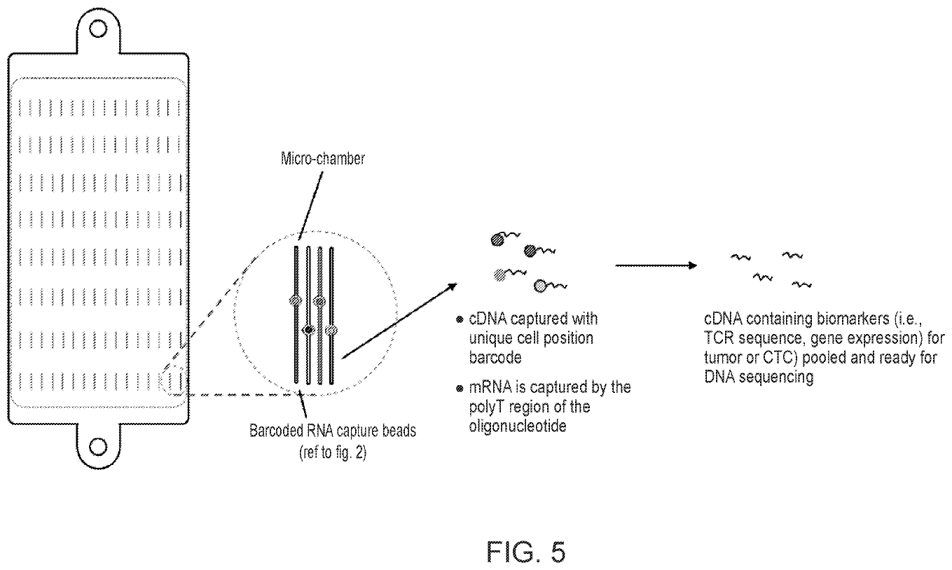

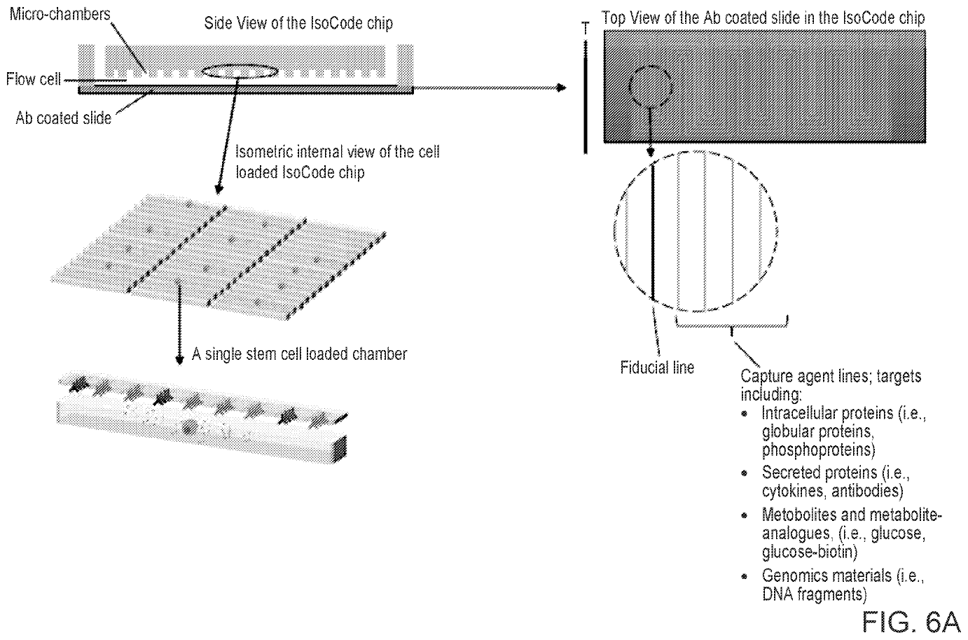

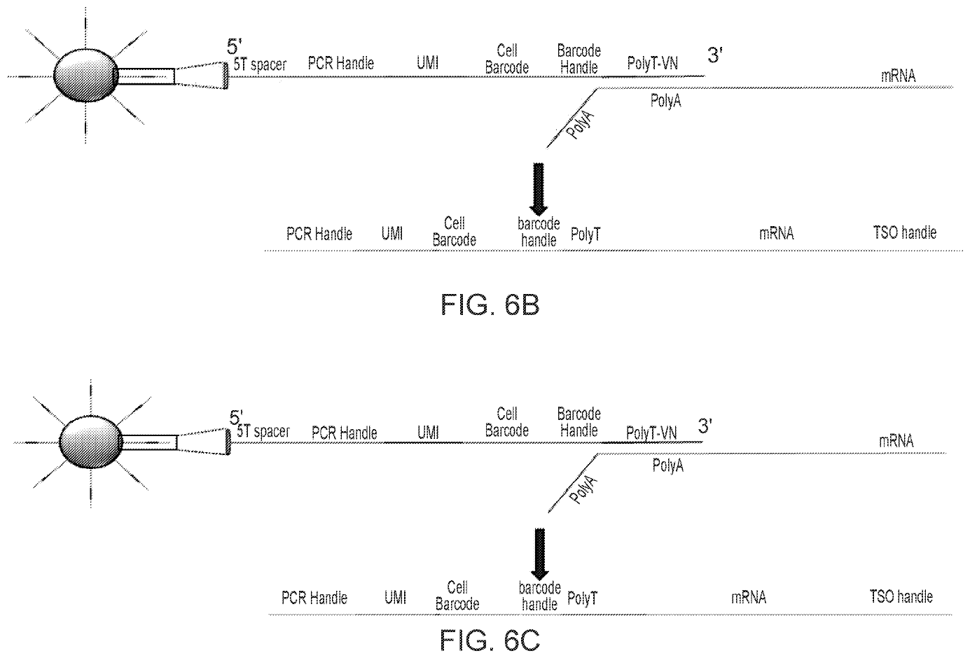

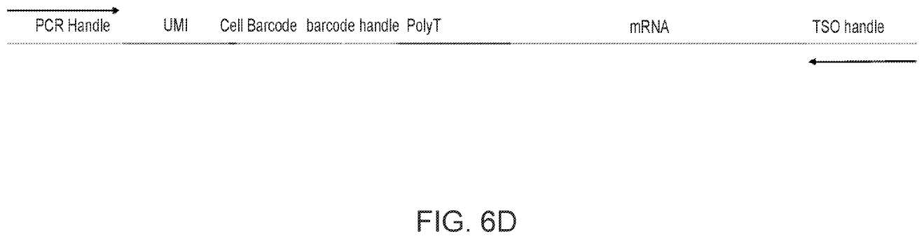

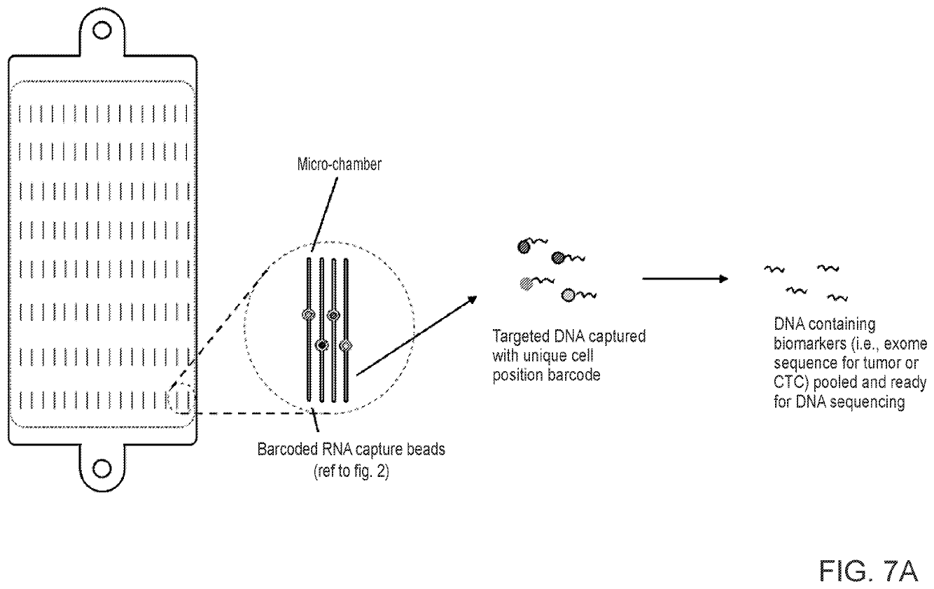

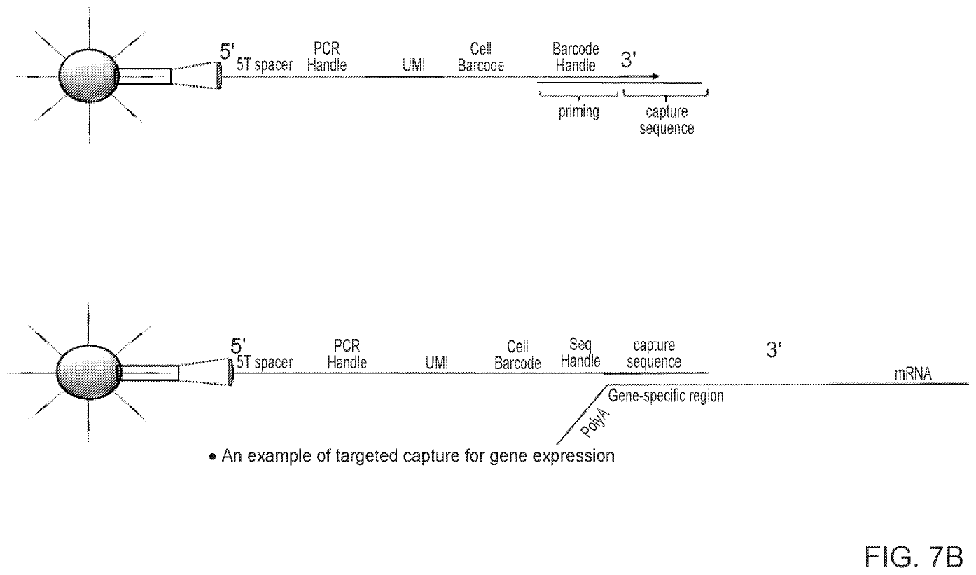

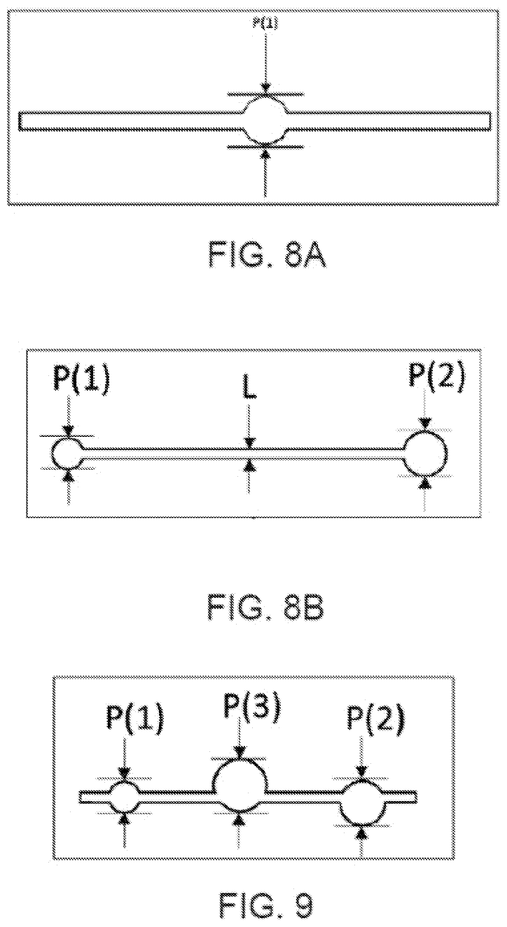

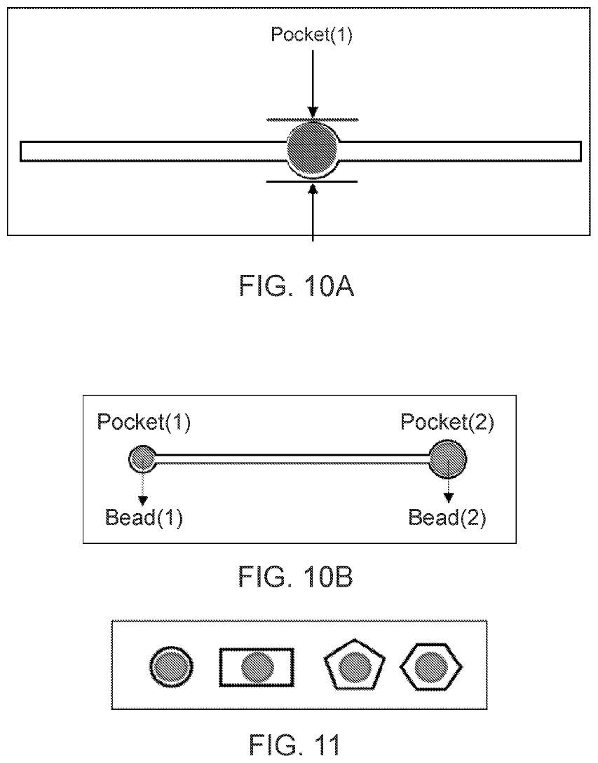

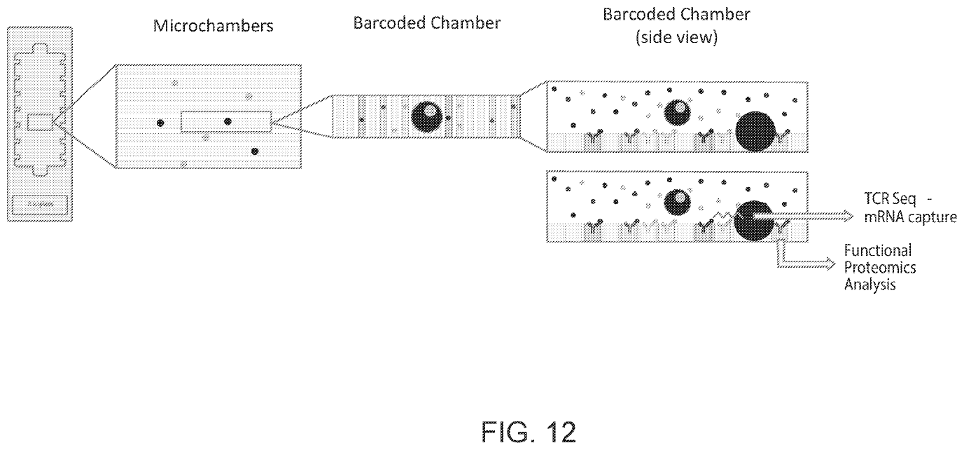

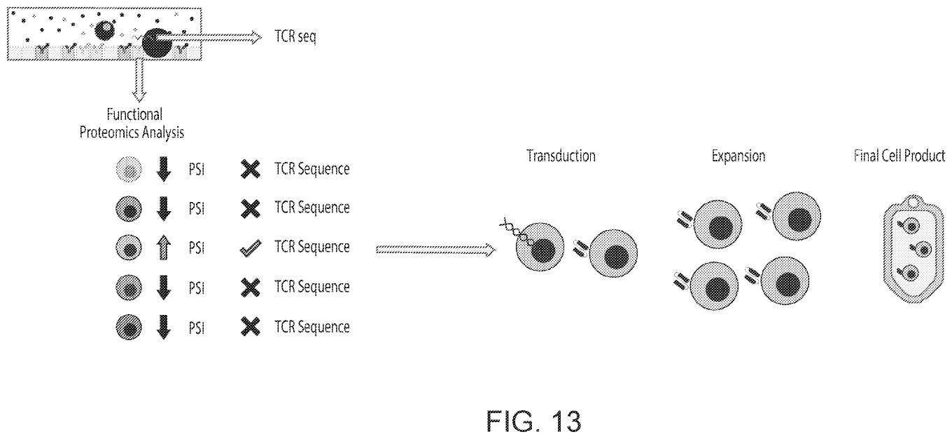

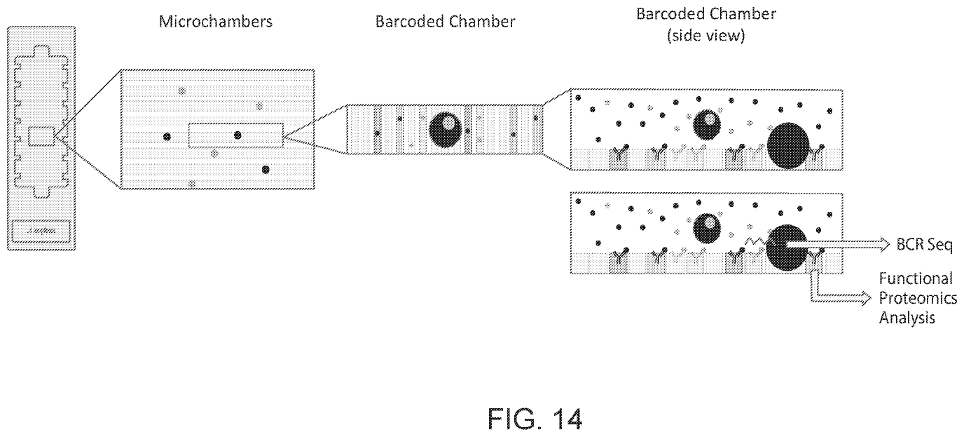

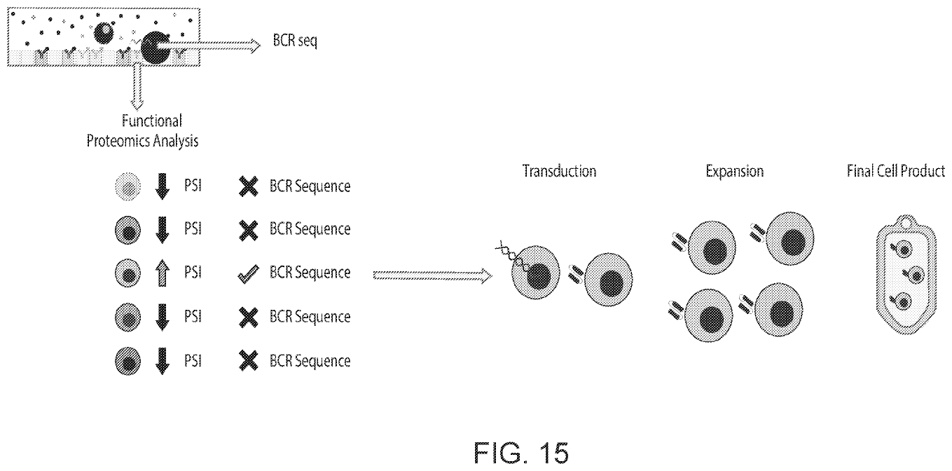

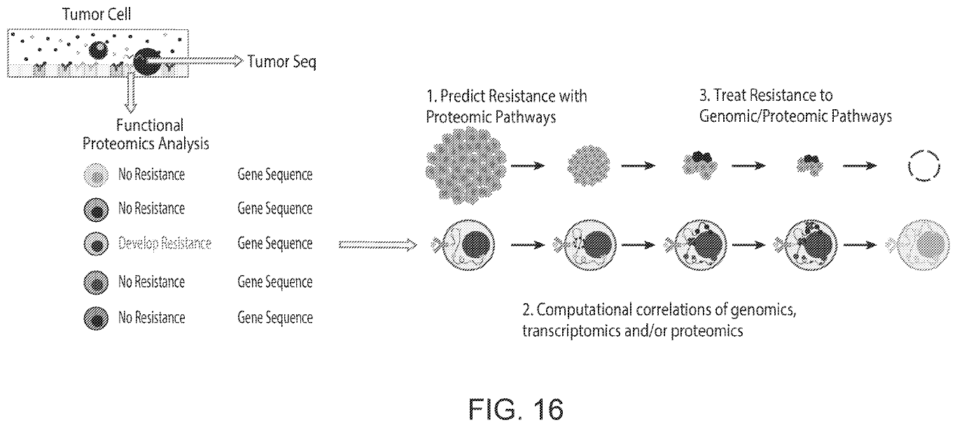

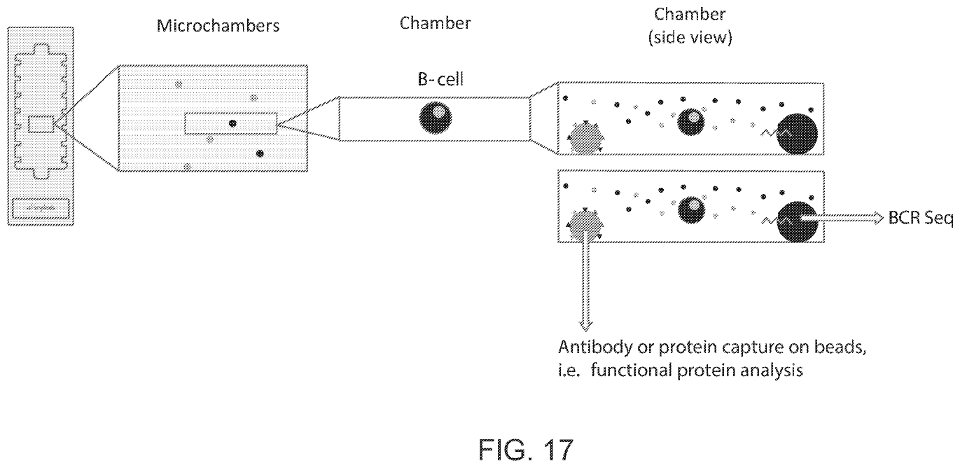

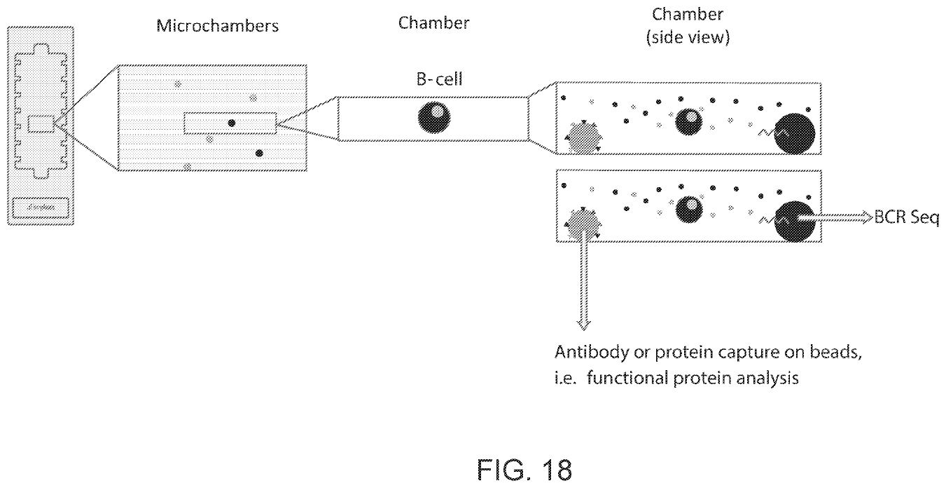

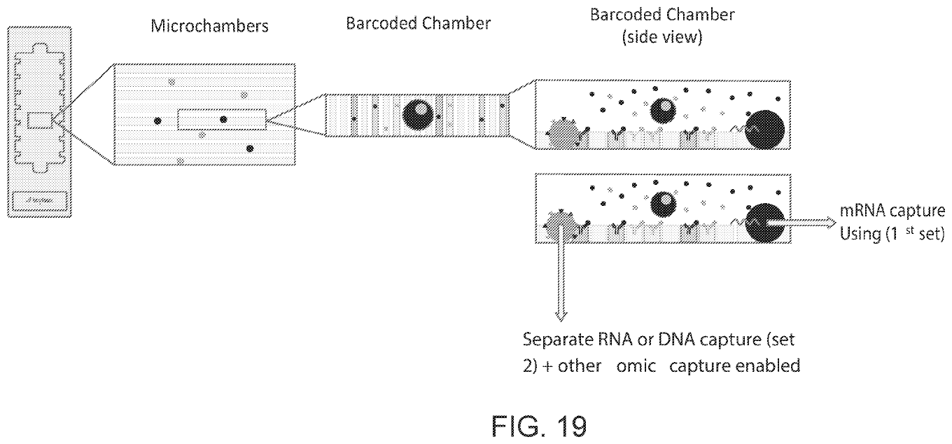

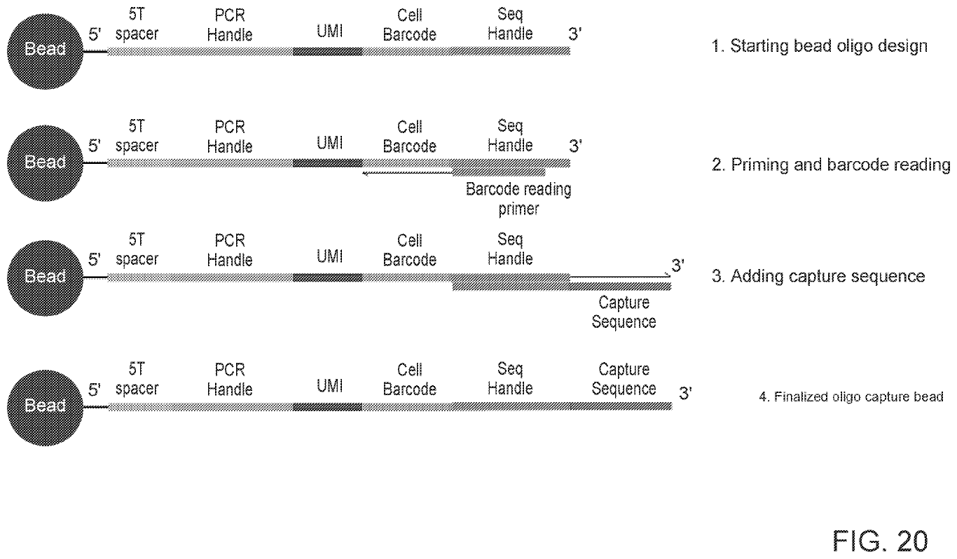

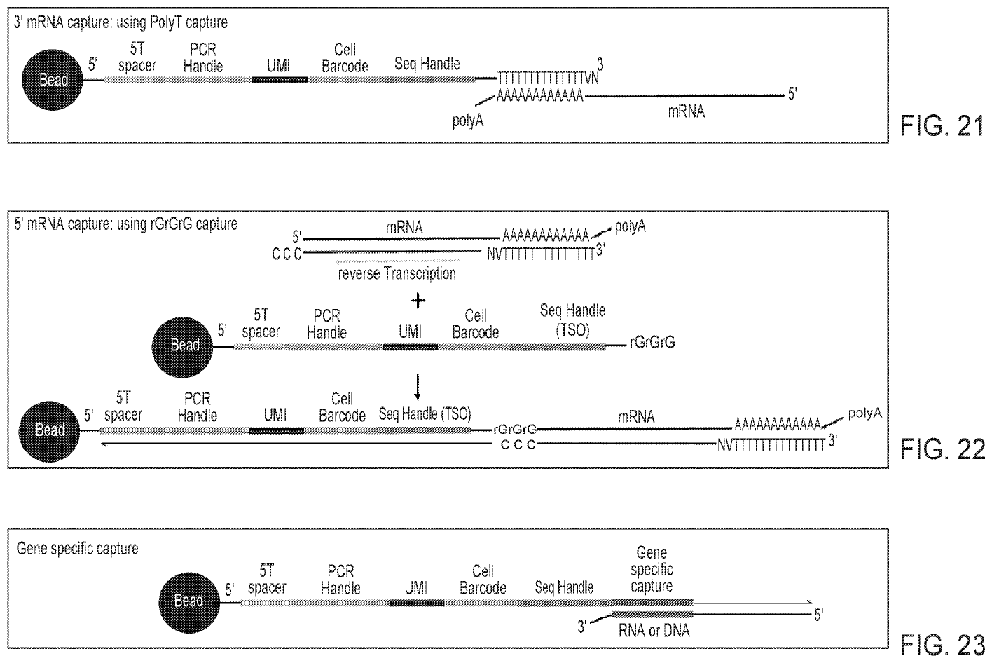

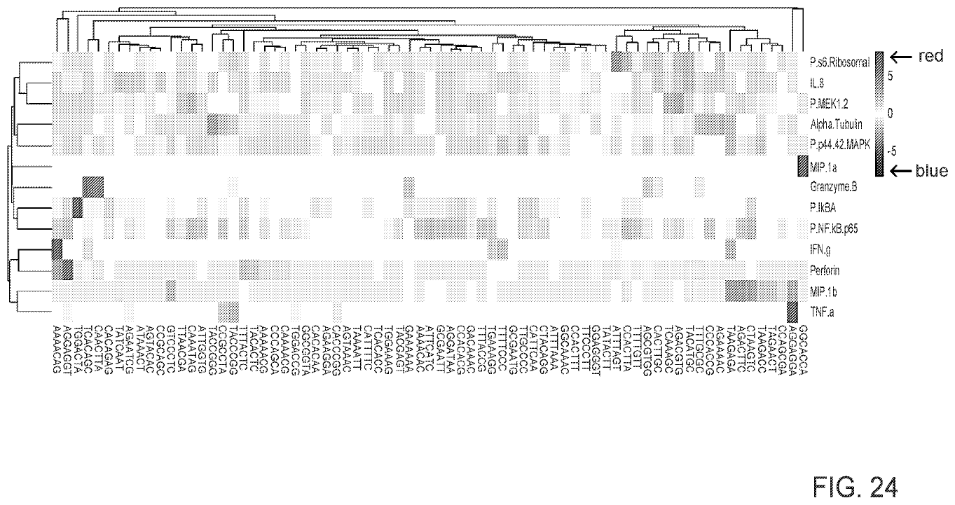

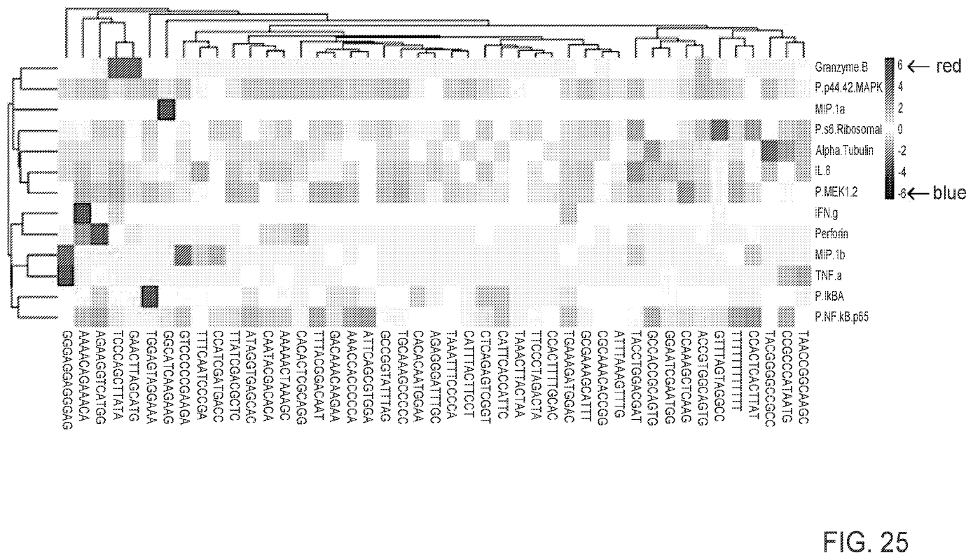

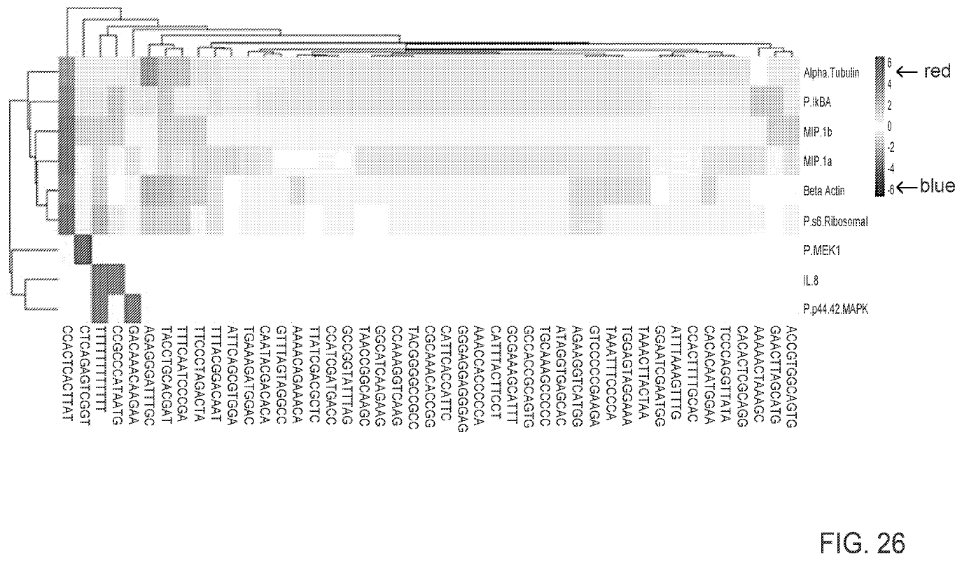

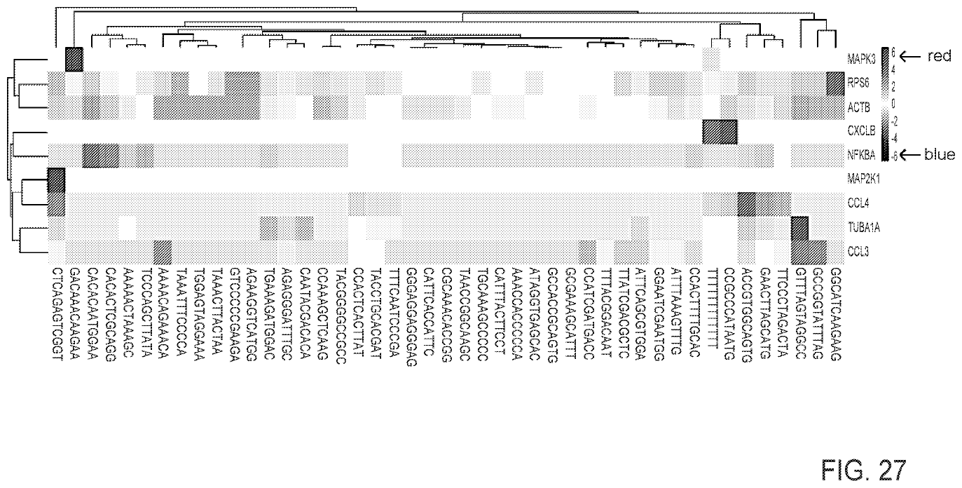

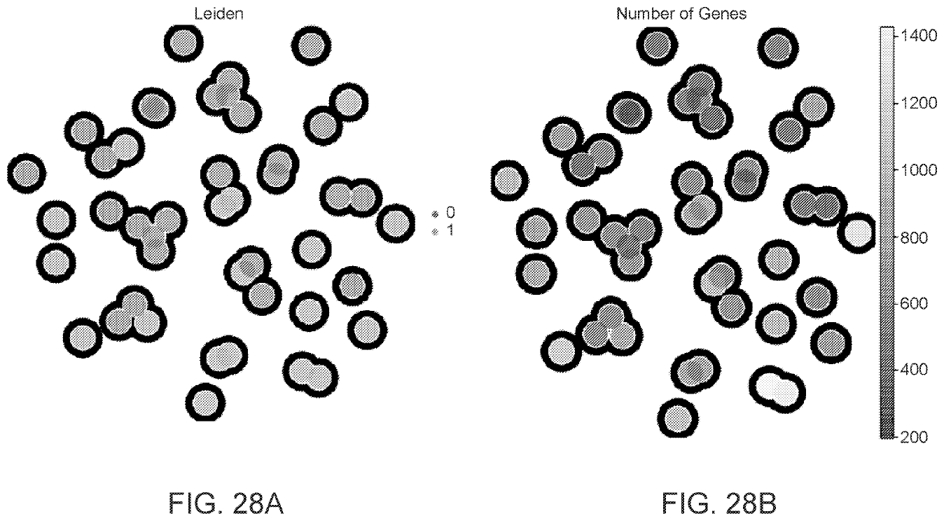

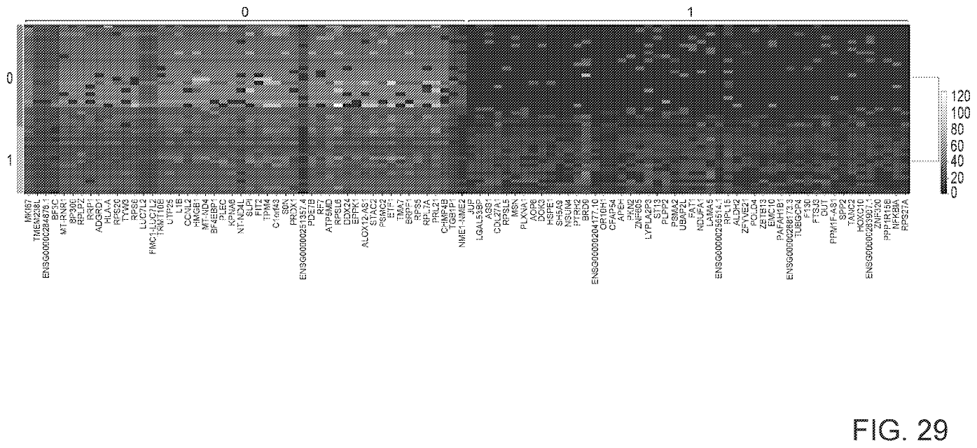

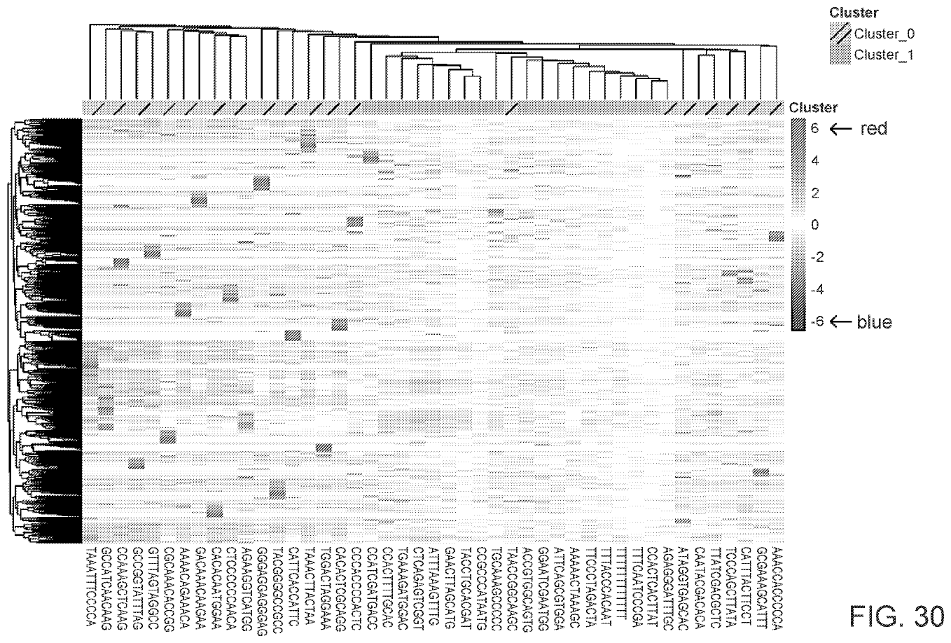

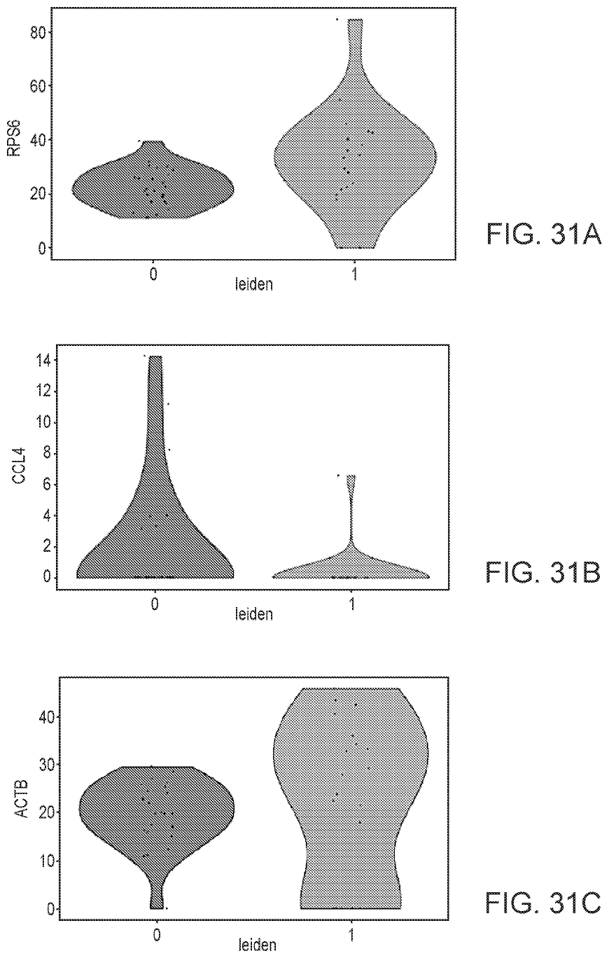

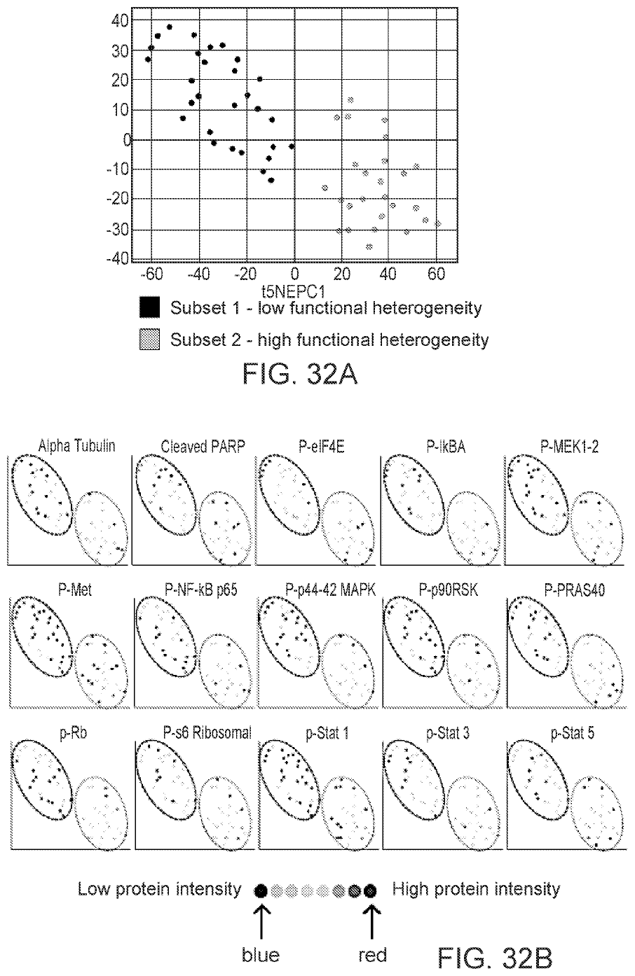

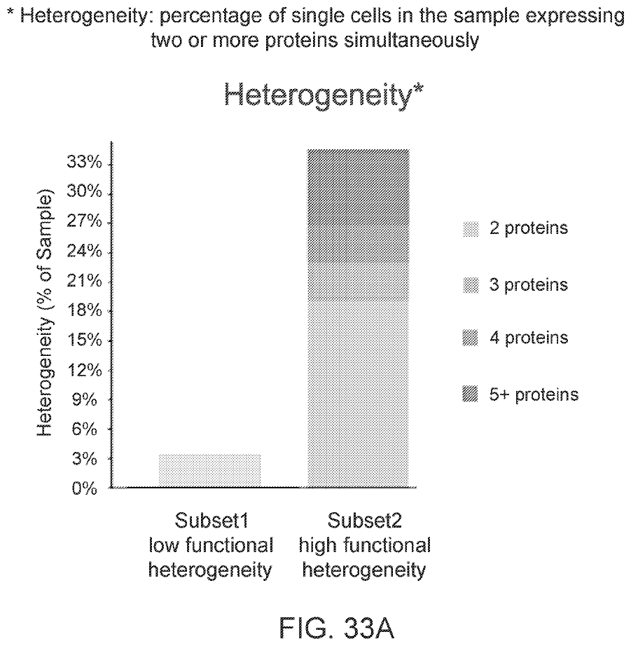

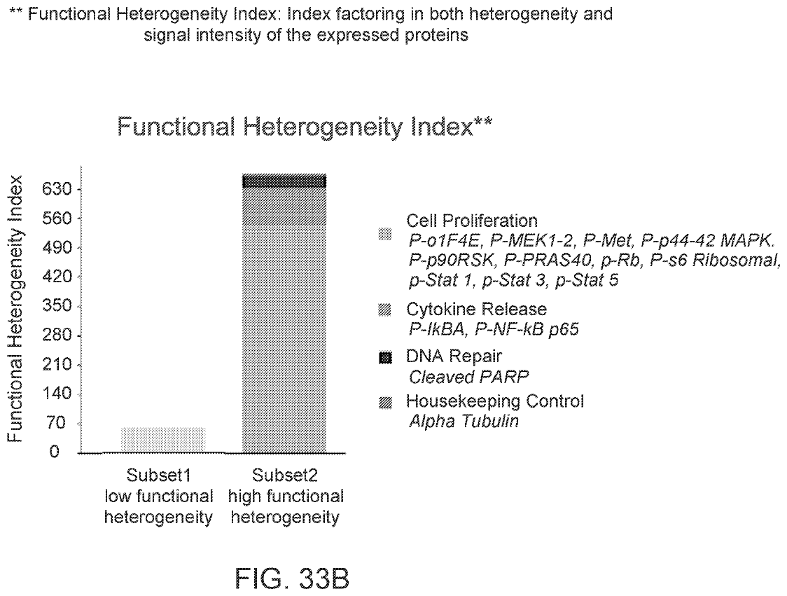

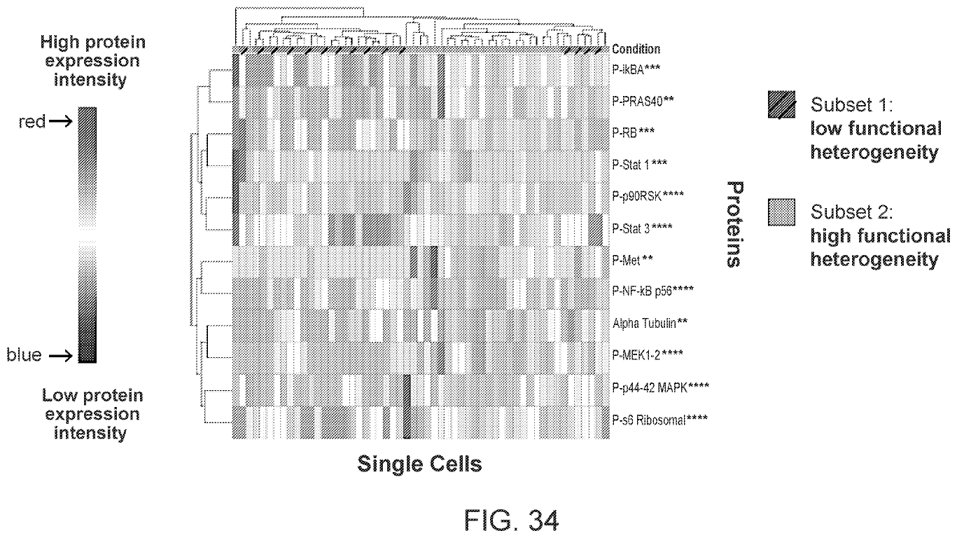

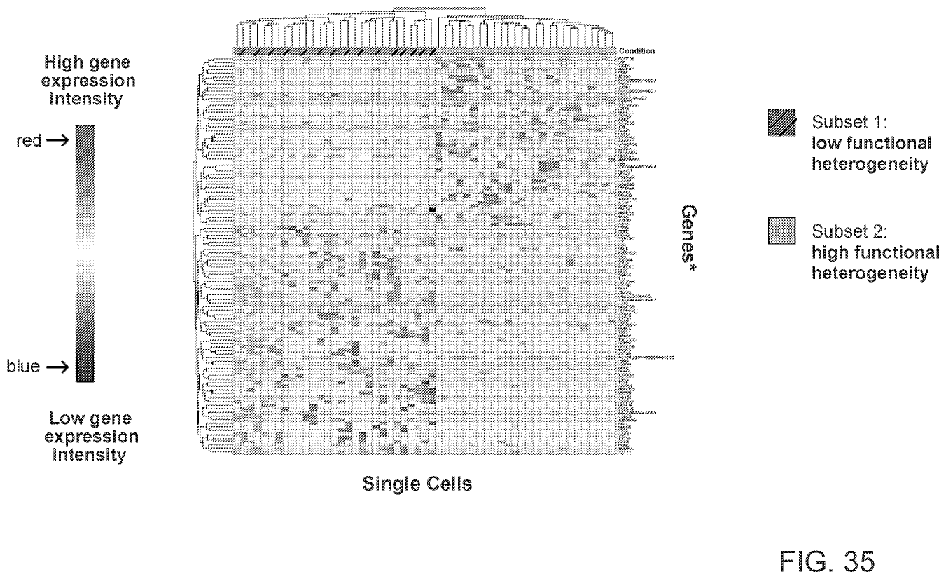

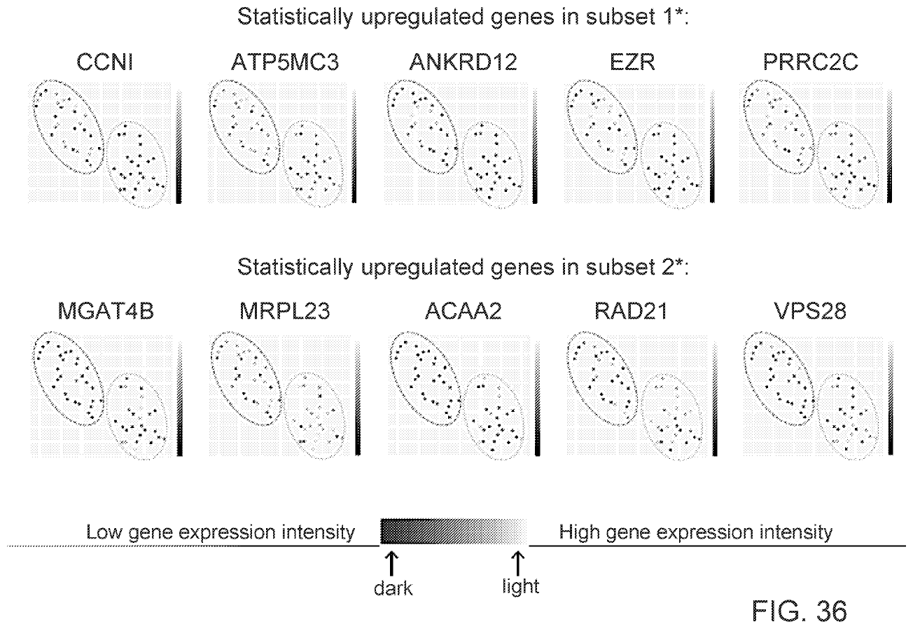

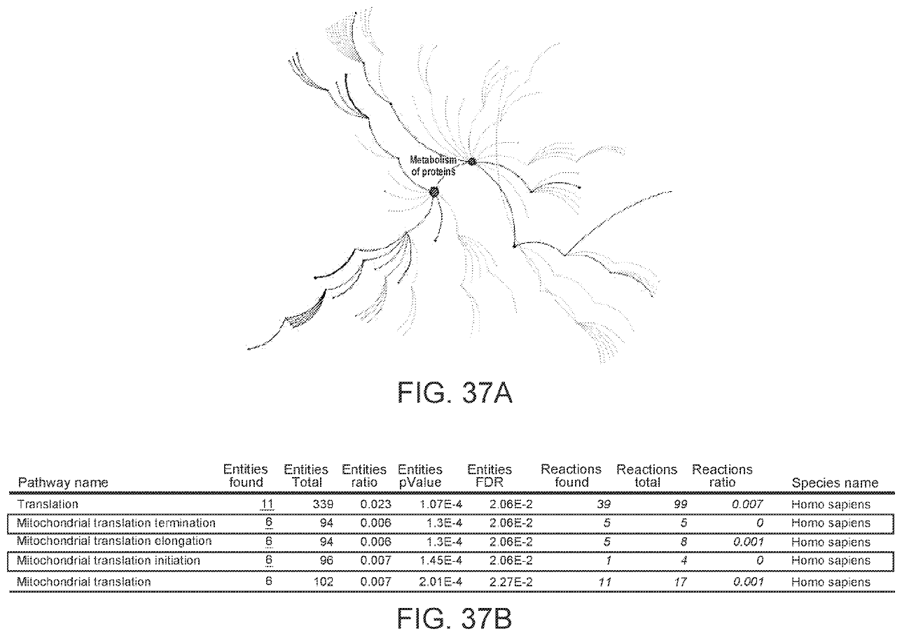

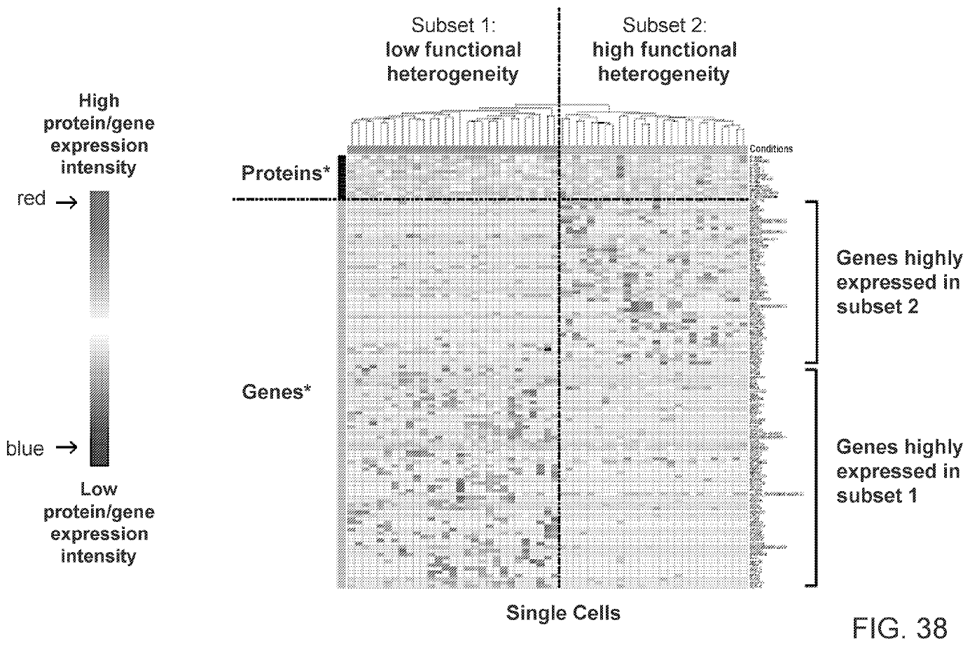

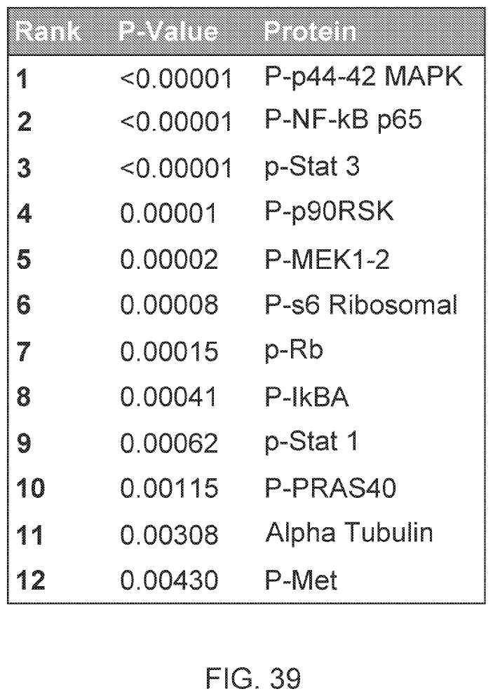

Turning now to the Drawings, FIG. 1 is an illustration of a device configured for simultaneous genomic, transcriptomic, and proteomic analysis of single cells according to compositions and methods of the disclosure.

In some embodiments, as shown in FIG. 2A, a method to manufacture barcoded micro-beads (in some embodiments, barcoded DNA micro-beads), for loading into assay chip-devices, and in some embodiments, into single-cell chambers of a substrate (see, e.g., steps 1-3). FIG. 2A also illustrates steps according to other embodiments which, in some embodiments, are combined with steps 1-3, and each additional step, or a step on its own, represents a specific embodiment, e.g., see steps 4-8. In some embodiments, barcoded DNA micro-beads are capture beads (CBs). Described in FIG. 2A as a well or as a microwell, such feature may be described below as a chamber or as a chamber comprising at least one pocket, as such terms may be used interchangeably.

FIG. 2B is an additional illustration related to the method of manufacture of chip devices (see above), illustrating DNA barcoded micro-beads loaded single-cell chips, and (in some embodiments, 8-cycle barcode reading.

FIG. 2C is an additional illustration related to the method of manufacture of chip devices (see above), illustrating DNA barcoded micro-beads loaded single-cell chips, and (in some embodiments, 4-cycle barcode reading).

FIG. 2D is an illustration depicting a polynucleotide sequence of the DNA barcoded micro-beads. In some embodiments, the polynucleotide sequence comprises any one or more of a split-and-pool nucleotide for cell barcode sequence (J), a unique molecular indicator sequence (N), and a A, C, or G nucleotide (V).

FIG. 2E is an illustration depicting a DNA barcoded micro-bead. In some embodiments, the polynucleotide sequence comprises any one or more of a 5T spacer sequence, a PCR handle sequence, a Unique Molecular Identifier (UMI) sequence, a cell barcode sequence, and a sequencing handle sequence. In some embodiments, a capture sequence is hybridized to the sequencing handle via priming sequence. In some embodiments, the capture sequences comprise a polyT sequence for mRNA capture. In some embodiments, the capture sequences comprise a sequence-specific or gene-specific capture sequence.

In some embodiments, as shown in FIG. 3A, is a method to perform valve-less lysis of cells on assay chip-devices in single-cell chambers of a substrate each comprising a DNA barcoded micro-bead. In some embodiments, the substrate is unclamped thereby allowing for cell lysis buffer to flow from an inlet port to an outlet port between the substrate and glass slide. In some embodiments, following addition of lysis buffer, the substrate and glass slide are clamped together to isolate each single-cell chambers of the substrate each comprising a DNA barcoded micro-bead with lysis buffer to lyse the single cell.

FIG. 3B is an illustration depicting the assay chip-devices of the disclosure configured for valve-less cell lysis. In some embodiments, the central portion of the device is initially clamped thereby constraining cell lysis buffer to flow from the inlet port to the outlet port via a perimeter portion. The perimeter portion can be filled with a predetermined volume of cell lysis buffer. In some embodiments, the central portion of the device is unclamped and cell lysis buffer is allowed to enter the central portion of the device from the perimeter portion of the device in order to lyse cells, as described and depicted in FIG. 3A.

FIG. 4 is an illustration depicting assay chip-devices of the disclosure configured for valve-less cell lysis. In some embodiments, the barcoded micro-beads (in some embodiments, barcoded DNA micro-beads), for loading into assay chip-devices, and in some embodiments, into single-cell chambers of a substrate. In some embodiments, the surface comprises a plurality of substantially parallel lines of capture antibodies for detecting cellular proteins and metabolites. In some embodiments, the surface comprises a fiducial line for aligning the surface and the substrate.

FIG. 5 is an illustration depicting assay chip-devices of the disclosure. In some embodiments, the substrate comprises a plurality of single-cell chambers each configured to retain a single barcoded DNA micro-beads.

As shown in the dashed, magnified image of FIG. 5, the assay chip-devices of the present disclosure include at least one chamber, or micro-chamber, as well as at least one pocket therein. FIG. 5 illustrates a plurality of chambers, each having a pocket that is center-aligned with a respective longitudinal axis of the chamber. Compared between adjacent chambers, however, the pockets are not aligned but are alternating. Remaining central within the chamber, the alternating position of the pockets improves, among other things, image resolution quality.

In some embodiments, the single cell chambers comprise a pocket for retaining the DNA micro-bead wherein the pocket arranged therein is in a position offset from a center of the chamber and having a pocket diameter greater than the width of the chamber. In some embodiments, the barcoded DNA micro-beads capture nucleic acids from lysed single cells. In some embodiments, the captured nucleic acids are processed and converted to cDNA.

FIG. 6A is an illustration depicting assay chip-devices of the disclosure configured for valve-less cell lysis. In some embodiments, the barcoded micro-beads (in some embodiments, barcoded DNA micro-beads), for loading into assay chip-devices, and in some embodiments, into single-cell chambers of a substrate. In some embodiments, the surface comprises a plurality of substantially parallel lines of capture antibodies for detecting cellular proteins and metabolites. In some embodiments, the surface comprises a fiducial line for aligning the surface and the substrate.

FIG. 6B is an illustration depicting capture of an mRNA with a barcoded DNA micro-bead (i.e. Capture bead (CB)) by hybridizing the polyA sequence of the mRNA to the polyT sequence of the barcoded DNA micro-bead. In some embodiments, the mRNA is captured from cell lysate. In some embodiments, a captured mRNA is converted to cDNA using reverse transcription. In some embodiments, the cDNA incorporates the barcode sequence, UMI sequence, cell barcode sequence, handle sequence, template switch oligonucleotide (TSO) handle and captured mRNA into a single sequence.

FIG. 6C is an illustration depicting capture of an mRNA with a barcoded DNA micro-bead by hybridizing the polyA sequence of the mRNA to the polyT sequence of the barcoded DNA micro-bead. In some embodiments, the mRNA is captured from cell lysate. I some embodiments, a captured mRNA is converted to cDNA using reverse transcription. In some embodiments, the cDNA incorporates the barcode sequence, UMI sequence, cell barcode sequence, handle sequence, TSO handle and captured mRNA into a single sequence.

FIG. 6D is an illustration depicting nucleic acid library preparation from cDNA. In some embodiments, a PCR handle sequence and TSO handle sequence are used to generate the nucleic acid library. In some embodiments, the gene transcripts and cell barcodes permit spatial determination of where each cell was located on the substrate of the assay chip-device.

FIG. 7A is an illustration depicting assay chip-devices of the disclosure. In some embodiments, the substrate comprises a plurality of single-cell chambers each configured to retain a single barcoded DNA micro-beads. In some embodiments, the single cell chambers comprise a pocket for retaining the DNA micro-bead configured to capture sequence-specific nucleic acids wherein the pocket arranged therein in a position offset from a center of the chamber and having a pocket diameter greater than the width of the chamber. In some embodiments, the barcoded DNA micro-beads capture targeted nucleic acids from lysed single cells. In some embodiments, the captured nucleic acids are processed and converted to cDNA.

FIG. 7B is an illustration depicting a DNA barcoded micro-bead (i.e. capture bead). In some embodiments, the polynucleotide sequence comprises any one or more of a 5T spacer sequence, a PCR handle sequence, a UMI sequence, a cell barcode sequence, and a barcode handle sequence. In some embodiments, a capture sequence is hybridized to the barcode handle via priming sequence. In some embodiments, the capture sequences comprise a sequence-specific or gene-specific capture sequence. In some embodiments, the hybridized capture sequence, or complement thereof, is extended onto the barcode handle via polymerase to generate the capture bead. In some embodiments, the barcode handle sequence is used to hybridize and thus extend the capture sequence onto the capture bead before beads are loaded onto the chip (i.e. into the chamber). In some embodiments, the barcode handle sequence is used to hybridize and thus extend the capture sequence onto the capture bead after beads are loaded onto the chip (i.e. into the chamber).

### Devices and Arrays

Devices and arrays are provided and configured for use in methods of analyzing multiple cellular activities and pathways in single cells in a high-throughput format wherein hundreds of intracellular components, including DNA, RNA, and protein levels of each single cell of a plurality of thousands of cells are analyzed in parallel.

FIG. 8 is a schematic depiction of a multiplex assay chip device, according to an embodiment. The multiplex assay chip device can be used for multiplexed analysis of biological material. As shown, the multiplex assay chip device includes a chamber C coupled to a first pocket P(1) and a second pocket P(2). In some embodiments, the chamber C can include an open end on a first side of the multiplex assay chip device. In some embodiments, the open end of the chamber C can be physically coupled to a central atrium. In some embodiments, a surface can cover the open end of the chamber C, fluidically isolating the chamber C from the rest of the multiplex assay chip device. In some embodiments, the first pocket P(1) and the second pocket P(2) can be connected and/or arranged along the chamber. Stated another way, the chamber comprises the pocket P(1) and pocket P(2). In some embodiments, the chamber C and the pockets of the multiplex assay chip device can be included in a substrate. In some embodiments, the substrate can be at least partially composed of a polymer. In some embodiments, the polymer can include polydimethylsiloxane (PDMS).

In some embodiments, the multiplex assay chip device of the disclosure includes a chamber C coupled to a first pocket P(1). the open end of the chamber C can be physically coupled to a central atrium. In some embodiments, a surface can cover the open end of the chamber C, fluidically isolating the chamber C from the rest of the multiplex assay chip device. In some embodiments, the first pocket P(1) can be connected and/or arranged along the chamber. Stated another way, the chamber comprises the pocket P(1). In some embodiments, the chamber C and the pocket of the multiplex assay chip device can be included in a substrate. In some embodiments, the substrate can be at least partially composed of a polymer. In some embodiments, the polymer can include polydimethylsiloxane (PDMS).

As shown, the multiplex assay chip device includes one chamber (C). In some embodiments, the multiplex assay chip device can include at least about one chamber, at least about 2 chambers, at least about 3 chambers, at least about 4 chambers, at least about 5 chambers, at least about 6 chambers, at least about 7 chambers, at least about 8 chambers, at least about 9 chambers, at least about 10 chambers, at least about 20 chambers, at least about chambers, at least about 40 chambers, at least about 50 chambers, at least about 60 chambers, at least about 70 chambers, at least about 80 chambers, at least about 90 chambers, at least about 100 chambers, at least about 200 chambers, at least about 300 chambers, at least about 400 chambers, at least about 500 chambers, at least about 600 chambers, at least about 700 chambers, at least about 800 chambers, at least about 900 chambers, at least about 1,000 chambers, at least about 2,000 chambers, at least about 2,500 chambers, at least about 3,000 chambers, at least about 4,000 chambers, at least about 5,000 chambers, at least about 6,000 chambers, at least about 7,000 chambers, at least about 8,000 chambers, at least about 9,000 chambers, at least about 10,000 chambers, at least about 11,000 chambers, at least about 12,000 chambers, at least about 13,000 chambers, at least about 14,000 chambers, at least about 15,000 chambers, at least about 16,000 chambers, at least about 17,000 chambers, at least about 18,000 chambers, at least about 19,000 chambers, at least about 20,000 chambers, at least about chambers, at least about 100,000 chambers, or at least about 200,000 chambers. In some embodiments, the multiplex assay chip device can include no more than about 200,000 chambers, no more than about 100,000 chambers, no more than about 50,000 chambers, no more than about 20,000 chambers, no more than about 19,000 chambers, no more than about 18,000 chambers, no more than about 17,000 chambers, no more than about 16,000 chambers, no more than about 15,000 chambers, no more than about 14,000 chambers, no more than about 13,000 chambers, no more than about 12,000 chambers, no more than about 11,000 chambers, no more than about 10,000 chambers, no more than about 9,000 chambers, no more than about 8,000 chambers, no more than about 7,000 chambers, no more than about 6,000 chambers, no more than about 5,000 chambers, no more than about 4,000 chambers, no more than about 3,000 chambers, no more than about 2,500 chambers, no more than about 2,000 chambers, no more than about 1,000 chambers, no more than about 900 chambers, no more than about 800 chambers, no more than about 700 chambers, no more than about 600 chambers, no more than about 500 chambers, no more than about 400 chambers, no more than about 300 chambers, no more than about 200 chambers, no more than about 100 chambers, no more than about 90 chambers, no more than about 80 chambers, no more than about 70 chambers, no more than about 60 chambers, no more than about 50 chambers, no more than about 40 chambers, no more than about 30 chambers, no more than about 20 chambers, no more than about 10 chambers, no more than about 9 chambers, no more than about 8 chambers, no more than about 7 chambers, no more than about 6 chambers, no more than about 5 chambers, no more than about 4 chambers, or no more than about 3 chambers.

Combinations of the above-referenced number of chambers in the multiplex assay chip device are also possible (e.g., at least about 2 chambers and no more than about 200,000 chambers or at least about 2 chambers and no more than about 2,500 chambers), inclusive of all values and ranges therebetween. In some embodiments, the multiplex assay chip device can include about 2 chambers, about 3 chambers, about 4 chambers, about 5 chambers, about 6 chambers, about 7 chambers, about 8 chambers, about 9 chambers, about 10 chambers, about chambers, about 30 chambers, about 40 chambers, about 50 chambers, about 60 chambers, about 70 chambers, about 80 chambers, about 90 chambers, about 100 chambers, about 200 chambers, about 300 chambers, about 400 chambers, about 500 chambers, about 600 chambers, about 700 chambers, about 800 chambers, about 900 chambers, about 1,000 chambers, about 2,000 chambers, about 2,500 chambers, about 3,000 chambers, about 4,000 chambers, about chambers, about 6,000 chambers, about 7,000 chambers, about 8,000 chambers, about 9,000 chambers, about 10,000 chambers, about 11,000 chambers, about 12,000 chambers, about 13,000 chambers, about 14,000 chambers, about 15,000 chambers, about 16,000 chambers, about 17,000 chambers, about 18,000 chambers, about 19,000 chambers, about chambers, about 50,000 chambers, about 100,000 chambers, or about 200,000 chambers.

In some embodiments, the chamber C can be composed of glass, polymers, metal, silica, or any other suitable material. In some embodiments, the chamber C can have a circular cross section, a rectangular cross section, a square cross section, a pentagonal cross section, an elliptical cross section, a hexagonal cross section, or any other suitable cross-sectional shape. In some embodiments, each of the chamber C can include an open end on a first side of the multiplex assay chip device. In some embodiments, each of the open ends of the chamber C can be fluidically coupled to a central atrium. In some embodiments of systems with multiple chambers, a surface can cover the open ends of either of the chambers, fluidically isolating the chambers from one another.

In some embodiments, the chamber C can have a length of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, or at least about 1,000 μm, at least about 1,500 μm, at least about 1,800 μm, at least about 1,860 μm or at least about 2000 μm. In some embodiments, the chamber C can have a length of no more than about 2,000 μm, no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced lengths of the chamber C are also possible (e.g., at least about 1 μm and no more than about 2,000 μm or at least about 5 μm and no more than about 200 μm) inclusive of all values and ranges therebetween. In some embodiments, the chamber C can have a length of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1,000 μm, about 1,500 μm, about 1,800 μm, about 1,860 μm, or about 2,000 μm.

In some embodiments of systems with multiple chambers, the chambers can have uniform lengths. In other words, a first chamber can have a length the same or substantially similar to a second chamber. In some embodiments, the chambers can have different lengths. In other words, a first chamber can have a length different from the length of a second chamber.

In some embodiments, the chamber C can have a width (L) of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 25 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, or at least about 400 μm. In some embodiments, the chamber C can have a width of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 25 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced widths of the chamber C are also possible (e.g., at least about 1 μm and no more than about 500 μm or at least about 5 μm and no more than about 200 μm) inclusive of all values and ranges therebetween. In some embodiments, the chamber C can have a width of about 1 μm, about 2 μm, about 3 μm, about 4 μm, about 5 μm, about 6 μm, about 7 μm, about 8 μm, about 9 μm, about 10 μm, about 20 μm, about 25 μm, about 30 μm, about 40 μm, about 50 μm, about 60 μm, about 70 μm, about 80 μm, about 90 μm, about 100 μm, about 200 μm, about 300 μm, about 400 μm, or about 500 μm.

In some embodiments of systems with multiple chambers, the chambers can have uniform widths. In other words, a first chamber can have a width the same or substantially similar to a second chamber. In some embodiments, the chambers can have different lengths. In other words, a first chamber can have a width different from the width of a second chamber.

In some embodiments, the chamber C can have a depth of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 55 μm, at least about 60 μm, at least about 70 μm, at least about 80 μm, at least about 90 μm, at least about 100 μm, at least about 200 μm, at least about 300 μm, or at least about 400 μm. In some embodiments, the chamber C can have a depth of no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 55 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced depths of the chamber C are also possible (e.g., at least about 1 μm and no more than about 500 μm or at least about 5 μm and no more than about 200 μm) inclusive of all values and ranges therebetween. In some embodiments, the chamber C can have a depth of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 55 μm, about 60 μm, about 70 μm, about 80 μm, about 90 μm, about 100 μm, about 200 μm, about 300 μm, about 400 μm, or about 500 μm.

In some embodiments of systems with multiple chambers, the chambers can have uniform depths. In other words, a first chamber can have a depth the same or substantially similar to a second chamber. In some embodiments, the chambers can have different lengths. In other words, a first chamber can have a depth different from the depth of a second chamber.

In some embodiments, a surface (not shown) can couple with the chamber C to cover the chamber C. In some embodiments of systems with multiple chambers, the surface can fluidically isolate chambers from one another. For example, the surface can isolate a first chamber from a second chamber. In some embodiments, the surface can include glass. In some embodiments, the surface can include a plurality of substantially parallel lines of capture antibodies. In some embodiments, each line of capture antibodies can include a different antibody, relative to antibodies of adjacent lines of capture antibodies. In some embodiments, each antibody can be configured to bind to a different target molecule. In some embodiments, at least a portion of each of the plurality of substantially parallel lines are arranged to be exposed to each chamber.

In some embodiments, the plurality of substantially parallel lines of capture antibodies can include at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, or at least about 190 different capture antibodies. In some embodiments, the plurality of substantially parallel lines of capture antibodies can include no more than about 200, no more than about 190, no more than about 180, no more than about 170, no more than about 160, no more than about 150, no more than about 140, no more than about 130, no more than about 120, no more than about 110, no more than about 100, no more than about 90, no more than about 80, no more than about 70, no more than about 60, no more than about 50, no more than about 40, no more than about 30, no more than about 20, no more than about 10, no more than about 9, no more than about 8, no more than about 7, no more than about 6, no more than about 5, no more than about 4, or no more than about 3 different capture antibodies. Combinations of the above-referenced numbers of different capture antibodies are also possible (e.g., at least about 2 and no more than about 200 or at least about 2 and no more than about 50), inclusive of all values and ranges therebetween. In some embodiments, the plurality of substantially parallel lines of capture antibodies can include about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 50, about 60, about 70, about 80, about 90, about 100, about 110, about 120, about 130, about 140, about 150, about 160, about 170, about 180, about 190, or about 200 different capture antibodies. In some embodiments, each chamber of the plurality of chambers is configured to contact the plurality of plurality of substantially parallel lines of capture antibodies at least once. In some embodiments, each chamber of the plurality of chambers is configured to contact the plurality of plurality of substantially parallel lines of capture antibodies at least twice (i.e. each capture antibody of the plurality of substantially parallel lines of capture antibodies contacts the chamber at least twice).

In some embodiments, each substantially parallel line of capture antibodies comprises a single species of antibody specific for a single biological target molecule. In some embodiments, each substantially parallel line of capture antibodies can comprise at least two species of antibody, each species specific for a different biological target molecule. In some embodiments, each substantially parallel line of capture antibodies can comprise at least three species of antibody, each species specific for a different biological target molecule. In some embodiments, each substantially parallel line of capture antibodies can comprise at least four species of antibody, each species specific for a different biological target molecule. In cases where a substantially parallel line of capture antibodies comprises two or more species of capture antibodies, each species of capture antibody can be detected by a distinct secondary detection antibody each having a spectrally-distinguishable label (i.e. fluorescent label).

As shown in FIG. 8, the multiplex assay chip device includes two pockets (P(1) and P(2)). In some embodiments, the multiplex assay chip device can include at least about 1 pocket, at least about 2 pockets, at least about 3 pockets (as in FIG. 9), at least about 4 pockets, at least about 5 pockets, at least about 6 pockets, at least about 7 pockets, at least about 8 pockets, at least about 9 pockets, at least about 10 pockets, at least about 20 pockets, at least about 30 pockets, at least about 40 pockets, at least about 50 pockets, at least about 60 pockets, at least about 70 pockets, at least about 80 pockets, at least about 90 pockets, at least about 100 pockets, at least about 200 pockets, at least about 300 pockets, at least about 400 pockets, at least about 500 pockets, at least about 600 pockets, at least about 700 pockets, at least about 800 pockets, at least about 900 pockets, at least about 1,000 pockets, at least about 2,000 pockets, at least about 2,500 pockets, at least about 3,000 pockets, at least about 4,000 pockets, at least about 5,000 pockets, at least about 6,000 pockets, at least about 7,000 pockets, at least about 8,000 pockets, at least about 9,000 pockets, at least about 10,000 pockets, at least about 11,000 pockets, at least about 12,000 pockets, at least about 13,000 pockets, at least about 14,000 pockets, at least about 15,000 pockets, at least about 16,000 pockets, at least about 17,000 pockets, at least about 18,000 pockets, or at least about 19,000 pockets. In some embodiments, the multiplex assay chip device can include no more than about 20,000 pockets, no more than about 19,000 pockets, no more than about 18,000 pockets, no more than about 17,000 pockets, no more than about 16,000 pockets, no more than about 15,000 pockets, no more than about 14,000 pockets, no more than about 13,000 pockets, no more than about 12,000 pockets, no more than about 11,000 pockets, no more than about 10,000 pockets, no more than about 9,000 pockets, no more than about 8,000 pockets, no more than about 7,000 pockets, no more than about 6,000 pockets, no more than about 5,000 pockets, no more than about 4,000 pockets, no more than about 3,000 pockets, no more than about 2,500 pockets, no more than about 2,000 pockets, no more than about 1,000 pockets, no more than about 900 pockets, no more than about 800 pockets, no more than about 700 pockets, no more than about 600 pockets, no more than about 500 pockets, no more than about 400 pockets, no more than about 300 pockets, no more than about 200 pockets, no more than about 100 pockets, no more than about 90 pockets, no more than about 80 pockets, no more than about 70 pockets, no more than about 60 pockets, no more than about 50 pockets, no more than about 40 pockets, no more than about 30 pockets, no more than about 20 pockets, no more than about 10 pockets, no more than about 9 pockets, no more than about 8 pockets, no more than about 7 pockets, no more than about 6 pockets, no more than about 5 pockets, no more than about 4 pockets, no more than about 3 pockets, no more than about 2 pockets, or no more than about 1 pockets.

Combinations of the above-referenced number of pockets in the multiplex assay chip device are also possible (e.g., at least about 1 pocket and no more than about 20,000 pockets or at least about 1 pocket and no more than about 2,500 pockets), inclusive of all values and ranges therebetween. In some embodiments, the multiplex assay chip device can include about 1 pocket, about 2 pockets, about 3 pockets, about 4 pockets, about 5 pockets, about 6 pockets, about 7 pockets, about 8 pockets, about 9 pockets, about 10 pockets, about 20 pockets, about pockets, about 40 pockets, about 50 pockets, about 60 pockets, about 70 pockets, about 80 pockets, about 90 pockets, about 100 pockets, about 200 pockets, about 300 pockets, about 400 pockets, about 500 pockets, about 600 pockets, about 700 pockets, about 800 pockets, about 900 pockets, about 1,000 pockets, about 2,000 pockets, about 2,500 pockets, about 3,000 pockets, about 4,000 pockets, about 5,000 pockets, about 6,000 pockets, about 7,000 pockets, about 8,000 pockets, about 9,000 pockets, about 10,000 pockets, about 11,000 pockets, about 12,000 pockets, about 13,000 pockets, about 14,000 pockets, about 15,000 pockets, about 16,000 pockets, about 17,000 pockets, about 18,000 pockets, about 19,000 pockets, or about 20,000 pockets.

In some embodiments, the pockets can be composed of glass, polymers, metal, silica, or any other suitable material. In some embodiments, the pockets can have a circular cross section, a rectangular cross section, a square cross section, a pentagonal cross section, an elliptical cross section, a hexagonal cross section, or any other suitable cross-sectional shape.

In some embodiments, the pockets can have a diameter or cross-sectional width of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 55 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, or at least about 900 μm. In some embodiments, the pockets can have a diameter or cross-sectional width of no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 55 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced diameters or cross-sectional widths of the pockets are also possible (e.g., at least about 1 μm and no more than about 1,000 μm or at least about 5 μm and no more than about 200 μm) inclusive of all values and ranges therebetween. In some embodiments, the pockets can have a diameter or cross-sectional width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 55 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, or about 1,000 μm.

In some embodiments, the pockets can have uniform diameters or cross-sectional widths. In other words, the first pocket P(1) can have a diameter the same or substantially similar to the second pocket P(2). In some embodiments, the pockets can have different lengths. In other words, the first pocket P(1) can have a diameter different from the diameter of the second pocket P(2).

In some embodiments, the difference in diameter between pockets of the multiplex assay chip device can be at least about 5 μm, at least about 10 μm, at least about 15 μm, at least about 20 μm, at least about 25 μm, at least about 30 μm, at least about 35 μm, at least about 40 μm, or at least about 45 μm. In some embodiments, the difference in diameter between pockets of the multiplex assay chip device can be no more than about 50 μm, no more than about 45 μm, no more than about 40 μm, no more than about 35 μm, no more than about 30 μm, no more than about 25 μm, no more than about 20 μm, no more than about 15 μm, or no more than about 10 μm. Combinations of the above-referenced differences in diameter between the pockets of the multiplex assay chip device are also possible (e.g., at least about 5 μm and no more than about 50 μm or at least about 20 μm and no more than about 40 μm), inclusive of all values and ranges therebetween. In some embodiments, the difference in diameter between pockets of the multiplex assay chip device can be about 5 μm, about 10 μm, about 15 μm, about 20 μm, about 25 μm, about 30 μm, about 35 μm, about 40 μm, about 45 μm, or about 50 μm. In some embodiments, one or more of the pockets can be center-aligned with respect to the width of an adjacent chamber. In some embodiments, one or more of the pockets can be not center-aligned with respect to the width of an adjacent chamber. In some embodiments, one or more of the pockets can be center-aligned with respect to the length of an adjacent chamber. In some embodiments, one or more of the pockets can be not center-aligned with respect to the length of an adjacent chamber. In some embodiments, one or more of the pockets can be positioned at the edge of the chamber with respect to the length of the chamber. In some embodiments, the pockets can be non-overlapping, positioned at an edge, or positioned elsewhere. Without wishing to be bound by theory, in some embodiments, having the pocket positioned toward the center of the chamber with respect to the length of the chamber is beneficial because cell contents (i.e. biological material) secreted or released from the cell does not have to diffuse as far long the length of the chamber before the contents can contact the capture bead located within the pocket.

FIG. 9 is a schematic depiction of a multiplex as assay chip device, according to an embodiment. The multiplex assay chip device can be used for multiplexed analysis of biological material. The multiplex assay chip device includes a chamber C as well as pockets P(1), P(2), and P(3). As shown, the pocket P(1) is center-aligned with the chamber C, while pockets P(2) and P(3) are not center-aligned with the chamber C. In other words, a central axis of the chamber C intersects with a central axis of the pocket P(1). A central axis of the chamber C does not intersect with a central axis of the pockets P(3) and P(2). In some aspects, a non-center aligned pocket can improve signal to noise ratio due to lower autofluorescent background further from the central axis of the chamber. In some aspects, a non-center aligned pocket can allow for increased contact between target biological molecules and capture arrays.

FIG. 10 is a schematic depiction of a multiplex assay chip device, according to an embodiment. The multiplex assay chip device can be used for multiplexed analysis of biological material. The multiplex assay chip device includes a chamber C. The multiplex assay chip device includes pockets P(1) and P(2) with capture beads CB(1) and CB(2) disposed therein.

As shown, the multiplex assay chip device includes two capture beads (CB(1) and CB(2)). In some embodiments, the multiplex assay chip device can include at least about 1 capture bead, at least about 2 capture beads, at least about 3 capture beads, at least about 4 capture beads, at least about 5 capture beads, at least about 6 capture beads, at least about 7 capture beads, at least about 8 capture beads, at least about 9 capture beads, at least about 10 capture beads, at least about 20 capture beads, at least about 30 capture beads, at least about 40 capture beads, at least about 50 capture beads, at least about 60 capture beads, at least about 70 capture beads, at least about 80 capture beads, at least about 90 capture beads, at least about 100 capture beads, at least about 200 capture beads, at least about 300 capture beads, at least about 400 capture beads, at least about 500 capture beads, at least about 600 capture beads, at least about 700 capture beads, at least about 800 capture beads, at least about 900 capture beads, at least about 1,000 capture beads, at least about 2,000 capture beads, at least about 2,500 capture beads, at least about 3,000 capture beads, at least about 4,000 capture beads, at least about 5,000 capture beads, at least about 6,000 capture beads, at least about 7,000 capture beads, at least about 8,000 capture beads, at least about 9,000 capture beads, at least about 10,000 capture beads, at least about 11,000 capture beads, at least about 12,000 capture beads, at least about 13,000 capture beads, at least about 14,000 capture beads, at least about 15,000 capture beads, at least about 16,000 capture beads, at least about 17,000 capture beads, at least about 18,000 capture beads, or at least about 19,000 capture beads. In some embodiments, the multiplex assay chip device can include no more than about 20,000 capture beads, no more than about 19,000 capture beads, no more than about 18,000 capture beads, no more than about 17,000 capture beads, no more than about 16,000 capture beads, no more than about 15,000 capture beads, no more than about 14,000 capture beads, no more than about 13,000 capture beads, no more than about 12,000 capture beads, no more than about 11,000 capture beads, no more than about 10,000 capture beads, no more than about 9,000 capture beads, no more than about 8,000 capture beads, no more than about 7,000 capture beads, no more than about 6,000 capture beads, no more than about 5,000 capture beads, no more than about 4,000 capture beads, no more than about 3,000 capture beads, no more than about 2,500 capture beads, no more than about 2,000 capture beads, no more than about 1,000 capture beads, no more than about 900 capture beads, no more than about 800 capture beads, no more than about 700 capture beads, no more than about 600 capture beads, no more than about 500 capture beads, no more than about 400 capture beads, no more than about 300 capture beads, no more than about 200 capture beads, no more than about 100 capture beads, no more than about 90 capture beads, no more than about 80 capture beads, no more than about 70 capture beads, no more than about 60 capture beads, no more than about 50 capture beads, no more than about 40 capture beads, no more than about 30 capture beads, no more than about 20 capture beads, no more than about 10 capture beads, no more than about 9 capture beads, no more than about 8 capture beads, no more than about 7 capture beads, no more than about 6 capture beads, no more than about 5 capture beads, no more than about 4 capture beads, no more than about 3 capture beads, no more than 2 capture beads, or no more than 1 capture bead.

Combinations of the above-referenced number of capture beads in the multiplex assay chip device are also possible (e.g., at least about 1 capture beads and no more than about 20,000 capture beads or at least about 1 capture beads and no more than about 2,500 capture beads), inclusive of all values and ranges therebetween. In some embodiments, the multiplex assay chip device can include about 1 capture bead, about 2 capture beads, about 3 capture beads, about 4 capture beads, about 5 capture beads, about 6 capture beads, about 7 capture beads, about 8 capture beads, about 9 capture beads, about 10 capture beads, about 20 capture beads, about 30 capture beads, about 40 capture beads, about 50 capture beads, about 60 capture beads, about 70 capture beads, about 80 capture beads, about 90 capture beads, about 100 capture beads, about 200 capture beads, about 300 capture beads, about 400 capture beads, about 500 capture beads, about 600 capture beads, about 700 capture beads, about 800 capture beads, about 900 capture beads, about 1,000 capture beads, about 2,000 capture beads, about 2,500 capture beads, about 3,000 capture beads, about 4,000 capture beads, about 5,000 capture beads, about 6,000 capture beads, about 7,000 capture beads, about 8,000 capture beads, about 9,000 capture beads, about 10,000 capture beads, about 11,000 capture beads, about 12,000 capture beads, about 13,000 capture beads, about 14,000 capture beads, about 15,000 capture beads, about 16,000 capture beads, about 17,000 capture beads, about 18,000 capture beads, about 19,000 capture beads, or about 20,000 capture beads.

In some embodiments, the capture beads can have a diameter or cross-sectional width of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, or at least about 900 μm. In some embodiments, the capture beads can have a diameter or cross-sectional width of no more than about 1,000 μm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm.

Combinations of the above-referenced diameters or cross-sectional widths of the capture beads are also possible (e.g., at least about 1 μm and no more than about 1,000 μm or at least about 5 μm and no more than about 200 μm) inclusive of all values and ranges therebetween. In some embodiments, the capture beads can have a diameter or cross-sectional width of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, or about 1,000 μm.

In some embodiments, the capture beads can have smaller diameters or cross-sectional widths than the pockets, in which the capture beads are disposed. For example, the capture bead CB(1) can have a smaller diameter than the pocket (P1). In some embodiments, a capture bead can have a smaller diameter or cross-sectional width than a pocket, in which the capture bead is disposed by at least about 5 μm, at least about 10 μm, at least about 15 μm, at least about 20 μm, at least about 25 μm, at least about 30 μm, at least about 35 μm, at least about 40 μm, at least about 45 μm. In some embodiments, a capture bead can have a smaller diameter or cross-sectional width than a pocket, in which the capture bead is disposed by no more than about 50 μm, no more than about 45 μm, no more than about 40 μm, no more than about 35 μm, no more than about 30 μm, no more than about 25 μm, no more than about 20 μm, no more than about 15 μm, or no more than about 10 μm. Combinations of the above-referenced differences between the diameters or cross sectional widths of a pocket and a capture bead disposed in the pocket are also possible (e.g., at least about 5 μm, and no more than about 50 μm or at least about 20 μm and no more than about 40 μm), inclusive of all values and ranges therebetween. In some embodiments, a capture bead can have a smaller diameter or cross-sectional width than a pocket, in which the capture bead is disposed by about 5 μm, about 10 μm, about 15 μm, about 20 μm, about 25 μm, about 30 μm, about 35 μm, about 40 μm, about 45 μm, or about 50 μm.

In some embodiments, the capture beads can have larger diameters or cross-sectional widths than their adjacent chambers. For example, the capture bead CB(1) can have a larger diameter than the chamber C. In some embodiments, a capture bead can have a larger diameter or cross-sectional width than its adjacent chamber by at least about 5 μm, at least about 10 μm, at least about 15 μm, at least about 20 μm, at least about 25 μm, at least about 30 μm, at least about 35 μm, at least about 40 μm, at least about 45 μm. In some embodiments, a capture bead can have a larger diameter or cross-sectional width than an adjacent chamber by no more than about 50 μm, no more than about 45 μm, no more than about 40 μm, no more than about 35 μm, no more than about 30 μm, no more than about 25 μm, no more than about 20 μm, no more than about 15 μm, or no more than about 10 μm. Combinations of the above-referenced differences between the diameters or cross sectional widths of a capture bead and an adjacent chamber are also possible (e.g., at least about 5 μm, and no more than about 50 μm or at least about 20 μm and no more than about 40 μm), inclusive of all values and ranges therebetween. In some embodiments, a capture bead can have a larger diameter or cross-sectional width than an adjacent chamber by about 5 μm, about 10 μm, about 15 μm, about 20 μm, about 25 μm, about 30 μm, about 35 μm, about 40 μm, about 45 μm, or about 50 μm.

In some embodiments, the capture beads can all have substantially the same size. In some embodiments, the capture beads can be of different sizes. For example, the capture bead CB(1) can be larger than capture bead CB(2), such that the capture bead CB(1) cannot fit in the pocket P(2). As an additional example capture CB(1) can have a diameter or cross-sectional width smaller than a diameter or cross-sectional width of pocket P(1), yet larger than a diameter or cross sectional width of pocket P(2), such that capture bead CB(1) can only fit into pocket P(1).

In some embodiments, the capture beads (e.g., CB(1), CB(2)) can include a capture moiety. In some embodiments, the capture moiety can capture nucleic acid sequences, peptides, proteins, metabolites, original molecules, or any combination thereof. In some embodiments, the capture moiety can capture DNA, RNA, or a combination thereof. In some embodiments, the DNA can include autosomal DNA, chromosomal DNA, cDNA, exosome DNA, single stranded DNA, double stranded DNA, or any combination thereof. In some embodiments, the RNA can include mRNA, rRNA, tRNA, snRNA, regulatory RNA, double stranded RNA, microRNA, exosome RNA, or any combination thereof. In some embodiments, the RNA can include a guide RNA from a CRISPR-Cas system. In some embodiments, the capture beads can include an oligonucleotide capture bead comprising a nucleic acid capture sequence tethered to a bead.

In some embodiments, the nucleic acid capturing bead can include at least about 1, at least about 2, at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1,000, at least about 10,000, at least about 100,000, at least about 1,000,000, or at least about 10,000,000 capture nucleic acid sequences. In some embodiments, the nucleic acid capturing bead can include no more than about 10,000,000, no more than about 1,000,000, no more than about 100,000, no more than about 10,000, no more than about 1,000, no more than about 900, no more than about 800, no more than about 700, no more than about 600, no more than about 500, no more than about 400, no more than about 300, no more than about 200, no more than about 100, no more than about 90, no more than about 80, no more than about 70, no more than about 60, no more than about 50, no more than about 40, no more than about 30, no more than about 20, no more than about 10, no more than about 9, no more than about 8, no more than about 7, no more than about 6, no more than about 5, no more than about 4, no more than about 3, or no more than about 2 capture nucleic acid sequences.

Combinations of the above-referenced numbers of capture nucleic acid sequences are also possible (e.g., at least about 1 and no more than about 10,000,000 capture nucleic acid sequences or at least about 100 and no more than about 500 capture nucleic acid sequences), inclusive of all values and ranges therebetween. In some embodiments, the nucleic acid capturing bead can include about 1, about 2, about 3, about 4, about 5, about 6, about 7, about 8, about 9, about 10, about 20, about 30, about 40, about 50, about 60, about 70, about 80, about 90, about 100, about 200, about 300, about 400, about 500, about 600, about 700, about 800, about 900, about 1,000, about 10,000, about 100,000, about 1,000,000, or about capture nucleic acid sequences.

In some embodiments, the capture nucleic acid sequence can include an individually unique cell barcode sequence, a PCR handle, a unique molecular identifier (UMI), a barcode handle sequence, and a capture sequence. In some embodiments, the capture nucleic acid sequence can include an individually unique cell barcode sequence, a PCR handle, a unique molecular identifier (UMI), a template switch oligonucleotide (TSO) sequence, a barcode handle sequence, and a capture sequence. In some aspects, the cell barcode sequence can comprise any number of nucleotides. In some aspects, the cell barcode sequence comprises at least 2, at least 3, at least 4, at least 5, at least 6, at least, 7 at least, 8 at least 9, at least 10, at least 11, at least 12, at least 13, at least 14, at least 15, at least 20, at least 25, at least 20, or at least 50 nucleotides. In some aspects, the UMI sequence can comprise any number of nucleotides. In some aspects, the UMI sequence comprises at least 2, at least 3, at least 4, at least 5, at least 6, at least, 7 at least, 8 at least 9, at least 10, at least 11, at least 12, at least 13, at least 14, at least 15, at least 20, at least 25, at least 20, or at least 50 nucleotides. In some embodiments, the capture sequence can include a polyT sequence for mRNA polyA capture. In some embodiments, the capture sequence can include an rGrGrG capture sequence for mRNA capture, wherein rGrGrG denotes three riboguanosines. In some embodiments, the capture sequence can include a gene-specific or sequence-specific capture sequence. In some embodiments, each nucleic acid sequence of the nucleic acid capturing bead can include a unique UMI. In some embodiments, the cell barcode sequence of the capture bead can be unique to each capture bead. In some aspects, each CB of the plurality of CBs on the chip can comprise a unique cell barcode sequence. In some embodiments, the cell barcode sequence of the capture bead can be unique to each chamber. Each CB having a unique cell barcode sequence allows captured nucleic acids to be correlated to individual single cells located inside each single cell chamber. In some aspects, each nucleic acid sequence of the nucleic acid capturing bead the sequence of cell barcode is the same for each nucleic acid of the capture bead while the UMI is unique for each sequence of the capture bead. This allows each sequence captured in methods of the disclosure to be correlated back to the origin cell while still enabling quantification of each sequence captured from a single cell in each chamber. The UMI allows for quantification of multiple copies of a captured nucleic acid that may originate from the same single cell. (i.e. multiple copies of the same mRNA transcript originating from a single gene).

In some embodiments, the capture moiety can capture proteins. In some embodiments, the capture beads can include a capture antibody tethered to a bead. In some embodiments, the protein capturing CB comprises an antibody, an aptamer, a functional fragment of an antibody, or an antibody mimetic. In some embodiments, the capture beads can include plastic, polymer, metal, silica, or any other suitable material, or combinations thereof.

In some embodiments, the capture beads can include a porous capture bead. In some embodiments, the porous capture bead can release one or more agents. In some embodiments, the one or more agents can include an enzyme, a catalyzer, a stimulatory agent, a therapeutic agent, or any combination thereof.

In some embodiments, the biological material can include a biological sample, a metabolite, a protein, a polypeptide, a cell, or any combination thereof. In some embodiments, the cell can be a single cell. In some embodiments, the single cell can include a healthy cell. In some embodiments, the single cell can include a tumor cell. In some embodiments, the healthy cell can include a malignant cell. In some embodiments, the single cell can include a neural cell. In some embodiments, the single cell can include a glial cell. In some embodiments, the single cell can include an immune cell. In some embodiments, the single cell can include a T-cell and/or a B-cell. In some embodiments, the single cell can include a bacterium. In some embodiments, the single cell can include a HeLa cell. In some embodiments, the single cell can include a monocyte. In some embodiments, the single cell can include a melanoma cell. In some embodiments, the single cell can include a Chinese hamster ovary (CHO) cell. In some embodiments, the single cell can include a yeast cell. In some embodiments, the single cell can include an alga.

In some embodiments, the single cell can be genetically modified. In some embodiments, the biological sample can be obtained from a subject. In some embodiments, the biological sample can include blood, cerebral spinal fluid (CSF) lymph fluid, plural effusion, urine, saliva, or any combination thereof. In some embodiments, the biological sample can include a cell culture media. In some embodiments, the biological sample can include a tissue sample or tissue biopsy. In some embodiments, the subject can be healthy. In some embodiments, the subject can have cancer. In some embodiments, the subject can have an infection. In some embodiments, the subject can have an autoimmune disorder. In some embodiments, the subject can have an inflammatory disorder. In some embodiments, the subject can have a neurological disorder. In some embodiments, the subject can have a metabolic disorder. In some embodiments, the subject can have a degenerative disorder. In some embodiments, the subject can have a genetic mutation or epigenetic modification associated with a disease or disorder.

FIG. 11 is a cross-sectional view of pockets, and various form factors thereof, according to various embodiments. As shown, pockets can have a circular cross section, a rectangular cross section, a pentagonal cross section, or a hexagonal cross section. In some embodiments, one or more pockets can have a square cross section, an elliptical cross section.

### Analysis of Polyfunctionality

The disclosure provides methods of assessing polyfunctionality in subject cells. Polyfunctionality is a measure of efficacy and potency of cells intended for cellular therapy. Highly polyfunctional cells, and cellular components derived from polyfunctional cells, are correlated with successful and potency of immune mediated cellular therapies. Thus, there is interest in identifying polyfunctional cells and identifying cellular components of those cells such as receptor sequences like T-cell receptors (TCR) and B-cell receptors (BCR) from polyfunctional T-cells and B-cells or antibodies secreted by polyfunctional B-cells, because these components may be effective and potent components of an cell therapy, in some aspects an autologous cell therapy.

In some aspects, the polyfunctionality of a subject cell is determined by calculating a polyfunctional strength index (PSI) for the cell. In some aspects, the PSI is calculated by determining the sum of all cytokine levels in a subject cell. In some aspects, a high PSI in a single cell can be understood as a cell secreting a large number of distinct cytokines. In some aspects, a high PSI in a single can be understood as a large overall level of cytokines (i.e. high concentration) in a single cell relative to both a non-stimulated or stimulated cell. In some aspects, the disclosure provides methods of quantifying the level of each cytokine secreted by a subject cell. In some aspects, the cytokine levels are measured in pg/mL or relative fluorescence units (RFU). In some aspects, cytokine levels are measured in molar concentration or total number of cytokine molecules secreted by the cell. In some aspects, the cytokine level is determined by measuring the signal intensity of a cytokine as detected utilizing devices of the disclosure.

In some aspects of the disclosure, a subject cell that secretes two or more cytokines is a polyfunctional cell. In some aspects, the polyfunctional subject cell secretes at least 2, at least 3, at least 4, at least 5, at least 6, at least 7, at least, 8, at least, 9, at least 10, at least 11, at least 12, at least 13, at least 14, at least 15, at least 20, at least 25, at least 30, at least 40, or at least different cytokines. In some aspects, the polyfunctional subject cell secretes no more than 2, no more than 3, no more than 4, no more than 5, no more than 10, no more than 15, no more than 25, no more than 50, no more than 100, or no more than 1000 different cytokines.

In some aspects, a polyfunctional cell can be a cell that has a high overall expression level of at least one cytokine. In some aspects, a polyfunctional cell can be a cell that has a high overall expression level of at least two cytokines.

The absolute and relative contributions of each cytokine in the secretome of a control and stimulated cells can be measured. For example, on a single-cell basis the Polyfunctional Strength Index (PSI) contributions of each detected cytokine in the secretome can be calculated. In some aspects, when a population of cells are detected individually, but analyzed as a population, a percentage of cells in the given population that express any one cytokine above a threshold level may be determined.

The polyfunctional strength index (PSI) is a metric that factors in the polyfunctionality of cells in a sample, and the signal intensity of the cytokines secreted by each cell. It is found by multiplying the percentage of polyfunctional cells of a sample (single cells secreting two or more cytokines), by the average signal intensity of these cytokines.

In some aspects, the units of the intensity levels of each cytokine is based on “on-chip” calibration curves (“on chip” may also be referred to as “in situ” with respect to the methods of the disclosure). These values can be converted to approximate pg/mL secretion amounts.

Methods and devices for multiplexed assessment of polyfunctionality in subject cells is described in U.S. patent application Ser. No. 16/332,627, the contents of which are incorporated by reference in their entirety.

### Identification of Expressed TCR Sequences

The disclosure provides devices and methods capable of simultaneously identifying at least one expressed gene or T-cell receptor (TCR) sequence and at least one cytokine at the single cell level. For example, in certain embodiments, the systems and methods of the disclosure can simultaneously identify at least one to at least 50 cytokines and at least one TCR sequence at the single cell level.

The disclosure provides devices and methods capable of identifying at least one expressed gene or at least one TCR sequence of a subject T-cell. In some aspects, the subject T-cell is a polyfunctional T-cell. In some aspects, a subject T-cell is lysed such that the subject T-cell releases at least one target RNA encoding for the at least one expressed gene or at least one TCR sequence. In some aspects, the method provides conditions sufficient for the target RNA to contact the CB to form an CB-target RNA complex. The complexed target RNA is then sequenced to determine the sequence of at least one expressed gene or at least one TCR sequence.

### Identification of Expressed BCR Sequences

The disclosure provides devices and methods capable of simultaneously identifying at least one expressed gene or B-cell receptor (BCR) sequence and at least one cytokine at the single cell level. For example, in certain embodiments, the systems and methods of the disclosure can simultaneously identify at least one to at least 50 cytokines and at least one TCR sequence at the single cell level.

The disclosure provides methods of identifying at least one expressed gene or at least one BCR sequence of a subject B-cell. In some aspects, the subject B-cell is a polyfunctional B-cell. In some aspects, a subject B-cell is lysed such that the subject B-cell releases at least one target RNA encoding for the at least one expressed gene or at least one BCR sequence. In some aspects, the method provides conditions sufficient for the target RNA to contact the CB to form an CB-target RNA complex. The complexed target RNA is then sequenced to determine the sequence of at least one expressed gene or at least one BCR sequence.

### Identifying Secreted Cytokines in Subject Cells

The disclosure provides methods of detecting secreted cytokines in subject cells to determine the degree of polyfunctionality of the subject cell. In some aspects, the disclosure provides methods of identifying and quantifying secreted cytokines from subject cells. In some aspects, the subject cell is a stem cell, myeloid cell, glial cell, neural cell, T-cell or a B-cell. In some aspects, the subject cell is a human cell.

### Cytokines

In some aspects, the disclosure provides methods of identifying and quantifying at least one cytokine. In some aspects, the disclosure provides methods of identifying and quantifying one or more cytokine. In some aspects, the methods of the disclosure comprise maintaining the subject cell in the chamber under conditions sufficient to permit: (1) the subject cell to secrete at least one cytokine and (2) at least one antibody of the antibody panel specific for the at least one protein to bind the at least one cytokine forming at least one antibody:cytokine complex.

In some aspects, the one or more cytokines are selected from the group consisting of effector, stimulatory, regulatory, inflammatory, or chemoattractive cytokines.

In some aspects, the at least one cytokine is an effector cytokine selected from the group consisting of Granzyme B, IFN-γ, M1P-1α, Performin, TNF-α, and TNF-β. In some aspects, the at least one cytokine is a stimulatory cytokine selected from the group consisting of GM-CSF, IL-12, IL-15, IL-2, IL-21, IL-5, IL-7, IL-8 and IL-9. In some aspects, the at least one cytokine is a chemoattractive cytokine selected from the group consisting of CCL-11, IP-10, MIP-1β and RANTES. In some aspects, the at least one cytokine is a regulatory cytokine selected from the group consisting of IL-10, IL-13, IL-22, IL-4, TGF-β1, sCD137 and sCD40L. In some aspects, the at least one cytokine is an inflammatory cytokine selected from the group consisting of IL-17A, IL-17F, IL-1β, IL-6, MCP-1 and MCP-4.

### Cell Stimulation

The disclosure provides methods of stimulating subject cells to induce cytokine secretion so that the polyfunctionality of the cell can be assessed. In some aspects, subject cells are within a heterogenous cell population. In some aspects, the subject cell within a heterogenous cell population is stimulated. In some aspects, the subject cell can be any type of subject cell. In some aspects, the subject cell can be a T-cell or a B-cell. In some aspects, stimulation induces cytokine production and secretion in the subject cell.

In some aspects, stimulation occurs prior to introducing the subject cell to at least one chamber of the plurality of chambers. In some aspects, stimulation occurs after introducing the subject cell to at least one chamber of the plurality of chambers.

In some aspects, stimulation comprises contacting the subject cell with a target cell or a stimulatory agent under conditions to permit stimulation of the subject cell. In some aspects, stimulation comprises contacting the subject cell with a stimulatory agent under conditions to permit stimulation of the subject cell.

In some aspects, the stimulatory agent comprises a stimulatory antibody. In some aspects, the stimulatory antibody is a monoclonal antibody. In some aspects, the monoclonal antibody is a fully human antibody. In some aspects, the monoclonal antibody is a humanized antibody, a chimeric antibody, a recombinant antibody or a modified antibody.

In some aspects, the modified antibody comprises one or more sequence variations when compared to a fully human version of an antibody having the same epitope specificity, one or more modified or synthetic amino acids, or a chemical moiety to enhance a stimulatory function.

In some aspects, the stimulatory antibody specifically binds an epitope of a T-cell regulator protein. In some aspects, the T-cell regulator protein comprises programmed cell death protein 1 (PD-1).

In some aspects, the stimulatory antibody comprises Nivolumab or a biosimilar thereof.

In some aspects, the stimulatory agent comprises a stimulatory ligand. In some aspects, the stimulatory ligand comprises programmed death ligand 1 (PD-L1).

In some aspects, the methods of the disclosure further comprise the step of disrupting contact between the subject T-cell and the target cell or the stimulatory agent.

In some aspects, the methods of the disclosure further comprise the step of depleting the target cell or the stimulatory agent from the composition.

### T-Cells

The disclosure provides methods of identifying TCR sequences expressed in single T-cells. In some aspects, the T-cell is a naïve T-cell, an activated T-cell, an effector T-cell, a helper T-cell, a cytotoxic T-cell, a gamma-delta T-cell, a regulatory T-cell, a memory T-cell, or a memory stem T-cell. In some aspects, the T-cell is an engineered T-cell. In some aspects, the T-cell is a gene modified T-cell. In some aspects, the gene modified T-cell is a Car-T-cell or a TCR-T-cell.

### T-Cell Receptors

The disclosure provides methods of identifying at least one T-cell receptor (TCR) sequence. In some aspects, the TCR sequence is encoded by an mRNA sequence.

In some aspects, the TCR sequence comprises a TCR-alpha chain sequence. In some aspects, the TCR sequence comprises a TCR-beta chain sequence. In some aspects, the TCR sequence comprises a CD3-gamma sequence. In some aspects, the TCR sequence comprises a CD3-epsilon sequence. In some aspects, the TCR sequence comprises a CD3-delta sequence. In some aspects, the TCR sequence comprises a CD3-zeta sequence.

### B-Cells

The disclosure provides methods of analyzing the contents and function of single B-cells including detecting proteomic and transcriptomic information. In some aspects, the disclosure provides methods and devices for identifying B-cell receptor (BCR) sequences and for identifying antibodies secreted by B-cells. In some aspects, devices of the disclosure enable detection of antibodies capable of binding specific antigens using capture beads of the disclosure configured to comprise a specific antigen of interest.

In some aspects, the antibody capture bead comprises a capture moiety comprising a polypeptide sequence encoding an antigen or epitope. In some aspects, the antibody secreted by the subject B-cell binds the antigen or epitope capture moiety. In some aspects, the antibody capture bead comprises a capture moiety comprising an antibody that binds a portion of an antibody constant region belonging to the secreted antibody. In some aspects, the antibody specific for an antibody constant region binds a portion of a heavy chain constant region or a light chain constant region.

In some aspects, the B-cell is a hybridoma, a genetically modified B-cell, a transitional B-cell, a naïve B-cell, a plasma B-cell, a B-1 cell, a B-2 cell, a regulatory B-cell, or a memory B-cell.

### B-Cell Receptors

The disclosure provides methods of identifying at least one B-cell receptor (BCR) sequence. In some aspects, the BCR sequence is encoded by an mRNA sequence.

In some aspects, the B-cell receptor sequences comprises a BCR Ig heavy chain sequence. In some aspects, the B-cell receptor sequences comprises a BCR Ig light chain sequence. In some aspects, the B-cell receptor sequences comprises a BCR Ig heavy chain variable region sequence. In some aspects, the B-cell receptor sequences comprises a BCR Ig light chain variable sequence. In some aspects, the B-cell receptor sequences comprises a BCR transmembrane domain sequence.

### T-Cell Therapeutics

The disclosure provides methods of developing therapeutics derived from TCR sequences identified in methods of the disclosure. TCR sequences derived from highly polyfunctional cells offer potential as therapeutics.

Provided are methods of creating a T-cell therapeutic comprising the at least one expressed gene or TCR sequence comprising (I) transducing at least one expressed gene or TCR sequence into a T-cell and (II) expanding the T-cell.

In some aspects, the expressed gene or TCR sequence is derived from a highly polyfunctional T-cell.

In some aspects, the polyfunctional T-cell is selected as having a PSI above a cutoff value. In some aspects, the cutoff value is a pre-determined cutoff value.

In some aspects, following transduction of the T-cell, the transduced T-cell is expanded.

In some aspects, the expanded T-cell is expanded so as to form a final cell product.

In some aspects, the final cell product is an autologous T-cell therapeutic.

### B-Cell Therapeutics

The disclosure provides methods of developing therapeutics derived from BCR sequences identified in methods of the disclosure. BCR sequences derived from highly polyfunctional cells offer several therapeutic advantages including correlating the genetic expression in the cell with the phenotype of the cell. For example, correlating the sequence of the BCR of a B-cell to the antibody secreted by the same B-cell offers valuable insight into the function of the cell.

Provided are methods of creating an B-cell therapeutic comprising the at least one expressed gene or BCR sequence comprising (I) transducing at least one expressed gene or BCR sequence into a B-cell and (II) expanding the B-cell.

In some aspects, the expressed gene or BCR sequence is derived from a highly polyfunctional B-cell.

In some aspects, the polyfunctional B-cell is selected as having a PSI above a cutoff value. In some aspects, the cutoff value is a pre-determined cutoff value.

In some aspects, following transduction of the B-cell, the transduced B-cell is expanded.

In some aspects, the expanded B-cell is expanded so as to form a final cell product.

In some aspects, the final cell product is an autologous B-cell therapeutic.

### Determining Drug Resistance Pathways in Tumor Cells

Acquired drug resistance is an unfortunately common event encountered by many patients undergoing treatment for a number of different cancers. This drug resistance, if not addressed, has significant adverse impact on the prognosis and outcome of patients. Devices and methods of the disclosure provide means to determine resistance pathways in cancer cells induced by cancer therapeutics. The determination of these pathways enables the discovery of alternate signaling pathways that can be targeted by additional cancer therapeutics, providing opportunities for improved patient outcome. Methods and devices of the disclosure provide the ability to analyze any combination of proteomic, genomic, and transcriptomic data from a single cell in a multiplexed high-throughput manner.

The disclosure provides a multiplexed method for determining a resistance pathway in a cancerous cell that confers resistance to a therapeutic agent comprising the identification of at least one nucleic acid sequence and at least one protein from a single cancer cell resistant to at least one therapeutic agent.

Resistance to a therapeutic agent can be assessed and measured using a variety of techniques. Resistance to a therapeutic agent in the cancerous cell can be measured by a reduction in efficacy of the therapeutic agent. In some aspects, resistance to a therapeutic agent in the cancerous cell is measured by a reduction in potency of the therapeutic agent. In some aspects, resistance to a therapeutic agent in the cancerous cell results in a cancerous cell that is non-responsive to treatment with the therapeutic agent. In some aspects, resistance to a therapeutic agent in the cancerous cell results in reduced cell death of a cancerous cell treated with the therapeutic agent. In some aspects, resistance to a therapeutic agent in the cancerous cell results in a patient that is non-responsive to treatment with the therapeutic agent. In some aspects, resistance to the at least one therapeutic agent results from treatment of a mammalian patient with the therapeutic agent.

Resistance to the at least one therapeutic agent can be artificially induced, induced in vitro, or induced in vivo. In some aspects, the in vitro resistance is induced by contacting the cancer cell with the at least one therapeutic agent under conditions sufficient to cause resistance.

Resistance can also be induced via gene editing techniques. Resistance can be induced by targeting a specific signaling pathway or protein known to induce resistance. In some aspects, the gene editing is a sequence-specific gene editing method. In some aspects, the gene editing technique comprises contacting the cancerous cell with a sequence-specific gene editing composition comprising a CRISPR-Cas composition, zinc-finger nuclease, TALEN, PUF, PUMBY, or meganuclease.

Resistance can be induced through gene silencing techniques. In some aspects, the gene silencing comprises contacting the cell with CRISPR-Cas composition, RNAi composition, siRNA composition, ribozyme composition, or miRNA.

In some aspects, the cancer cell is a primary tumor sample taken from a subject. In some aspects, the cancer cell is cultured cancer cell. In some aspects, the cancer cell is a circulating tumor cell. In some aspects, the cancer cell is a blood cancer cell. In some aspects, the cancer cell is a carcinoma. In some aspects, the cancer cell is a sarcoma. In some aspects, the cancer cell is a leukemia. In some aspects, the cancer cell is a lymphoma. In some aspects, the cancer cell is a myeloma. In some aspects, the cancer cell is a melanoma.

In some aspects, the method comprises providing a device of the disclosure. In some aspects, a device can comprise comprising a substrate having a plurality of chambers each comprising at least one capture bead (CB); and a surface configured to couple with a first side of the substrate to cover each chamber, wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber.

In some aspects, the capture antibody surface comprises capture antibodies specific for proteins comprising a cell signaling pathway. The cell signaling pathway can be selected from one known to be involved in conferring resistance to a specific therapeutic agent. In some aspects, the cell signaling pathway is targeted by the at least one therapeutic agent.

In some aspects, the method comprises introducing the single cancer cell to a chamber of the plurality of chambers, wherein the chamber is in fluid communication with the surface. In some aspects, adjacent chambers of the plurality of chambers each also comprise a single cancer cell. Methods of the disclosure further comprise maintaining the cancer cell in the chamber under conditions sufficient to cause cell lysis to produce a cell lysate.

Methods of the disclosure further comprise detecting the at least one protein in the cancer cell comprising the steps of: incubating the cell lysate in the chamber under conditions sufficient to allow at least one antibody and at least one protein to form an antibody:protein complex; and imaging the surface comprising the at least one antibody:protein complex, thereby identifying the one or more protein of the cancer cell. Detecting the protein of the disclosure, including visualizing the capture antibody surface can be accomplished as described in the disclosure.

Methods of the disclosure further comprise identifying the at least one nucleic acid sequence of the cancer cell comprising the steps of: incubating the cell lysate in the chamber under conditions sufficient for the at least one nucleic acid sequence to contact the CB to form an CB-nucleic acid sequence complex and determining the sequence of the complexed nucleic acid sequence. Methods and devices for capturing nucleic acid sequences and subsequently sequencing them are described throughout the disclosure.

In some aspects, identifying at least one nucleic acid sequence comprises from about one to about 1,000,000 nucleic acid sequences. In some aspects, identifying at least one nucleic acid sequence comprises from about one to about 100,000 nucleic acid sequences. In some aspects, identifying at least one nucleic acid sequence comprises from about one to about 10,000 nucleic acid sequences. In some aspects, identifying at least one nucleic acid sequence comprises from about 100 to about 10,000 nucleic acid sequences. In some aspects, identifying at least one nucleic acid sequence comprises from about 1,000 to about 100,000 nucleic acid sequences. In some aspects, identifying at least one nucleic acid sequence comprises from about 1,000 to about 1,000,000 nucleic acid sequences.

In some aspects, the at least one nucleic acid sequence is an mRNA sequence.

### Methods of Computational Correlation

Methods of the disclosure further comprise performing a computational correlation of the at least one protein and at least one nucleic acid sequence such that a pathway that confers resistance to the therapeutic agent in the cancer cell is identified.

In some aspects, the computational correlation of the at least one protein and at least one nucleic acid sequence in the subject cell is compared to a healthy cell and to a non-treated cell. In some aspects, the computational correlation of the at least one protein and at least one nucleic acid sequence in the subject cell is compared to a healthy cell. In some aspects, the computational correlation of the at least one protein and at least one nucleic acid sequence in the subject cell is compared to a non-treated cell.

Computational correlation methods of the disclosure include any suitable for analyzing large data sets of proteomic and transcriptomic information. In some aspects, the computational correlation comprises a dimensionality reduction technique. In some aspects, the dimensionality reduction technique comprises at least one of a principal component analysis (PCA), t-distributed stochastic neighbor embedding (TSME), or uniform manifold approximation and projection (UMAP).

Computational correlation methods of the disclosure are capable of identifying resistance pathways in cancerous cells and individual proteins participating in these resistance pathways. In some aspects, the computational correlation identifies an overexpression or underexpression of an individual protein or group of proteins. In some aspects, the computational correlation identifies a mutation in a gene or mRNA transcript encoding a protein that participates in the resistance pathway.

Computational correlations of the disclosure which analyze the at least one protein and at least one nucleic acid sequence can also identify compensatory pathways induced by resistance to the at least one therapeutic agent. In some aspects, the compensatory pathway is detected by the overexpression or underexpression of a single protein or group of proteins in an additional or alternate signaling pathway that compensate for the impacts to the cell induced by the original resistance pathway.

### Methods of Treating Drug Resistance

The disclosure further provides methods of treating drug resistance in a cancer cell or in a patient having cancer. Resistance pathways or compensatory pathways identified through methods of the disclosure can be targeted with one or more additional therapeutic agents designed to specifically target the identified proteins or pathways. In some aspects, the one or more additional therapeutics can be evaluated for efficacy by contacting, thereby treating, resistant cancer cells with the one or more therapeutic agents. The treated resistant cancer cells can be evaluated using devices and methods of the disclosure to further analyze changes in the proteomic, genomic, and transcriptomic profile of the treated cell. The impact of the one or more additional therapeutic agents on the identified resistance or compensatory pathways can be evaluated using devices and methods of the disclosure.

### Single-Capture Bead Multiplexed Analysis Methods

Disclosed are methods and devices for the immune profiling of a single subject cell. In some aspects, the capture bead is configured to capture mRNA for methods of monitoring transcriptomic changes or gene expression changes in a stimulated immune cell. In some aspects, the capture antibody surface comprises antibodies specific for intracellular cytokines. In some aspects, the integrated analysis of the transcriptome and cytokine secretion profile of the subject cell enables the simultaneous immunophenotyping, assessment of adaptive immune repertoire clonality and diversity, and antigen specificity. In some aspects, the subject cell is a T-cell, B-Cell, or tumor cell.

Disclosed are methods and devices for the assessment of genome-wide chromatin accessibility in a single subject cell. In some aspects, the capture bead is configured to capture accessible chromosomal DNA to identify open DNA regions in open chromatin. In some aspects, the capture antibody surface comprises antibodies specific for intracellular cytokines. In some aspects, the subject cell is a T-cell.

Disclosed are methods and devices for the immune profiling of a single subject glial cell. In some aspects, the capture bead is configured to capture mRNA for methods of central nervous system genotyping and phenotyping. In some aspects, the capture antibody surface comprises antibodies specific for intracellular cytokines.

The disclosure provides a multiplex assay chip device configured for multiplexed analysis of biological material, comprising: a plurality of capture beads (CBs), each CB having an individually unique cell barcode sequence comprising a predetermined number of base pairs and a PCR handle, a unique molecular identifier, a barcode handle sequence, and a poly T sequence/capture sequence; a substrate having a plurality of chambers, each chamber comprising an open end arranged on a first side of the substrate, a length, a width, and a depth (“dimensions”), at least one pocket arranged therein having a pocket diameter greater than the width of the chamber, wherein the at least one pocket is alternately arranged and offset from at least one pocket of an adjacent chamber and at least one of the CBs arranged within the pocket of each chamber, wherein a diameter of each CB is smaller than the pocket diameter but larger than the width of the chamber, such that, each pocket and each CB are configured such that the CB is arranged approximately in the center of each chamber; and a surface configured to couple with the first side of the substrate to cover each chamber, the surface comprising a plurality of substantially parallel lines of capture antibodies, each line of capture antibodies comprising a specific and different antibody relative to antibodies of adjacent lines of capture antibodies, wherein the antibodies are configured to bind to a different target molecule and at least one portion of each of the plurality of lines are arranged as to be exposed to each chamber.

The disclosure provides a multiplexed method for the simultaneous identification of: (a) at least one nucleic acid sequence, and (b) at least one protein from a single subject cell comprising: (I) providing a device comprising: a substrate having a plurality of chambers each comprising at least one capture bead (CB), wherein a first CB, CB1, is configured to capture a target nucleic acid; and a surface configured to couple with a first side of the substrate to cover each chamber wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber; (II) introducing the subject cell to a chamber of the plurality of chambers; (III) maintaining the subject cell in the chamber under conditions sufficient to cause cell lysis to produce a cell lysate; (IV) identifying at least one first nucleic acid sequence, comprising: (a) providing conditions sufficient for the target nucleic acid to contact the CB1 to form a CB1-target nucleic acid complex, (b) determining the sequence of the complexed target nucleic acid sequence to determine the sequence of the at least one expressed target nucleic acid sequence (V) detecting the at least one protein in the subject cell comprising the steps of: (a) incubating the cell lysate in the chamber under conditions sufficient to allow at least one antibody and at least one protein to form an antibody:protein complex; and (b) imaging the surface comprising the at least one antibody:protein complex, thereby identifying the one or more protein of the subject cell.

### Multi-Capture Bead Multiplexed Analysis Methods

Provided are methods of analyzing at least one protein and at least one nucleic acid from a single subject cell comprising a device of the disclosure configured to comprise at least two capture beads of the disclosure. In some aspects, the first and second capture bead, CB1 and CB2, are configured to both capture nucleic acids. In some aspects, CB1 and CB2 are both configured to capture proteins. In some aspects, CB1 is configured to capture nucleic acids and CB2 is configured to capture proteins. In some aspects, CB1 is configured to capture proteins and CB2 is configured to capture nucleic acids. In some aspects, CB1 and CB2 are configured to capture different cellular components.

In some aspects, CB1 is configured to capture a first target RNA sequence. In some aspects, CB2 is configured to capture a second target nucleic acid sequence that is DNA or RNA. In some aspects, the target DNA is accessible genomic DNA, autosomal DNA, chromosomal DNA, cDNA, exosome DNA, single stranded DNA, or double stranded DNA. In some aspects, the target RNA is mRNA, rRNA, tRNA, snRNA, regulatory RNA, microRNA, exosome RNA, or double stranded RNA.

In some aspects, devices of the disclosure comprise a surface coated with capture antibodies. In some aspects, the capture antibodies are specific for a class of proteins or specific signaling pathways. In some aspects, the capture antibodies are specific for phosphoproteins or cytokines.

Disclosed are methods of evaluating the results of a CRISPR-Cas screen guide RNA library in a single cell. In some aspects, CB1 is configured to capture a guide RNA from a CRISPR-Cas system. In some embodiments, CB2 is configured to capture mRNA for methods of monitoring transcriptomic changes or gene expression changes induced by the guide RNA of the CRISPR-Cas screen. In some aspects, the capture antibody surface comprises antibodies specific for phosphoproteins.

Disclosed are methods of evaluating the results of a bioproduction and synthetic biology development campaign in a single cell modified to secrete a target protein of interest. In some aspects, CB1 is configured to capture mRNA for monitoring transcriptomic changes or gene expression changes induced by the bioproduction or synthetic biology development campaign. In some embodiments, CB2 is configured to capture the target protein secreted by the modified cell. In some embodiments, the single cell is a B-cell, Chinese Hamster Ovary (CHO) cell, or bacteria cell.

### Imaging and Quantifying Proteins

Proteins captured using capture antibody surfaces of the disclosure are detected via visualization with a secondary antibody. The disclosure provides methods of detecting and quantifying proteins identified by forming complexes with capture antibodies or capture beads of the disclosure. In some aspects, following allowing the at least one capture antibody of the antibody panel or capture bead specific for the at least one protein to bind the at least one cellular protein forming at least one antibody:protein complex, the surface comprising the at least one antibody:protein complex or substrate comprising at least one capture bead:protein complex is imaged. In some aspects, imaging comprises detecting the fluorescent signal emitted by a secondary detection antibody. In some aspects, the labeled secondary antibody comprises a fluorescent, gold or silver label. In some aspects, the visualizing comprises contacting a first capture antibody:protein complex with a first labeled secondary antibody that binds the first capture antibody, contacting a second capture antibody:protein complex with a second labeled secondary antibody that binds the second capture antibody, and detecting the first labeled secondary antibody and the second labeled secondary antibody, wherein the first labeled secondary antibody and the second labeled secondary antibody each comprise a distinct label.

In some aspects, the method further comprises quantifying the at least one protein. In some aspects, the quantifying step comprises measuring an intensity and/or a density of the labeled secondary antibody. In some aspects, detecting comprises determining the signal intensity to a signal associated with each antibody:protein complex. In some aspects, the signal intensity associated with each antibody:protein complex is used to determine the level or concentration of each protein associated with each antibody:protein complex. Methods of detecting and quantifying the signal intensity of a detected antibody:protein complex, such as an antibody:protein complex is described in U.S. Pat. No. 10,584,366, the contents of which are incorporated by reference in their entirety.

### Sequencing Captured Nucleic Acid Sequences

The disclosure provides methods of sequencing target nucleic acid sequences captured by capture beads of the disclosure.

The disclosure provides methods of sequencing the individually unique cell barcode sequence of capture beads configured to capture nucleic acids. In some aspects, the individually unique cell barcode sequence is sequenced on the substrate (e.g. “on chip) after the capture bead has been placed in the chamber of the disclosure. Sequencing on chip enables sequence information to be stored and correlated with the discrete chamber of the plurality of chambers, thereby linking the chamber with the individual bead carrying the individually unique cell barcode sequence. Sequencing the individually unique cell barcode sequence comprises on chip fluorescent microscopy using fluorescently labeled nucleotide analogs.

In some aspects, sequencing the individually unique cell barcode sequence comprises synthesizing a cDNA barcode sequence. In some aspects, the cell barcode sequence can comprise any number of nucleotides. In some aspects, the cell barcode sequence comprises at least 2, at least 3, at least 4, at least 5, at least 6, at least, 7 at least, 8 at least 9, at least 10, at least 11,a t least 12, at least 13, at least 14, at least 15, at least 20, at least 25, at least 20, or at least 50 nucleotides. In some aspects, the sequence encoding the individually unique cell barcode sequence comprises 12 nucleotides. In some aspects, the sequencing is performed in the chamber. In some aspects, synthesizing the cDNA barcode sequence comprises contacting the sequence encoding the barcode handle with a primer comprising a sequence complementary to a portion of the sequence encoding the barcode handle and a polymerase, under conditions sufficient for hybridization and cDNA synthesis, wherein the contacting produces a cDNA comprising a cDNA barcode sequence. In some aspects, the conditions sufficient for hybridization and cDNA synthesis comprise a plurality of deoxynucleotides (dNTPs), wherein the least one dNTP of the plurality of deoxynucleotides (dNTPs) comprises a modification. In some aspects, each dNTP of the plurality of deoxynucleotides (dNTPs) comprises a modification. In some aspects, the modification comprises a label, wherein the label comprises a fluorophore or a chromophore. In preferred aspects, the label is a fluorescent label.

In some aspects, at least one dNTP, at least two dNTP, at least three dNTP, or at least four dNTP comprise a detectable label. In some aspects, at least one dNTP does not comprise a detectable label. In some aspects, each adenine comprises a first label, wherein each cytosine comprises a second label, each guanine comprises a third label, and each thymine comprises a fourth label. In some aspects, at least one of cytosine, guanine, adenine, and thymine does not comprises a detectable label. In some aspects, the first label, the second label, the third label, and the fourth label are distinct labels that are spectrally-distinguishable fluorescent labels.

In some aspects, sequencing the individually unique cell barcode produces a cDNA barcode sequence that is linked to the discrete chamber in which the capture bead having the individually unique cell barcode sequence originated.

In some aspects, the sequencing comprises obtaining an image following each round of dNTP addition. Each dNTP is identified by its spectrally-distinguishable fluorescent label, or in some cases, lack of a label. In some embodiments, the image produced by an unlabeled dNTP is dark (i.e. a dark state). In some aspects, successive rounds of sequencing are performed until the entire cell barcode has been sequences (i.e. each nucleotide comprising the cell barcode sequence has been sequenced via imaging).

The disclosure further provides methods of sequencing captured target nucleic acids. In some aspects, sequencing the captured target nucleic acid sequences comprises synthesizing a cDNA sequence that incorporates the individually unique cell barcode sequence and UMI into the cDNA sequence. In some aspects, sequencing the captured target nucleic acid sequences comprises synthesizing a cDNA sequence that incorporates the individually unique cell barcode sequence, or a complement sequence thereof, and UMI, or a complement sequence thereof, into the cDNA sequence. In some aspects, cDNA is synthesized from captured target nucleic acid sequences on chip and a template switch reaction is used to incorporate the individually unique cell barcode, UMI, and captured target nucleic acid sequence into one stand.

Methods for sequencing captured target nucleic acid sequences is described in U.S. patent application Ser. No. 16/349,183, the contents of which are incorporated by reference in their entirety.

CDNA sequences comprising the captured target nucleic acid sequences, or a complement sequence thereof, are amplified via PCR and used to generate libraries of nucleic acids suitable for downstream sequencing. In some aspects, the sequencing comprises next generation sequencing.

### EXAMPLES

### Example 1: T-Cell Culture Methods

T-cells of the disclosure are cultured according to the following protocol.

Jurkat cells are removed from T75 flask and displaced volume replaced with RPMI. The cells are then counted. The remaining jurkat cells are centrifuged and excess media aspirated. The cells are then resuspended in RPMI at a concentration of 1×10⁶ cells/mL. Cells are split in half for samples of stimulated and unstimulated conditions. To stimulate cells, Cell Activation Cocktail (with Brefeldin A) (500×) (Biolegend Cat #423303) is added at 1× concentration. For unstimulated cells, eBioscience™ Brefeldin A solution (1000×) (ThermoFisher Scientific Cat #00-4506-51) is added at 1× concentration. Cells are plated in a 12-well plate, 1 mL per well, and incubate at 37° C. 5% CO₂ for 5 hs. After incubation, cells are pulled from the plate and each pocket is rinsed with 1 mL of RPMI to collect remaining cells. Cells are spun down and resuspended in 1 mL of 1×PBS. Cells are spun down and resuspended in 500 μL of CellTrace™ Violet (1:500) stain and incubated for 12 min at 37° C. The stain is quenched with 1 mL of RPMI and an aliquot taken for counting. Cells are spun down and the pellet resuspended in 1×PBS at 1.5×10⁶ cells/mL. Add Protease/Phosphatase Inhibitor Cocktail (100×) (Cell Signaling Technology Cat #5872S) to each condition at 1×. Before loading stimulated sample on chip, add H₂O₂ at 0.03%. Load 35 uL into device of the disclosure that are populated with oligonucleotide beads and allow sample to wick in one third of the chip.

### Example 2: Capture Bead Substrate Preparation

The system of the disclosure (in this example, a polydimethylsiloxane (PDMS) substrate with an array of chambers containing capture beads) is prepared.

Bead loading. A 5 μL stock solution of capture beads is diluted in 35 μL of phosphate buffered saline (PBS). The bead solution is filtered through a 30 μm filter and washed with 5 mL of PBS to remove small bead fragments. The beads are then eluted from the filter and brought to a final volume of 35 μL. Prior to bead loading the substrate is plasma treated. The substrate is assembled into a flow cell with an acrylic slide and rubber gasket and secured against a bottom frame. The bead mixture is added to the flow cell and the bead suspension is mixed for an even distribution. The assembled substrate is rocked for 1.5 hrs and the flow cell is mixed every 30 mins. The mixture is then aspirated from the flow cell using an aspirator. PBS is then added to the flow cell and the assembled substrate is rocked for 30 mins. The mixture is then aspirated from the flow cell. Repeat the addition of PBS to the flow cell, rocking, and aspiration. The assembled substrate is incubated at 60° C. for 5 mins. The substrate is then disassembled from the flow cell and the substrate is kept horizontal. The substrate is incubated for 2 more mins at 60° C. Place the substrate on a spin coater and add 700 μL of agarose across the substrate ensuring that it covers the entire surface to immobilize the beads. Spin coat the substrate using the following progression: 100 rpm for 10 sec, 300 rpm for 10 sec, 500 rpm for 10 sec, 700 rpm for 10 sec, 900 rpm for 10 sec, 1500 rpm for 10 sec, 2000 rpm for 10 sec, and 2500 rpm for 20 sec. The substrate is then dried for 1 hr. The chambers are then rehydrated with 5 mL of PBS. Excess PBS is removed by tilting the substrate. The substrate is then incubated for 5 mins at 60° C. followed by a 30 min rest at room temperature.

### Example 3: On-Device Barcode Reading of an Individually Unique Barcode Sequence of a Capture Bead

The unique barcode sequence of each capture bead of the array of chambers containing capture beads is read on device. See FIGS. 2B and 2C.

Pre-hybridize the beads with sequencing primer at 20 uM final concentration in PBS with 0.1% triton at a 40 μL volume and incubate for 5 min at room temperature, mixing once during incubation. Remove primer solution and flush twice with 100 μL Post Extension Buffer (PEB) (Table 3).

Prepare 50 uL of fluorescent sequencing master mix according to Table 1. Slowly dispense 45 μL of fluorescent reaction mix into the flow cell and incubate 5 minutes at room temperature. Transfer to an incubator for 20 min at 60° and mix gently every 5 mins. Wash once slowly with 90 uL PEB and remove buffer.

TABLE 1
Fluorescent sequencing master mix

| Reagent | 1 chip | 2 chips | 3 chips | 4 chips |
|---|---|---|---|---|
| Thermopol buffer (10X) | 5.0 | 10.0 | 15.0 | 20.0 |
| 200 mM KCl | 2.5 | 5.0 | 7.5 | 10.0 |
| Post Extension Buffer | 10.0 | 20.0 | 30.0 | 40.0 |
| 0.1M NaOH | 6.0 | 12.0 | 18.0 | 24.0 |
| 488A | 4 | 8 | 12 | 16 |
| 647T (1:10 dilute)* | 0.5 | 1.0 | 1.5 | 2.0 |
| 488C | 0.3 | 0.6 | 0.9 | 1.2 |
| 647C | 0.25 | 0.50 | 0.75 | 1.00 |
| fill G | 0.5 | 1.0 | 1.5 | 2.0 |
| 25 mM MgCl2 | 14.0 | 28.0 | 42.0 | 56.0 |
| Therminator X polymerase | 0.5 | 1.0 | 1.5 | 2.0 |
| water | 4.5 | 8.9 | 13.4 | 17.8 |

*Use 1:15 dilution of 647T for 1^{st} cycle

Prepare 200 μL “Fill-in” reversible terminator master mix according to Table 2. Slowly dispense 45 μL of “fill in” reaction mix into the flow cell of the device containing the substrate with chambers containing capture beads and incubate for 5 min at room temperature. Transfer to an incubator for 20 min at 60° C. and mix gently every 5 min. Remove remaining reaction mixture and flush 2× with 90 μL PWB (Table 4), flush 1× with 90 μL PEB, and flush 1× with 90 μL PEB with BSA.

TABLE 2
Fill-in″ reversible terminator master mix

| Reagent | 1 chip | 2 chips | 3 chips | 4 chips |
|---|---|---|---|---|
| Thermopol buffer (10X) | 5.0 | 10 | 15 | 20 |
| 200 mM KCl | 2.5 | 5 | 7.5 | 10 |
| Post Extension Buffer | 10.0 | 20 | 30 | 40 |
| 25 mM MgCl2 | 10.5 | 21 | 31.5 | 42 |
| 0.1M NaOH | 3.5 | 7 | 10.5 | 14 |
| fill ins* | 16.0 | 32 | 48 | 64 |
| Standard Therminator polymerase | 1.0 | 2 | 3 | 4 |
| Water | 1.5 | 3 | 4.5 | 6 |

*Volume is for total fill ins, divide the volume by 4 to get the volume per nucleotide

Image the device containing the substrate with chambers containing capture beads using bright field, 405 nm, 488 nm, 555, nm and 647 nm filters. Exposure time for 405 nm, 488 nm, 555 nm, and 647 nm may need to be lowered depending on bead signal. In some aspects, the wavelength of each filter is ±3 nm.

Save the image for analysis. Each bead is given a location (chamber 1, chamber 2, chamber 3, etc.) Software determines nucleotide call based on fluorescent signal intensity of bead signal and records nucleotide call and bead location. Over the course of several cycles, the barcode sequence of each bead is determined and linked to their chamber location. Cleave 3′ reversible terminator with Buffered Sodium Nitrite. Buffered Nitrite solution formula: 700 mM NaNO2 from powder using 1M NaOAc ph5.5 and 0.1% Triton X. Check pH and adjust as necessary. 1 mL NaOAc, 1 uL Triton X, 50 mg Sodium nitrite. Gently dispense 90 μL buffered Nitrite solution into the flow cell and incubate at room temp for 5 min, mixing halfway through incubation, then remove solution. Repeat the addition of 90 μL buffered nitrite solution and incubation two additional times. Wash once with 90 μL PEB and remove buffer.

Cleave fluorescent molecules with 1% sodium periodate. Gently dispense 90 μL sodium periodate solution into the flow cell and incubate at room temperature for 3 min, mixing halfway through incubation, followed by removal of the solution. Wash twice with 90 μL PWB, and twice with PEB, removing solution after each wash.

Repeat the protocol for each nucleotide of the barcode sequence, beginning at the addition of the fluorescent sequencing master mix to the device and ending with cleaving the fluorescent molecule with sodium periodate. Thus, for a 10 nucleotide barcode sequence, 10 cycles will be performed.

After barcode reading is complete and sequencing data is obtained, software must link the gene expression data for each barcode so that the mRNA expressed in an individual cell is connected to the physical location on the chip.

TABLE 3
PEB components

| Reagent | Volume for 25 μL | |
|---|---|---|
| 1M tris pH 8 | 1250 | μL |
| 30% MeONH2 | 83.3 | μL |
| 1M NaOH | 25 | μL |
| 100% Triton X | 25 | μL |
| water | 23.6 | mL |

TABLE 4
PWB components

| Reagent | Volume for 25 mL | |
|---|---|---|
| 1M tris pH 7.5 | 1250 | μL |
| 0.5M EDTA | 25 | μL |
| 30% MeONH2 | 83.3 | μL |
| 5M NaCl | 5 | mL |
| 100% Triton X | 125 | μL |
| water | 18.6 | mL |

### Example 4: Protein Detection

Cellular proteins can be detected according utilizing capture antibody surfaces as described throughout the disclosure.

Cultured cells treated with protease/phosphatase inhibitor cocktail (Cell Signaling Technology Cat #5872S (100×)) at 1× concentration and 0.03% hydrogen peroxide are loaded onto the substrate in a 35 μL addition and allowed to wick into a third of the chambers.

The Assembled device is loaded into an imaging system (such as a fluorescence microscope or IsoLight) and the device is clamped using a cell clamping script. Lyse cells by flowing lysis buffer through device at 0.1× concentration (Cell Signaling Technology Cat #9803) with NxGen™ RNase Inhibitor (VWR Cat #97065-224) (1:1000). Lift cell clamps at reduced speed and clamp at regular clamping steps. In Process Runner manually focus chips for cells/wells imaging. After cells/well imaging, incubate chips at RT overnight. Following incubation, run fluidics backend with extra PBS rinse. Manually focus chips and perform signal imaging. After signal imaging, home all motors and remove chips for reverse transcription. Replace with cleaning chips and clean IsoLight. Generate proteomic data using proteomic analysis software (IsoSpeak).

### Example 5: Generating cDNA Library from Captured mRNA

Captured mRNA sequences are synthesized into cDNA sequences containing the captured mRNA, cell barcode sequence, and unique molecular identifier.

The assembled device containing the substrate with the array of chambers and capture beads is removed from the IsoLight device. Any remaining liquid is removed from the device via aspiration. The flow cell of the device is washed twice with 500 μL of PBS and removed followed by a single wash with 200 μL of RT buffer, which is removed following washing. Reverse transcription mixture according to Table 5 is applied to the flow cell in a 40 μL or 200 μL addition and mixed via pipette. The device is placed on to a thermal cycler and incubated for 10 min at 25° C. followed by incubation for 90 min at 45° C. The device is washed three times with 500 μL of PBS.

TABLE 5
Reverse Transcription Mixture

| Reagent | Volume (μL) |
|---|---|
| 5x Maxima RT buffer | 7.7 |
| 20% Ficol | 7.7 |
| 10 mM dNTPs | 3.8 |
| Rnase inhibitor | 1.0 |
| 100 uM Template switch oligo | 1.9 |
| Reverse Transcriptase | 1.9 |
| Water | 4.8 |
| extra 5x RT buffer | 9.6 |

### Example 6: Removing cDNA from Immobilized Capture Beads

Synthesized cDNA is removed from the chambers containing capture beads without disturbing or removing the capture beads.

Flush the device using two 300 μL additions of PBS aspirating PBS from the device after each addition. Dispense 40 μL of 60% DMSO in water into the flow cell and mix via pipetting. Incubate for 2 mins. Pipette to mix and collect sample into 1.5 mL tube. Add 20 μL of PBS to each tube. The DNA is cleaned and purified utilizing Solid Phase Reversible Immobilization (SPRI) beads from Beckman Coulter. Clean up is done according to Beckman Coulter Ampure XP manufacturer's protocol. Following clean up the resultant cDNA mixture comprises the cDNA generated in each chamber of the plurality of chambers.

### Example 7: Amplification of cDNA Library

The cDNA mixture is amplified utilizing two PCR steps to form a cDNA library.

The eluted cDNA (10 μL volume) following SPRI clean up is amplified utilizing a PCR mixture according to Table 6. A 40 μL volume of PCR mixture is added to the cDNA sample and mixed. The thermal cycler is run according to the protocol in Table 7. Perform a 0.6×SPRI clean up to capture cDNA and concentrate into a smaller volume per manufacture protocol.

TABLE 6
PCR mix

| Reagent | μL |
|---|---|
| Jump Start mix (2x) | 25 |
| 100 uM SMART PCR primer | 0.8 |
| 100 uM primer 23′ | 0.8 |
| SPRI elution (cDNA) | 10 |
| water | 13.4 |

TABLE 7
Step 1 PCR Cycle

| Step | Temperature | Time | |
|---|---|---|---|
| 1 | 95° C. | 3 | min |
| 2 | 98° C. | 20 | sec |
| 3 | 65° C. | 45 | sec |
| 4 | 72° C. | 3 | min |

| | |
|---|---|
| 5 | go to step 2 for 4 cycles |

| | | | |
|---|---|---|---|
| 6 | 98° C. | 20 | sec |
| 7 | 67° C. | 20 | sec |
| 8 | 72° C. | 3 | min |

| | |
|---|---|
| 9 | go to step 6 for 8 cycles |

| | | | |
|---|---|---|---|
| 10 | 72° C. | 5 | min |

Hold at 10° C.

A second round of PCR is conducted to generate the cDNA library. The eluted cDNA (10 μL volume) following SPRI clean up is amplified utilizing a PCR mixture according to Table 6. A 40 μL volume of PCR mixture is added to the cDNA sample and mixed. The thermal cycler is run according to the protocol in Table 8. Perform a 1×SPRI clean up to capture cDNA and concentrate into a smaller volume per manufacture protocol.

TABLE 8
Step 2 PCR Cycle

| Step | Temperature | Time | |
|---|---|---|---|
| 1 | 94° C. | 2 | min |
| 2 | 94° C. | 30 | sec |
| 3 | 55° C. | 30 | sec |
| 4 | 72° C. | 2 | min |
| 5 | go to step 2 repeat 21X | | |
| 6 | 72° C. | 5 | min |

hold at 10° C.

The amplified cDNA is run on an Agilent High Sensitivity DNA BioAnalyzer chip (in this example) using a High Sensitivity DNA kit, per manufacturer protocol. The library is now ready for sequencing.

### Example 8: Multiplexed and Simultaneous Single Cell Transcriptomic and Proteomic Detection from Single Hela and Monocyte Cells

Using devices and methods disclosed herein, single Hela and monocyte cell were analyzed to simultaneously measure protein and gene expression levels from a same single cell.

Monocytes and Hela cells were combined onto a single device of the disclosure and were processed according to methods of the disclosure. After cells were loaded onto the device, protein data was used to initially cluster the data into distinct subpopulations of cells with significant differences in heterogeneous protein expression. Differentially expressed genes and gene pathways were then identified across these subpopulations. This mitigated concerns regarding visual confirmation of determining which chamber contained which cell type.

Protein expression for individual single cells was measured using barcode-based proteins detection methods described herein. A heat map and hierarchical cluster of the proteomic data set was generated. FIG. 24 is a heat map of protein expression and hierarchical cluster of 73 single Hela and monocyte cells from the cell sample. Each column of the map represents a single cell. Cells were matched to their location based on the bead cell barcode sequence. The cell barcode sequence was sequenced according to methods of the disclosure, wherein the barcode is sequenced on chip as described in Example 3. The sequence was verified via downstream sequencing methods such as Illumina-based sequencing. High protein expression is indicated in red while low protein expression is indicated in blue. Each protein is indicated in a separate row. FIG. 25 is a heat map of protein expression and hierarchical cluster of 45 single Hela and monocyte cells from the cell sample. Each column of the map represents a single cell. Cells were matched to their location based on the cell barcode sequence. High protein is indicated in red while low protein expression is indicated in blue. Each protein is indicated in a separate row.

Gene expression analysis was performed according to methods described herein. Bead-based mRNA capture was performed according to methods described herein. A heat map and hierarchical cluster of the gene expression data set was generated. FIG. 26 is a heat map of raw gene expression and hierarchical cluster of 45 single Hela and monocyte cells from the cell sample. Each column of the map represents a single cell. Cells were matched to their location based on the cell barcode sequence. High gene expression is indicated in red while low gene expression is indicated in blue. Each gene is indicated in a separate row. FIG. 27 is a heat map of normalized and logged gene expression and hierarchical cluster of 45 single Hela and monocyte cells from the cell sample. Each column of the map represents a single cell. Cells were matched to their location based on the cell barcode sequence. High gene expression is indicated in red while low gene expression is indicated in blue. Each gene is indicated in a separate row.

A Uniform Manifold Approximation and Projection (UMAP) was applied to the gene expression data for the 45 matched single cells. UMAP is a dimension reduction technique that allows for visualization of variance between data points. The Leiden algorithm was applied to cluster and color the data. The Leiden algorithm was applied to uncover community structure in large complex datasets. FIGS. 28A and 28B revealed two subgroups of cells, subgroup 0 and subgroup 1. FIG. 29 displays the expression of top 50 marker genes for each of the two clusters calculated by the Leiden algorithm, subgroup 0 and subgroup 1. The 100 columns represent the respective top 50 marker genes for each of the two clusters. The rows represent the cells and are coded by which cluster they were calculated to be from, subgroup 0 or subgroup 1.

FIG. 30 is a heat map and hierarchical cluster of the top 1000 most variable genes. Subgroup 0 is shaded with diagonal lines and subgroup 1 is shaded. High gene expression is indicated in red while low gene expression is indicated in blue.

FIG. 31A is a violin plot of RPS6 gene expression for subgroup 0 and subgroup 1. Each dot represents a unique cell from our matched 45 cells.

FIG. 31B is a violin plot of MIP-1-Beta gene expression for subgroup 0 and subgroup 1. Each dot represents a unique cell from our matched 45 cells.

FIG. 31C is a violin plot of beta actin gene expression for subgroup 0 and subgroup 1. Each dot represents a unique cell from our matched 45 cells.

Protein expression and gene expression data was obtained from the same single Hela or monocyte cells. Further, two separate cell populations/clusters with protein and sequencing data was obtained (FIGS. 28-31). The analysis of individual genes from the gene expression data show different levels of coverage.

### Example 9: Multiplexed and Simultaneous Single Cell Transcriptomic and Proteomic Detection

Transcriptome data alone does not fully characterize cellular function. Linking single cell transcriptomics data with its corresponding single-cell functional proteomic information from the same single cell enables a nuanced understanding of cell biology including immune biology and tumor biology by revealing connections between gene expression and subsequent protein expression. Cells are heterogenous and subsets of cells from a bulk sample may orchestrate activity missed in bulk cell analysis.

Using devices and methods disclosed herein, 55 single melanoma tumor cells were analyzed to measure protein and gene expression levels. Protein data was used to initially cluster the data into distinct subpopulations of cells with significant differences in heterogeneous protein expression. Differentially expressed genes and gene pathways were then identified across these subpopulations. This analysis enables one to identify which specific genes and gene pathways can have a downstream impact on the protein heterogeneity of an analyzed sample.

The melanoma cells were loaded onto the device of the disclosure comprising a substrate comprising an array of chambers each comprising an mRNA capture bead. The substrate was coupled to a surface comprising an array capture antibodies arranges in substantially parallel lines. The antibody array comprised capture antibodies specific for phosphoproteins including alpha tubulin, cleaved PARP, P-elF4E, P-IkBA, P-MEK1-2, P-Met, P-NF-kB p65, P-p44-42 MAPK, P-p90RSK, P-PRAS40, p-Rb, P-s6 Ribosomal, p-Stat 1, p-Stat 3, and p-Stat 5. Protein detection was performed according to methods disclosed herein. Transcriptomic analysis was performed according to methods disclosed herein including mRNA capture, cDNA synthesis, and downstream sequencing.

A two-dimensional (2D) t-Distributed Stochastic Neighbor Embedding (t-SNE) analysis of protein intensity was performed using the proteomic data collected for the melanoma cells (FIG. 32A). Two subsets of cells were identified, subset 1 having low functional heterogeneity and subset 2, having high functional protein heterogeneity. The 2D t-SNE analysis of each individual phosphoprotein is displayed in FIG. 32B with low protein intensity indicated in blue and high protein intensity indicated in red. The individual protein analysis of FIG. 32B reveals the same subsets of single cells as in FIG. 32A.

The two cell subsets, subset 1 and subset 2, have significant differences in protein heterogeneity (FIG. 33A). Heterogeneity is defined as the percentage of single cells in the sample expressing two or more proteins simultaneously. In this case, it is the number of co-expressed phosphoproteins. For cells to have high protein heterogeneity means that multiple proteins were expressed simultaneously by those cells. Subset 1 has low functional heterogeneity and subset 2 has high functional heterogeneity. In subset 2, cells expressed 2, 3, 4, 5, or more than 5 proteins. Further, there are differences in the Functional Heterogenity index (FHI) of the two subsets. FIG. 33B details the differences in FHI. The Functional Heterogeneity Index is an index factoring both heterogeneity and signal intensity of the proteins expressed in each single cell. Subsets of cells that have higher degrees of heterogeneity may be linked to drug-resistance mechanisms.

A heat map and hierarchical cluster of the proteomic data set was generated (FIG. 34). The heat map and hierarchical clustering was generated utilizing pheatmap, an R package. Hierarchical Clustering is an algorithm that groups similar objects into groups called clusters. The endpoint is a set of clusters, where each cluster is distinct from each other cluster, and the objects within each cluster are broadly similar to each other. Clustering was performed on the data matrix based on the Euclidean distances of the single cell profiles. See https://cran.r-project.org/web/packages/pheatmap/index.html. Subset 1, the low functional heterogeneity set, is indicated in shading with diagonal lines. Subset 2, the high functional heterogeneity set, is indicated in plain shading. Each row of the map depicts a distinct protein. High protein expression is indicated by red shading while low protein expression is indicated by blue shading. Subset 2 is characterized by higher average protein expression intensity of the 15 protein panel while subset 1 is characterized by low average protein intensity for the same 15-proteins. FIG. 39 is a table detailing proteins with statistically significant differences between the two cell populations, subset 1 and subset 2.

A heat map and hierarchical cluster was also generated for the single cell transcriptomic sequencing data (FIG. 35). Subset 1, the low functional heterogeneity set, is indicated in shading with diagonal lines. Subset 2, the high functional heterogeneity set, is indicated in plain shading. Each row of the map depicts a distinct gene. The map depicts the top 100 statistically significant gene expression differences between the two cell subsets. High gene expression is indicated by red shading while low gene expression is indicated by blue shading. Hierarchical clustering based on top 100 statistically significant gene expression differences between two cell subsets was used to generate the heat map. Genes most strongly associated (p<0.01) with the high protein heterogeneity subset 2 included MGAT4B, MRPL23, ACAA2, RAD21, VPS28, JTB, SYNGR2, KAT6A, IRGQ, and MAPKAPK3 (FIG. 36). Therefore, a subset of genes seem to be directly regulating the phosphoproteins upregulated in subset 2 and there may be key gene networks driving these malignant melanoma cells. Genes most strongly associated (p<0.01) with the low protein heterogeneity subset 1 included CCNI, ATP5MC3, ANKRD12, EZR, PRRC2C, GBA, DNAJC25-GNG10, GNG10, NUDTS, and AKAP11 (FIG. 36). Table 9 is a table detailing genes with statistically significant differences in expression between the two cell populations, subset 1 and subset 2.

TABLE 9
genes with statistically significant differences in expression between
the two cell populations, subset 1 and subset 2.

| Rank | P Value | Gene |
|---|---|---|
| 1 | 0.000241 | CCNI |
| 2 | 0.000735 | ATP5MC3 |
| 3 | 0.00331 | ANKRD12 |
| 4 | 0.003401 | MGAT4B |
| 5 | 0.003501 | EZR |
| 6 | 0.003714 | PRRC2C |
| 7 | 0.003988 | GBA |
| 8 | 0.004257 | DNAJC25- |
| | | GNG10 |
| 9 | 0.004257 | GNG10 |
| 10 | 0.004623 | MRPL23 |
| 11 | 0.004906 | NUDT5 |
| 12 | 0.005109 | AKAP11 |
| 13 | 0.005666 | ITGA6 |
| 14 | 0.005806 | ACAA2 |
| 15 | 0.005821 | GPD2 |
| 16 | 0.006014 | RAD21 |
| 17 | 0.006021 | CMC2 |
| 18 | 0.007021 | SGPL1 |
| 19 | 0.007096 | CAPZA2 |
| 20 | 0.007324 | VPS28 |
| 21 | 0.007532 | ENSG- |
| | | 00000261236.8 |
| 22 | 0.007614 | JTB |
| 23 | 0.00765 | PTPN9 |
| 24 | 0.008033 | SYNGR2 |
| 25 | 0.008108 | KAT6A |
| 26 | 0.008303 | IRGQ |
| 27 | 0.008324 | MAPKAPK3 |
| 28 | 0.008443 | EPB41L4A- |
| | | AS1 |
| 29 | 0.008475 | DHX29 |
| 30 | 0.008812 | SERPINE2 |
| 31 | 0.008859 | GAPDH |
| 32 | 0.009147 | PRPF31 |
| 33 | 0.009609 | ENSG- |
| | | 00000284526.1 |
| 34 | 0.009637 | PCYTIA |
| 35 | 0.010294 | TK1 |
| 36 | 0.010782 | GSTM3 |
| 37 | 0.011012 | CENPN |
| 38 | 0.011216 | PIGS |
| 39 | 0.011326 | RPL24 |
| 40 | 0.011924 | RPL4 |
| 41 | 0.01269 | PIP4K2A |
| 42 | 0.01303 | GDF11 |
| 43 | 0.013152 | MRPL11 |
| 44 | 0.013311 | RAB10 |
| 45 | 0.013464 | HLA-A |
| 46 | 0.013496 | BRI3 |
| 47 | 0.013593 | CD81 |
| 48 | 0.013795 | SECISBP2 |
| 49 | 0.013868 | ALDOA_ENSG- |
| | | 00000149925.22 |
| 50 | 0.014003 | CCND1 |
| 51 | 0.014144 | HM13 |
| 52 | 0.014821 | PUF60 |
| 53 | 0.015396 | PYCR3 |
| 54 | 0.015549 | TRPT1 |
| 55 | 0.015562 | CCDC6 |
| 56 | 0.015697 | TAOK3 |
| 57 | 0.016191 | PSAP |
| 58 | 0.016228 | UBA3 |
| 59 | 0.016289 | ELAVL1 |
| 60 | 0.016526 | MPRIP |
| 61 | 0.016648 | DTL |
| 62 | 0.016888 | KDELR1 |
| 63 | 0.016913 | PCOLCE |
| 64 | 0.017334 | SLC35E2B |
| 65 | 0.017404 | RPS12 |
| 66 | 0.017591 | NDUFS6 |
| 67 | 0.018753 | POLR3G |
| 68 | 0.019481 | PRPF6 |
| 69 | 0.019587 | IFT22 |
| 70 | 0.019932 | SPATS1 |
| 71 | 0.020004 | ZNF664 |
| 72 | 0.020588 | NUBP2 |
| 73 | 0.020888 | LPP |
| 74 | 0.020948 | NDUFA7 |
| 75 | 0.021175 | RTTN |
| 76 | 0.021632 | DYNC112 |
| 77 | 0.021956 | nan-56 |
| 78 | 0.022304 | SNTA1 |
| 79 | 0.02234 | RRAGB |
| 80 | 0.022502 | PUM3 |
| 81 | 0.022575 | MRPL47 |
| 82 | 0.023056 | GOLGA4 |
| 83 | 0.023166 | FADS1 |
| 84 | 0.023427 | ENSG- |
| | | 00000269482.1 |
| 85 | 0.023538 | GLMP |
| 86 | 0.023578 | PPPIR11 |
| 87 | 0.023668 | TCF19 |
| 88 | 0.023761 | XRCC2 |
| 89 | 0.023907 | ANAPC10 |
| 90 | 0.024085 | RRM1 |
| 91 | 0.024484 | ENSG- |
| | | 00000279800.2 |
| 92 | 0.024814 | CC2D1B |
| 93 | 0.024855 | FEZ2 |
| 94 | 0.025082 | CLMN |
| 95 | 0.025104 | NUP58 |
| 96 | 0.02511 | MYBBPIA |
| 97 | 0.025113 | WDR74 |
| 98 | 0.025162 | KIAA1522 |
| 99 | 0.025194 | SDE2 |
| 100 | 0.025577 | KCNN2 |
| 101 | 0.025708 | ADRA2C |
| 102 | 0.025721 | TBC1D23 |
| 103 | 0.025727 | RIC1 |
| 104 | 0.025873 | nan |
| 105 | 0.025873 | ENSG- |
| | | 00000283580.3 |
| 106 | 0.025972 | ELOF1 |
| 107 | 0.025977 | TOX4 |
| 108 | 0.026067 | CTR9 |
| 109 | 0.026264 | CCSAP |
| 110 | 0.02639 | BLVRB |
| 111 | 0.026496 | TOMM70 |
| 112 | 0.026523 | ZNF614 |
| 113 | 0.026563 | ENSG- |
| | | 00000173867.10 |
| 114 | 0.02677 | EDF1 |
| 115 | 0.027166 | PPP6R2 |
| 116 | 0.027167 | NABP2 |
| 117 | 0.02719 | RPS11 |
| 118 | 0.027303 | TMEM39A |
| 119 | 0.027541 | CABLES1 |
| 120 | 0.027743 | RAB13 |
| 121 | 0.028109 | MIDN |
| 122 | 0.028453 | KDM4A-AS1 |
| 123 | 0.028484 | SH3BP4 |
| 124 | 0.028598 | LASIL |
| 125 | 0.028663 | KIAA0319L |
| 126 | 0.028686 | TUG1 |
| 127 | 0.028988 | KREMEN1 |
| 128 | 0.029009 | MTX2 |
| 129 | 0.029364 | HMG20B |
| 130 | 0.029557 | TRIM33 |
| 131 | 0.029613 | ENSG- |
| | | 00000219200.12 |
| 132 | 0.029689 | HADH |
| 133 | 0.030277 | MTFR1L |
| 134 | 0.030524 | NRAS |
| 135 | 0.03057 | RPF1 |
| 136 | 0.031028 | NMB |
| 137 | 0.031139 | RPL23AP42 |
| 138 | 0.031616 | CDK5RAP3 |
| 139 | 0.031721 | BUB3 |
| 140 | 0.031909 | AGO1 |
| 141 | 0.031957 | BCAS2 |
| 142 | 0.032065 | ZNF704 |
| 143 | 0.032079 | ZMAT5 |
| 144 | 0.03215 | GLRX3 |
| 145 | 0.032323 | IPO4 |
| 146 | 0.033021 | SRSF7 |
| 147 | 0.033378 | SSR2 |
| 148 | 0.033831 | PTBP3 |
| 149 | 0.034002 | CMIP |
| 150 | 0.034246 | CPNE1 |
| 151 | 0.034268 | DHX40 |
| 152 | 0.034358 | EIF2S2 |
| 153 | 0.034442 | TSPAN17 |
| 154 | 0.035023 | TAOK1 |
| 155 | 0.035124 | PLIN2 |
| 156 | 0.035302 | KLHL24 |
| 157 | 0.035405 | ENSG- |
| | | 00000279091.1 |
| 158 | 0.035544 | NASP |
| 159 | 0.035548 | CPSF4 |
| 160 | 0.035754 | MRPL32 |
| 161 | 0.035827 | ENSG- |
| | | 00000285043.2 |
| 162 | 0.035874 | ENSG- |
| | | 00000286905.1 |
| 163 | 0.03588 | TCP1 |
| 164 | 0.036076 | SNORA67 |
| 165 | 0.0366 | TMEM141 |
| 166 | 0.037119 | ST13P19 |
| 167 | 0.03713 | GBAP1 |
| 168 | 0.037392 | DESI1 |
| 169 | 0.037428 | DBNL |
| 170 | 0.037494 | PGAM4 |
| 171 | 0.037759 | GOSR1 |
| 172 | 0.037818 | FAM86DP |
| 173 | 0.03824 | HMG20A |
| 174 | 0.038481 | DHFRP1 |
| 175 | 0.039181 | KRR1 |
| 176 | 0.039214 | NLRC5 |
| 177 | 0.03946 | TUBB |
| 178 | 0.039555 | SLC35B2 |
| 179 | 0.039637 | PGRMC1 |
| 180 | 0.04083 | PPP1R3B |
| 181 | 0.040961 | DAXX |
| 182 | 0.041983 | HNRNPK |
| 183 | 0.042145 | TMEM219 |
| 184 | 0.042638 | GDI1 |
| 185 | 0.04273 | YY1 |
| 186 | 0.042851 | HACD3 |
| 187 | 0.043445 | ENSG- |
| | | 00000285723.1 |
| 188 | 0.043831 | CDK11B |
| 189 | 0.044168 | ZFAND5 |
| 190 | 0.044224 | INTS8 |
| 191 | 0.044228 | TSPAN14 |
| 192 | 0.044706 | RRP1B |
| 193 | 0.044728 | RAD51 |
| 194 | 0.04476 | WAC |
| 195 | 0.044895 | DENND5A |
| 196 | 0.045012 | ZNF827 |
| 197 | 0.045054 | TMTC1 |
| 198 | 0.045167 | FUS |
| 199 | 0.04537 | Clorf174 |
| 200 | 0.045469 | H2AX |
| 201 | 0.045558 | ENSG- |
| | | 00000287833.1 |
| 202 | 0.04563 | DCPS |
| 203 | 0.045716 | CLEC16A |
| 204 | 0.045726 | BLOC1S6P1 |
| 205 | 0.046019 | RPF2 |
| 206 | 0.046091 | PAIP1 |
| 207 | 0.046236 | PNPLA6 |
| 208 | 0.046399 | DZIP3 |
| 209 | 0.046403 | SPEF2 |
| 210 | 0.046566 | PFKP |
| 211 | 0.046664 | COX8A |
| 212 | 0.046687 | UAP1L1 |
| 213 | 0.04683 | MED16 |
| 214 | 0.046843 | SEC22A |
| 215 | 0.046874 | CEP170B |
| 216 | 0.04693 | NMT1 |
| 217 | 0.047166 | NOB1 |
| 218 | 0.047264 | TRAM2-AS1 |
| 219 | 0.047688 | PDCD6IPP2 |
| 220 | 0.047692 | C18orf21 |
| 221 | 0.047713 | SEMA3E |
| 222 | 0.047765 | ERLEC1 |
| 223 | 0.047812 | HSF1 |
| 224 | 0.047815 | ENSG- |
| | | 00000270112.4 |
| 225 | 0.04784 | GORASP1 |
| 226 | 0.047869 | ESCO1 |
| 227 | 0.047988 | PITPNA-AS1 |
| 228 | 0.048129 | ST6GALNAC2 |
| 229 | 0.048139 | EML4 |
| 230 | 0.048156 | SRP72 |
| 231 | 0.048234 | ENSG- |
| | | 00000271853.5 |
| 232 | 0.048348 | UNC5B |
| 233 | 0.048392 | ENSG- |
| | | 00000272442.2 |
| 234 | 0.048559 | WSB1 |
| 235 | 0.048611 | NCAPG |
| 236 | 0.048707 | DHX32 |
| 237 | 0.048969 | UBL5 |
| 238 | 0.049001 | ENSG- |
| | | 00000265566.2 |
| 239 | 0.049009 | POLR2I |
| 240 | 0.049047 | RALGPS2 |
| 241 | 0.049357 | MEIS2 |
| 242 | 0.049462 | MRPL55 |
| 243 | 0.049621 | C3orf14 |
| 244 | 0.049654 | PKN3 |
| 245 | 0.049922 | RCL1 |
| 246 | 0.049924 | MAGEA1 |
| 247 | 0.049934 | MACROH2A2 |
| 248 | 0.049958 | BAGE2 |
| 249 | 0.049973 | PDZD11 |

A 2D t-SNE plot was generated for the key identified genes (FIG. 38). Statistically upregulated genes in subset 1 include CCNI, ATP5MC3, ANKRD12, EZR, and PRRC2C. Statistically upregulated genes in subset 2 include MGAT4B, MRPL23, ACAA2, RAD21, and VPS28. The first five plots (first row) visually indicate the increased expression levels of the corresponding genes in cell subset 1, whereas the next five plots (second row) indicate increased expression levels in cell subset 2.

Next, a gene pathway analysis was performed to determine the connections between variation in gene expression in the two subsets and the downstream effect on protein expression. Genes were assigned to predefined bins based on their functional characteristics. Pathway Analysis was performed on both the significantly Up Regulated and Down Regulated genes utilizing EnrichR, a published tool for doing enrichment analysis for a list of input genes against a database of 102 curated gene set libraries. Pathway Analysis is utilized to see over representation of specific pathways in our list of significantly differentiated genes. See Kuleshov et al. Nucleic Acids Res. 2016 Jul. 8; 44: W90-W97. Gene ontology (GO) terms with differential expression in the two cell subpopulations are shown in Table 10, along with the specific genes that were differentially expressed within that GO term. Statistical test included Bonferroni correction (adjusted p values). With adjusted p values, all statistically significant pathways increased in expression in the high heterogeneity cell subset, subset 2.

TABLE 10
Gene pathway analysis of 55 melanoma single cell sample

| | | Adjusted | | Log2Fold |
|---|---|---|---|---|
| Term | P-value | P-value | Genes | Change |
| Mitochondrial | 0.00009 | 0.01117 | MRPL47; MRPL23; | Positive |
| translation | | | MRPL32; MRPL55; | |
| initiation Homo | | | MRPL11 | |
| sapiens R-HSA- | | | | |
| 5368286 | | | | |
| Mitochondrial | 0.00009 | 0.01117 | MRPL47; MRPL23; | Positive |
| translation | | | MRPL32; MRPL55; | |
| termination Homo | | | MRPL11 | |
| sapiens R-HSA- | | | | |
| 5419276 | | | | |
| Mitochondrial | 0.00009 | 0.01117 | MRPL47; MRPL23; | Positive |
| translation | | | MRPL32; MRPL55; | |
| elongation | | | MRPL11 | |
| Homo sapiens R- | | | | |
| HSA-5389840 | | | | |
| Mitochondrial | 0.00013 | 0.01117 | MRPL47; MRPL23; | Positive |
| translation Homo | | | MRPL32; MRPL55; | |
| sapiens R-HSA- | | | MRPL11 | |
| 5368287 | | | | |
| mRNA Splicing | 0.00013 | 0.01117 | FUS; ZMAT5; | Positive |
| Homo sapiens | | | PRPF31; POLR2I; | |
| R-HSA-72172 | | | SRSF7; ELAVL1 | |
| MASTL human | 0.00022 | 0.01310 | DHFRP1; RRM1; | Positive |
| kinase ARCHS4 | | | NASP; TCP1; | |
| coexpression | | | NCAPG; CENPN; | |
| | | | RRP1B; SRSF7 | |
| VRK1 human | 0.00022 | 0.01310 | DHFRP1; RRM1; | Positive |
| kinase ARCHS4 | | | NASP; NCAPG; | |
| coexpression | | | POLR3G; MRPL47; | |
| | | | CENPN; SRSF7 | |
| CDK2 human | 0.00022 | 0.01310 | RRM1; MYBBP1A; | Positive |
| kinase ARCHS4 | | | FUS; NCAPG; | |
| coexpression | | | CABLES1; RRP1B; | |
| | | | GAPDH; IPO4 | |
| CDK4 human | 0.00022 | 0.01310 | CCND1; PUF60; | Positive |
| kinase ARCHS4 | | | MYBBP1A; RRP1B; | |
| coexpression | | | GAPDH; IPO4; | |
| | | | PFKP; MRPL11 | |
| CDC7 human | 0.00022 | 0.01310 | RRM1; NASP; | Positive |
| kinase ARCHS4 | | | TCP1; NCAPG; | |
| coexpression | | | POLR3G; CENPN; | |
| | | | RRP1B; SRSF7 | |
| BCKDK human | 0.00022 | 0.01310 | SYNGR2; MAPKAPK3; | Positive |
| kinase ARCHS4 | | | PUF60; HM13; | |
| coexpression | | | HSF1; JTB; | |
| | | | MRPL23; GAPDH | |
| Myc Targets V1 | 0.00076 | 0.02515 | RRM1; TCP1; | Positive |
| | | | PRPF31; EIF2S2; | |
| | | | MRPL23; SRSF7 | |
| Gene Expression | 0.00042 | 0.02967 | FUS; EIF2S2; | Positive |
| Homo sapiens | | | ELAVL1; NOB1; | |
| R-HSA-74160 | | | SRP72; MYBBP1A; | |
| | | | RRAGB; KAT6A; | |
| | | | ZMAT5; NABP2; | |
| | | | RPL24; TMEM219; | |
| | | | POLR3G; PRPF31; | |
| | | | RPS11; POLR2I; | |
| | | | SRSF7; TRIM33; | |
| | | | RPS12; PAIP1 | |
| Ribosome | 0.00022 | 0.03354 | RPL24; RPS11; | Positive |
| | | | MRPL23; MRPL32; | |
| | | | RPS12; MRPL11 | |
| Processing of | 0.00063 | 0.03841 | FUS; ZMAT5; | Positive |
| Capped Intron- | | | PRPF31; POLR2I; | |
| Containing Pre- | | | SRSF7; ELAVL1 | |
| mRNA Homo sapiens | | | | |
| R-HSA-72203 | | | | |
| CDK7 human | 0.00120 | 0.03899 | GLRX3; TCP1; | Positive |
| kinase ARCHS4 | | | POLR3G; MRPL47; | |
| coexpression | | | CENPN; MRPL32; | |
| | | | PAIP1 | |
| BUB1 human | 0.00120 | 0.03899 | RRM1; NASP; | Positive |
| kinase ARCHS4 | | | RAD21; TCP1; | |
| coexpression | | | NCAPG; CENPN; | |
| | | | RRP1B | |
| BUB1B human | 0.00120 | 0.03899 | RRM1; NASP; | Positive |
| kinase ARCHS4 | | | RAD21; TCP1; | |
| coexpression | | | NCAPG; CENPN; | |
| | | | RRP1B | |
| TTK human | 0.00120 | 0.03899 | RRM1; NASP; | Positive |
| kinase ARCHS4 | | | RAD21; TCP1; | |
| coexpression | | | NCAPG; POLR3G; | |
| | | | CENPN | |
| ADCK2 human | 0.00120 | 0.03899 | SYNGR2; MYBBP1A; | Positive |
| kinase ARCHS4 | | | TSPAN17; MGAT4B; | |
| coexpression | | | EDF1; PNPLA6; | |
| | | | MRPL23 | |
| mRNA Splicing - | 0.00080 | 0.04271 | FUS; PRPF31; | Positive |
| Major Pathway Homo | | | POLR2I; SRSF7; | |
| sapiens R-HSA-72163 | | | ELAVL1 | |

Further, a reactome pathway analysis was performed. Significantly up-regulated genes in cell subset 2 (high functional heterogeneity) were observed to be significantly involved in pathways responsible for cellular metabolism, showing correlation with higher functional heterogeneity from protein expression (FIG. 37A and FIG. 37B).

A combined heat map displaying both the proteomic and transcriptomic data obtained from the single cells was generated (FIG. 38). The clustering highlights the distinct makeup and behavior of these two cell subsets.

Proteomic and mRNA (transcriptomic) data was simultaneously obtained from 55 individual melanoma cells using the devices and methods described herein. Two distinct populations of cells were identified, one with low functional heterogeneity (subset 1), and another with high functional heterogeneity (simultaneous expression of multiple proteins) (subset 2). These two subpopulations each had a unique gene expression profile, indicating the possible genes that are regulating the heterogeneous protein expression of the cells. This type of combined data is capable of providing unprecedented insight into the link between functional heterogeneity and genetic make-up of individual cells. The insight this provides into the function of single cells will enable the manipulation of cells at the transcriptional and translational levels to alter cellular function and provide new therapeutic strategies. Biologically, this has implications for the understanding of directed and proteomically driven tumor biology. Leveraging these methods will enable a better understanding of what gene pathways regulate certain highly heterogeneous tumor cells, and the potential to start adjusting it at the transcription translation stage.

While various inventive embodiments have been described and illustrated herein, those of ordinary skill in the art will readily envision a variety of other means, structures, configurations, components, and the like, for performing a function, a step, and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, compounds, steps, and configurations described herein are meant to be merely an example and that the actual parameters, dimensions, materials, compounds steps, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure, and considered embodiments.

Embodiments disclosed herein may also be combined with one or more features, as well as complete systems, devices, and/or methods, known in the art, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as noted, various inventive concepts may be embodied as one or more methods, of which one or more examples have been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A multiplex assay chip device configured for multiplexed analysis of biological material, the device comprising:
a plurality of capture beads (CB) each bead including a capture moiety; and
a substrate having a plurality of chambers each including
an open end arranged on a first side of the substrate, and a length, a width, and a depth (“dimensions”),
a pocket P1 having a pocket diameter greater than the width of the chamber, and
at least one CB arranged within the pocket of each chamber,
wherein each CB includes a CB diameter smaller than the pocket diameter and larger than the width of the chamber; and
a surface configured to couple with the first side of the substrate to cover each chamber.

2. A multiplex assay chip device configured for multiplexed analysis of biological material, the device comprising:
a plurality of capture beads (CB) each bead including a capture moiety;
a substrate having a plurality of chambers each including
an open end arranged on a first side of the substrate, and a length, a width, and a depth (“dimensions”),
a plurality of pockets comprising at least a first pocket P1 and a second pocket P2, each pocket of the plurality of pockets having a pocket diameter greater than the width of the chamber, and
at least one CB arranged within each pocket of each chamber, wherein each CB includes a CB diameter smaller than the pocket diameter and larger than the width of the chamber; and
a surface configured to couple with the first side of the substrate to cover each chamber.

3. The multiplex assay chip of claim 1, wherein the surface comprises a plurality of substantially parallel lines of capture antibodies, each line of capture antibodies comprises a different antibody, relative to antibodies of adjacent lines of capture antibodies, configured to bind to a different target molecule, and at least one portion of each of the plurality of lines are arranged as to be exposed to each chamber.

4. The device of claim 2, wherein P1 and P2 have the same pocket diameter.

5. The device of claim 2, wherein P1 and P2 have different pocket diameters.

6.-8. (canceled)

9. The device of claim 1, wherein P1 has a pocket diameter of between 5 μm and 50 μm larger than the chamber width.

10. The device of claim 1, wherein P1 has a pocket diameter of between 10 μm and 100 μm.

11. The device of claim 2, wherein P2 has a pocket diameter of between 5 μm and 50 μm larger than the P1 pocket diameter.

12. The device of claim 2, wherein P2 has a pocket diameter of between 15 μm and 150 μm.

13. The device of claim 1, wherein the diameter of the CB of P1, CB1, is between 0 μm and 50 μm smaller than the diameter of P1.

14. The device of claim 2, wherein the diameter of the CB of P2, CB2, is between 0 μm and 50 μm smaller than the diameter of P2.

15. The device of claim 2, wherein the diameter of the CB in P2, CB2 is larger than the diameter of P1.

16. The device of claim 2, wherein the CB of P2, CB2, does not fit within P1.

17. The device of claim 2, wherein P1 and P2 are center-aligned with respect to the width of the chamber.

18. The device of claim 2, wherein P1 and P2 are not center-aligned with respect to the width of the chamber.

19. The device of claim 2, wherein P1 and P2 are non-overlapping.

20.-21. (canceled)

22. The device of claim 1, wherein the capture moiety is configured to capture nucleic acid sequences, peptides, proteins, metabolites, or organic molecules.

23. The device of claim 22, wherein the capture moiety is configured to capture nucleic acid sequences.

24. The device of claim 22, wherein the captured nucleic acid sequence is DNA, RNA, or a combination thereof.

25. The device of claim 24, wherein the RNA is an mRNA.

26. The device of claim 22, wherein the nucleic acid capturing CB is an oligonucleotide capture bead comprising a nucleic acid capture sequence tethered to a bead.

27. (canceled)

28. The device of claim 26, wherein the nucleic acid capturing CB nucleic acid capture sequence comprises an individually unique cell barcode sequence, a PCR handle, a unique molecular identifier (UMI), a barcode handle sequence, and a capture sequence.

29. The device of claim 26, wherein the capture sequence comprises a polyT sequence for mRNA polyA capture.

30. The device of claim 26, wherein the capture sequence comprises a gene-specific or sequence-specific capture sequence.

31. The device of claim 22, wherein each nucleic acid sequence of the nucleic acid capturing CB comprises a unique UMI.

32. The device of claim 28, wherein the cell barcode sequence of the CB is unique to each CB of the plurality of CBs.

33. The device of claim 28, wherein the cell barcode sequence of the CB is unique to each chamber of the plurality of chambers.

34.-35. (canceled)

36. The device of claim 1, wherein the bead is porous.

37.-42. (canceled)

43. The device claim 1, wherein the biological material is a biological sample, a metabolite, a protein, a polypeptide, or a cell.

44. The device of claim 1, wherein the cell is a single cell.

45.-86. (canceled)

87. A multiplexed method for the simultaneous identification of:
(a) at least one nucleic acid sequence, and (b) at least one protein from a single subject cell comprising:
(I) providing a device comprising:
a substrate having a plurality of chambers each comprising at least one capture bead (CB), wherein a first CB, CB1, is configured to capture a target nucleic acid; and
a surface configured to couple with a first side of the substrate to cover each chamber wherein: the surface comprises a plurality of substantially parallel lines of capture antibodies, each line of capture antibodies comprising a different antibody, configured to bind to a different target molecule, and at least one portion of each of the plurality of substantially parallel lines are arranged as to be exposed to each chamber;
(II) introducing the subject cell to a chamber of the plurality of chambers;
(III) maintaining the subject cell in the chamber under conditions sufficient to cause cell lysis to produce a cell lysate;
(IV) identifying at least one first nucleic acid sequence, comprising:
(a) providing conditions sufficient for the target nucleic acid to contact the CB1 to form a CB1-target nucleic acid complex,
(b) determining the sequence of the complexed target nucleic acid to determine the sequence of the at least one expressed target nucleic acid sequence
(V) detecting the at least one protein in the subject cell comprising the steps of:
(a) incubating the cell lysate in the chamber under conditions sufficient to allow at least one antibody and at least one protein to form an antibody:protein complex; and
(b) imaging the surface comprising the at least one antibody:protein complex, thereby identifying the one or more protein of the subject cell.

88.-134. (canceled)
